(19) United States
(12) Patent Application Publication
(10) Pub. No.: US 2025/0177530 A1
(43) Pub. Date: Jun. 5, 2025

(54) MOLECULES THAT BIND TO ENPP1 POLYPEPTIDES

(71) Applicants: UNIVERSITY OF PITTSBURGH - OF THE COMMONWEALTH SYSTEM OF HIGHER EDUCATION, Pittsburgh, PA (US); Provincial Health Services Authority

(72) Inventors: Dimiter Stanchev Dimitrov, Pittsburgh, PA (US); Dusan Baek, Pittsburgh, PA (US); Xiaojie Chu, Pittsburgh, PA (US); Poul H. Sorensen, Vancouver (CA)

(21) Appl. No.: 18/875,888

(22) PCT Filed: Jun. 23, 2023
(86) PCT No.: PCT/US2023/026047

§ 371 Date: Dec. 17, 2024

Related U.S. Application Data
(60) Provisional application No. 63/367,021, filed on Jun. 24, 2022.

(51) Int. Cl. A61K 40/42; A61K 40/11; A61K 40/31; C07K 16/28; C07K 16/40

(52) U.S. Cl. CPC A61K 40/4224; A61K 40/11; A61K 40/31; A61K 40/4252; C07K 16/2896; C07K 16/40; C07K 2317/622; C07K 2317/92

(57) ABSTRACT

This document provides methods and materials involved in binding a binder (e.g., an antibody, antigen binding fragment, antibody domain, CAR, cell engager, and/or ADC) to an ENPP1 polypeptide. For example, binders (e.g., antibodies, antigen binding fragments, antibody domains, CARs, cell engagers, and/or ADCs) that bind to an ENPP1 polypeptide and methods and materials for using one or more such binding molecules to treat a mammal (e.g., a human) having cancer (e.g., one or more sarcomas) are provided.

### CROSS-REFERENCE TO RELATED APPLICATIONS

This application claims the benefit of U.S. Patent Application Ser. No. 63/367,021, filed on Jun. 24, 2022. The disclosure of the prior application is considered part of, and is incorporated by reference in, the disclosure of this application.

### STATEMENT REGARDING FEDERAL FUNDING

This invention was made with government support under CA232561 awarded by the National Institutes of Health. The government has certain rights in the invention.

### SEQUENCE LISTING

This application contains a Sequence Listing that has been submitted electronically as an XML file named “48881-0041WO1.xml.” The XML file, created on May 25, 2023, is 277000 bytes in size. The material in the XML file is hereby incorporated by reference in its entirety.

### TECHNICAL FIELD

This document relates to methods and materials involved in binding a molecule (e.g., an antibody, a fragment of an antibody, an antibody domain, a chimeric antigen receptor (CAR), a cell engager, or an antibody-drug conjugate (ADC)) to an ectonucleotide pyrophosphatase/phosphodiesterase family member 1 (ENPP1) polypeptide. For example, this document provides binders (e.g., antibodies, antigen binding fragments, antibody domains, CARs, cell engagers, or ADCs) that bind to an ENPP1 polypeptide and methods and materials for using such binders to treat cancer (e.g., one or more sarcomas). This document also provides cells (e.g., host cells) designed to express one or more binders (e.g., antibodies, antigen binding fragments, antibody domains, CARs, or cell engagers) having the ability to bind to an ENPP1 polypeptide and methods and materials for using such cells to treat cancer (e.g., one or more sarcomas).

### BACKGROUND INFORMATION

The ENPP1 gene is a member of the ENPP family, and the encoded ENPP1 polypeptide is a type II transmembrane glycoprotein comprising two identical disulfide-bonded subunits. The ENPP1 polypeptide can cleave a variety of substrates, including adenosine triphosphate (ATP), which is cleaved into adenosine monophosphate (AMP) and diphosphate, and nicotinamide adenine dinucleotide (NAD⁺), which can be hydrolyzed to produce AMP. In a tumor microenvironment, AMP generated by an ENNP1 polypeptide can lead to production of adenosine, which can suppress the anti-cancer function of the immune system.

### SUMMARY

This document provides methods and materials involved in binding a molecule (e.g., an antibody, an antigen binding fragment, an antibody domain, a CAR, a cell engager, or an ADC) to an ENPP1 polypeptide. For example, this document provides binders (e.g., antibodies, antigen binding fragments, antibody domains, CARs, cell engagers, or ADCs) that bind to an ENPP1 polypeptide and methods and materials for using one or more such binders to treat a mammal (e.g., a human) having cancer (e.g., one or more sarcomas).

This document also provides cells (e.g., host cells) designed to express one or more binders (e.g., antibodies, antigen binding fragments, antibody domains, CARs, or cell engagers) having the ability to bind to an ENPP1 polypeptide and methods and materials for using such cells to treat cancer (e.g., one or more sarcomas).

As described herein, binders (e.g., one or more antibodies, one or more antigen binding fragments, one or more antibody domains, one or more CARs, one or more cell engagers, and/or one or more ADCs) can be designed to have the ability to bind to an ENPP1 polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human ENPP1 polypeptide as set forth in SEQ ID NO:150 or SEQ ID NO:151 (see, e.g., Example 2).

In some cases, two sets of three CDRs of an antigen binding fragment provided herein (e.g., SEQ ID NOs: 1-3 and 9-11 or SEQ ID NOs: 17-19 and 25-27) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target ENPP1+ cells (e.g., ENPP1⁺ tumor cells), can be engineered into an antibody structure that includes an Fc region to create antibodies having the ability to target ENPP1⁺ cells (e.g., ENPP1⁺ tumor cells) and induce antibody-dependent cell-mediated cytotoxicity (ADCC) against the target ENPP1⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target ENPP1⁺ cells (e.g., ENPP1⁺ tumor cells) and induce one or more immune responses (e.g., T cell immune responses and/or ADCC using a cell engager in the absence of an Fc-containing antibody) against the target ENPP1⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., ENPP1⁺ cancer cells).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer (e.g., one or more sarcomas). For example, a mammal (e.g., a human) having cancer (e.g., a ENPP1⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to an ENPP1 polypeptide. For example, cells such as T cells (e.g., CTLs), stem cells (e.g., induced pluripotent stem cells), or NK cells can be engineered to express one or more CARs having the ability to bind to an ENPP1polypeptide. Such cells (e.g., ENPP1-specific CAR⁺ T cells or NK cells) can be used to treat cancer (e.g., one or more sarcomas).

In some cases, a binder (e.g., an antibody, antigen binding fragment, and/or antibody domain) provided herein can be used to detect the presence or absence of an ENPP1 polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains ENPP1⁺ cells (e.g., ENPP1″ cancer cells). Having the ability to detect the presence or absence of an ENPP1 polypeptide (e.g., ENPP1⁺ cancer cells) can allow clinicians, health professionals, and patients to make better decisions about possible treatment options. For example, detection of ENPP1⁺ cancer cells within a mammal can allow clinicians, health professionals, and patients to select an appropriate anti-cancer treatment that targets the ENPP1⁺ cancer cells. Such treatments that targets the ENPP1⁺ cancer cells can include administration of an inhibitor of ENPP1 polypeptide activity such as RBS2418 and/or one or more of the binders described herein having the ability to bind to a ENPP1 polypeptide and/or administration of one or more cells (e.g., ENPP1-specific CAR⁺ T cells or NK cells) designed to express a binder described herein.

In general, one aspect of this document features antibodies including: (i) a heavy chain variable domain or region comprising the amino acid sequences set forth in SEQ ID NO: 1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO: 19 with one, two, or three amino acid additions, deletions, or substitutions), and a light chain variable domain or region comprising the amino acid sequences set forth in SEQ ID NO: 25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can have the ability to bind to SEQ ID NO: 150 or SEQ ID NO: 151. The antibody can include the heavy chain variable domain or region of (i). The heavy chain variable domain or region can include an amino acid sequence having at least 90 percent identity to the amino acid sequence set forth in SEQ ID NO:8. The antibody can include the light chain variable domain or region of (i). The light chain variable domain or region can include an amino acid sequence having at least 90 percent identity to the amino acid sequence set forth in SEQ ID NO:16. The antibody can include the heavy chain variable domain or region of (ii). The heavy chain variable domain or region can include an amino acid sequence having at least 90 percent identity to the amino acid sequence set forth in SEQ ID NO:24. The antibody can include the light chain variable domain or region of (ii). The light chain variable domain or region can include an amino acid sequence having at least 90 percent identity to the amino acid sequence set forth in SEQ ID NO: 32. The antibody can be a monoclonal antibody. The antibody can be an scFv antibody.

In another aspect, this document features antigen binding fragments including: (i) a heavy chain variable domain or region comprising the amino acid sequences set forth in SEQ ID NO: 1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO: 19 with one, two, or three amino acid additions, deletions, or substitutions), and a light chain variable domain or region comprising the amino acid sequences set forth in SEQ ID NO: 25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can have the ability to bind to SEQ ID NO: 150 or SEQ ID NO:151. The antigen binding fragment can include the heavy chain variable domain or region of (i). The heavy chain variable domain or region can include an amino acid sequence having at least 90 percent identity to the amino acid sequence set forth in SEQ ID NO:8. The antigen binding fragment can include the light chain variable domain or region of (i). The light chain variable domain or region can include an amino acid sequence having at least 90 percent identity to the amino acid sequence set forth in SEQ ID NO: 16. The antigen binding fragment can include the heavy chain variable domain or region of (ii). The heavy chain variable domain or region can include an amino acid sequence having at least 90 percent identity to the amino acid sequence set forth in SEQ ID NO:24. The antigen binding fragment can include the light chain variable domain or region of (ii). The light chain variable domain or region can include an amino acid sequence having at least 90 percent identity to the amino acid sequence set forth in SEQ ID NO:32. The antigen binding fragment can be monoclonal. The antigen binding fragment can be an Fab.

In another aspect, this document features chimeric antigen receptors including an antigen binding domain, a hinge, a transmembrane domain, and one or more signaling domains, where the antigen binding domain includes an antibody or an antigen-binding fragment including: (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO: 18 with one, two, or three amino acid additions, deletions, or substitutions), and SEQ ID NO: 19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO: 27 with one, two, or three amino acid additions, deletions, or substitutions). The antigen binding domain can include a scFv having the ability to bind to an ENPP1 polypeptide. The hinge can be a hinge set forth in any one of SEQ ID NOs: 105-112. The transmembrane domain can be a transmembrane domain set forth in any one of SEQ ID NOs: 113-120. The chimeric antigen receptor can include one or more signaling domains set forth in any one of SEQ ID NOs: 121-129.

In another aspect, this document features cells comprising a chimeric antigen receptor including an antigen binding domain, a hinge, a transmembrane domain, and one or more signaling domains, where the antigen binding domain includes an antibody or an antigen-binding fragment including: (i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO: 3 with one, two, or three amino acid additions, deletions, or substitutions), and a light chain variable domain or region comprising the amino acid sequences set forth in SEQ ID NO: 9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO: 17 with one, two, or three amino acid additions, deletions, or substitutions), SEQ ID NO: 18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The cell can be a T cell, a stem cell, or an NK cell.

In another aspect, this document features cell engagers including a first antigen binding domain, a linker, and a second antigen binding domain, wherein said first antigen binding domain comprises an antibody or an antigen-binding fragment including: (i) a heavy chain variable domain or region comprising the amino acid sequences set forth in SEQ ID NO: 1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO: 19 with one, two, or three amino acid additions, deletions, or substitutions), and a light chain variable domain or region comprising the amino acid sequences set forth in SEQ ID NO: 25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The first antigen binding domain can include a scFv having the ability to bind to an ENPP1 polypeptide. The first antigen binding domain can be an IgG having the ability to bind to an ENPP1 polypeptide. The linker can be a linker set forth in any one of SEQ ID NOs: 76-102 or SEQ ID NOs: 105-112. The second antigen binding domain can bind to a polypeptide expressed on the surface of T cells. The polypeptide expressed on the surface of T cells can be a CD3 polypeptide. The second antigen binding domain can be an antigen binding domain set forth in Example 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in Example 20. The cell engager can include a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in Example 20.

In another aspect, this document features nucleic acid including a nucleic acid sequence encoding at least part of an antibody or an antigen-binding fragment described above. The nucleic acid sequence can encode: (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO: 2 with one, two, or three amino acid additions, deletions, or substitutions), and SEQ ID NO: 3 (or SEQ ID NO:3 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO: 18 with one, two, or three amino acid additions, deletions, or substitutions), and SEQ ID NO: 19 (or SEQ ID NO: 19 with one, two, or three amino acid additions, deletions, or substitutions). The nucleic acid sequence can encode: (i) a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO: 11 with one, two, or three amino acid additions, deletions, or substitutions); or (ii) a light chain variable domain or region comprising the amino acid sequences set forth in SEQ ID NO: 25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The nucleic acid can be a viral vector. The nucleic acid can be a phagemid.

In another aspect, this document features nucleic acid including a nucleic acid sequence encoding a chimeric antigen receptor described above or a cell engager described above. The nucleic acid can be a viral vector. The nucleic acid can be a phagemid.

In another aspect, this document features host cells including a nucleic acid described above. The host cell can be a T cell, stem cell, or NK cell.

In another aspect, this document features host cells that expresses a chimeric antigen receptor described above or a cell engager described above. The host cell can be a T cell, stem cell, or NK cell.

In another aspect, this document features ADC including an antigen binding domain covalently linked to a drug, where the antigen binding domain includings an antibody or an antigen binding fragment described above. The antigen binding domain can include a scFv having the ability to bind to an ENPP1 polypeptide. The antigen binding domain can be an IgG having the ability to bind to an ENPP1 polypeptide. The drug can be selected from the group consisting of auristatins, mertansine, and pyrrolobenzodiazepine (PBD) dimers.

In another aspect, this document features compositions including an antibody or an antigen binding fragment described above. The composition can include antibody described above. The composition can include an antigen binding fragment described above. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features compositions including a cell engager described above. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features compositions including a cell described above. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features compositions including an ADC described above. The composition can include a checkpoint inhibitor. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab.

In another aspect, this document features methods for treating a mammal having cancer. The methods can include, or consist essentially of, administering, to a mammal having cancer, a composition described above. The mammal can be a human. The cancer can be an ENPP1⁺ cancer. The ENPP1⁺ cancer can be an ENPP1⁺ sarcoma, an ENPP1⁺ lung cancer, an ENPP1⁺ breast cancer, or an ENPP1⁺ ovarian cancer. The number of cancer cells within the mammal can be reduced following the administering step.

In another aspect, this document features methods for treating a mammal having cancer. The methods can include, or consist essentially of, (a) administering, to a mammal having cancer, a composition described above, and (b) administering, to the mammal, a composition including a checkpoint inhibitor. The mammal can be a human. The cancer can be an ENPP1⁺ cancer. The ENPP1⁺ cancer can be an ENPP1⁺ sarcoma, an ENPP1⁺ lung cancer, an ENPP1⁺ breast cancer, or an ENPP1⁺ ovarian cancer. The checkpoint inhibitor can be cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, or ipilimumab. The number of cancer cells within the mammal can be reduced following the administering steps (a) and (b).

In another aspect, this document features methods for method for binding a binding molecule to an ENPP1 polypeptide. The methods can include, or consist essentially of, contacting an ENPP1 polypeptide with an antibody or an antigen binding fragment described above. The contacting can be performed in vitro. The contacting can be performed in vivo. The contacting can be performed within a mammal by administering the antibody or the antigen binding fragment to the mammal. The mammal can be a human.

In another aspect, this document features methods for binding a binding molecule to an ENPP1 polypeptide. The methods can include, or consist essentially of, contacting an ENPP1 polypeptide with a chimeric antigen receptor described above, a cell engager described above, or an ADC described above. The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1F. Specificity of Fab/IgG1 with recombinant human ENPP1 and human membrane proteins. FIG. 1A, Fab 17, Fab 3G12, IgG1 17, and IgG1 3G12 binding to recombinant human ENPP1 measured by ELISA. Experiments were performed in duplicate. FIG. 1B, Aggregation evaluation of IgG1 17 and IgG1 3G12 measured by SEC. FIG. 1C, Binding of Fab 17 and 3G12 to BSA measured by ELISA. FIG. 1D, Competitive ELISA of IgG1 17 with serial diluted IgG1 3G12 for binding to ENPP1. FIG. 1E, Detection of ENPP1 cell surface expression level on 293T, 293T-ENPP1, HepG2, PC3, and CHLA10 cell lines. FIG. 1F, Cell surface binding of IgG1 17 and 3G12 (100 nM) on 293T, 293T-ENPP1, and HepG2 cells.

FIGS. 2A-2D. Cytotoxicity of IgG1-MMAE to ENPP1-expressing cells. FIG. 2A, Representative pictures of ADC cytotoxicity, MMAE drug alone or IgG1 alone at 50 nM on 293T-ENPP1 cell, 293T cell, and HepG2 cell separately. Cells were plated into a 96-well plate and treated with MMAE-IgG1 or IgG1 and MMAE alone at different concentrations for 4 days. FIGS. 2B-2D, % Relative cell viability of 293T-ENPP1 cells (FIG. 2B), 293T cells (FIG. 2C), and HepG2 cells (FIG. 2D) after treatment with MMAE, IgG1 17/3G12 and MMAE-IgG1 17/3G12 separately. Experiments were performed in duplicate. Values were reported as the mean of percent relative cell viability±SD. Experiment was repeated three times. Significance was tested by using two-way ANOVA, followed by Tukey's multiple comparisons test. ****, p<0.0001, *, p<0.05.

FIGS. 3A-3F. In vitro cytotoxic of T cells to ENPP1-expressing cells induced by anti-ENPP1 BiTE. FIG. 3A, Aggregation evaluation of BiTE 3G12 and IgG1 3G12 measured by SEC. FIGS. 3B-3C, BiTE 17 and 3G12 binding to ENPP1 (FIG. 3B) and CD3 (FIG. 3C) measured by ELISA. FIGS. 3D-F, Percent relative lysis of T cells to 293T-ENPP1 cell (FIG. 3D), 293T cell (FIG. 3E), and HepG2 cancer cell (FIG. 3F) mediated by BiTE 17 and 3G12 separately. T cells and target cells were added to the 96-well plate at E:T ratio of 10:1 and simultaneously treated with serially diluted BiTE antibodies for 24 hours. The luminescence signal was detected and used to calculate the percent relative lysis. Experiments were performed in duplicate. Values were reported as the mean of percent relative lysis±SD. Experiment was repeated three times. Significance was tested by using two-way ANOVA, followed by Tukey's multiple comparisons test. ****, p<0.0001.

FIGS. 4A-4G. In vitro cytotoxicity of CAR T to ENPP1-expressing cells. FIG. 4A, Cell surface binding of scFv format antibody 17 and 3G12 on 293T, 293T-ENPP1, and HepG2 cells. FIG. 4B, scFv 17 and 3G12 binding to recombinant human ENPP1 measured by ELISA. Experiments were performed in duplicate. FIG. 4C, CAR expression level on CD3⁺CD4⁺ and CD3⁺CD8⁺ T population in anti-ENPP1 CAR T cells. FIGS. 4D-4F, Percent relative lysis of anti-ENPP1 CAR T 17 and 3G12 against 293T, 293T-ENPP1 and HepG2 separately. FIG. 4G, Granzyme B and IFN-γ secretion in cell supernatant from Control T or CAR T at 24-hour incubation with 293T-ENPP1 target cell at different ratios were detected by ELISA. Values were reported as the mean±SD. Experiment was repeated three times. Significance was tested by using two-way ANOVA, followed by Tukey's multiple comparisons test. ****, p<0.0001. ***, p<0.001, **, p<0.01, *, p<0.05.

FIGS. 5A-5D. Affinity characterization of different format anti-hENPP1 antibodies by BLItz. FIGS. 5A-5B, Kinetics of Fab 17 (FIG. 5A) and Fab 3G12 (FIG. 5B) binding to recombinant ENPP1 measured by BLItz. FIGS. 5C-5D, Kinetics of IgG1 17 (FIG. 5C) and IgG1 3G12 (FIG. 5D) binding to recombinant ENPP1 measured by BLItz.

FIG. 6. In vitro ADCC activity characterization. ADCC activity of anti-ENPP1 IgG1 17, IgG1 3G12, and isotype IgG1 against 293-ENPP1 cell. Values were reported as the mean±SD. Significance was tested by using two-way ANOVA, followed by Tukey's multiple comparisons test. ***, p<0.001.

FIGS. 7A-7B. Internalization of IgG1 by ENPP1 positive and negative cell lines. FIG. 7A, The internalization efficiency of IgG1 17 on 293T cells and 293T-ENPP1 cells. FIG. 7B, The internalization efficiency of IgG1 3G12 on 293T cells and 293T-ENPP1 cells. The IgG1 17 and 3G12 labeled with pHrodo™ Deep Red were incubated with 293T and 293T-ENPP1 cells separately at 37° C. for 2 hours.

FIG. 8 is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a scFv provided herein (e.g., a scFv designed to include two sets of three CDRs such as CDR1, CDR2, and CDR3 of a heavy chain and CDR1, CDR2, and CDR3 of a light chain (in either order) of an antigen binding fragment provided herein, for example, SEQ ID NOs: 1-3 and 9-11 or SEQ ID NOs: 17-19 and 25-27), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3 intracellular signaling domain sequence).

FIG. 9 is a schematic of an exemplary BiTE designed using CDR1, CDR2, and CDR3 of a heavy chain provided herein and CDR1, CDR2, and CDR3 of a light chain provided herein in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (G₄S)₃ (SEQ ID NO:78) linker).

### DETAILED DESCRIPTION

This document provides binders (e.g., antibodies, antigen binding fragments, antibody domains, CARs, cell engagers, and ADCs) that bind (e.g., specifically bind) to an ENPP1 polypeptide (e.g., a human ENPP1 polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:150 or SEQ ID NO: 151 (see, e.g., Example 2). In some cases, a binder (e.g., an antibody, an antigen binding fragment, an antibody domain, a CAR, a cell engager, or an ADC) provided herein can have the ability to bind to an ENPP1 polypeptide and can lack the ability to bind to other members of the ENPP family (e.g., an ENPP2 polypeptide, an ENPP3 polypeptide, an ENPP4 polypeptide, an ENPP5 polypeptide, an ENPP6 polypeptide, and an ENPP7 polypeptide). For example, a binder (e.g., an antibody, an antigen binding fragment, an antibody domain, a CAR, a cell engager, or an ADC) provided herein can have the ability to bind to a human ENPP1 polypeptide and can lack the ability to bind to one or more other human ENPP polypeptides (e.g., a human ENPP2 polypeptide, a human ENPP3 polypeptide, a human ENPP4 polypeptide, a human ENPP5 polypeptide, a human ENPP6 polypeptide, and/or a human ENPP7 polypeptide).

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Färber-Schwarz, Methods Mol. Biol., 907:713-27 (2012); and Brinkmann and Kontermann, MAbs., 9 (2): 182-212 (2017)). The two variable regions can be connected by a polypeptide linker (e.g., a polypeptide linker having five to ten residues in length or a polypeptide linker as set forth in Example 11). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in Example 11). See, also, Chen et al., Adv. Drug Deliv. Rev., 65 (10): 1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in Example 7, Example 8, Example 9, and Example 10.

An antibody provided herein can include the CDRs as described herein (e.g., as described in Table 13) and can be configured to be a human antibody, a humanized antibody, or a chimeric antibody. In some cases, an antibody provided herein can include the CDRs as described herein (e.g., as described in Table 13) and can be a monoclonal antibody. In some cases, an antibody provided herein can include the CDRs as described herein (e.g., as described in Table 13) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 13) and can be configured to be a human antigen binding fragment, a humanized antigen binding fragment, or a chimeric antigen binding fragment. In some cases, an antigen binding fragment provided herein can include the CDRs as described herein (e.g., as described in Table 13) and can be a monoclonal antigen binding fragment. In some cases, an antigen binding fragment provided herein can include the CDRs as described herein (e.g., as described in Table 13) and can be configured as an Fab antibody. In some cases, a Fab antibody can include a partial hinge sequence (e.g., SEQ ID NO: 152) for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 13) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 13) and can be a monoclonal antibody domain. In some cases, an antibody domain provided herein can include the CDRs as described herein (e.g., as described in Table 13) and can be engineered as a single VH domain or a single VL domain.

An anti-ENPP1 antibody, anti-ENPP1 antigen binding fragment, or anti-ENPP1 antibody domain provided herein can be of the IgA-, IgD-, IgE-, IgG-, or IgM-type, including IgG- or IgM-types such as, without limitation, IgG1-, IgG2-, IgG3-, IgG4-, IgM1-, and IgM2-types. In some cases, an antibody provided herein (e.g., an anti-ENPP1 antibody) can be a scFv antibody. In some cases, an antigen binding fragment provided herein (e.g., an anti-ENPP1 antibody fragment) can be an Fab. In some cases, an antibody provided herein (e.g., an anti-ENPP1 antibody) can be a fully intact antibody having the structure set forth in Example 6. In some cases, an antibody domain provided herein (e.g., an anti-ENPP1 antibody domain) can be a VH domain.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to an ENPP1 polypeptide (e.g., a human ENPP1 polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to an ENPP1 polypeptide (e.g., a human ENPP1 polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs: 1-3 and 9-11 or SEQ ID NOs: 17-19 and 25-27). In some cases, an antigen binding domain of a CAR targeting an ENPP1 polypeptide can be designed to include a VH domain described herein or a scFv antibody described herein.

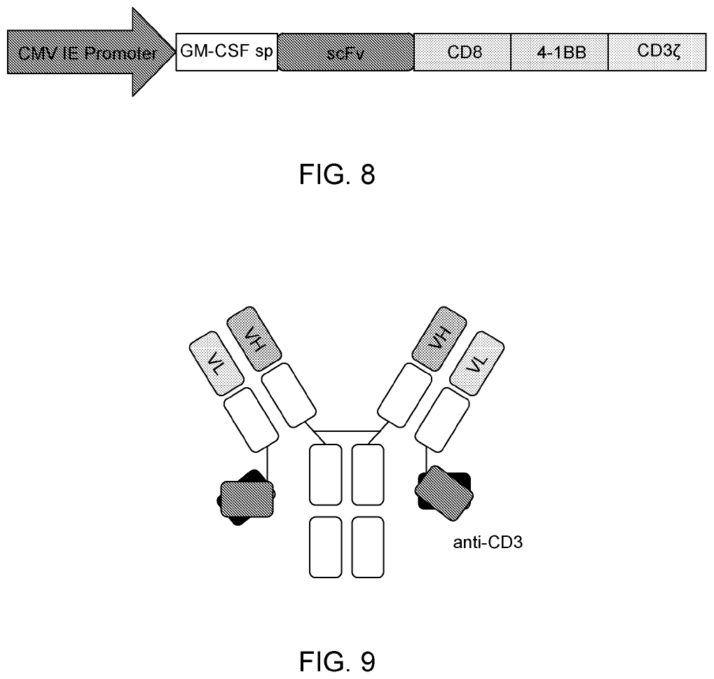

Examples of CAR structures that can be used to make a CAR provided herein include, without limitation, those set forth in Example 12 and FIG. 8.

In some cases, a CAR provided herein can be designed to include a signal peptide. Any appropriate signal peptide can be used to design a CAR described herein. Examples of signal peptide that can be used to make a CAR described herein include without limitation, human IGKV1-39-derived signal peptides, IGKV1-16 signal peptides, IGKV1-33 signal peptides, IGKV3-11 signal peptides, IGKV4-1 signal peptides, IGKV6-21 signal peptides, GM-CSF signal peptides, IL-2 signal peptides, and CD8a signal peptides. In some cases, a CAR provided herein can be designed to include a signal peptide that comprises, consists essentially of, or consists of one of the amino acid sequences set forth in Example 13. In some cases, a CAR provided herein can be designed to include a signal peptide that comprises, consists essentially of, or consists of one of the amino acid sequences set forth in Example 13 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in Example 13 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CH2 domain and a CH3 domain, Ig-derived hinges containing a CH2 domain and lacking a CH3 domain, Ig-derived hinges containing a CH3 domain and lacking a CH2 domain, Ig-derived hinges lacking a CH2 domain and lacking a CH3 domain, CD8α-derived hinges, CD28-derived hinges, and CD32-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as a hinge to make a CAR described herein. For example, any one of the linker sequences set forth in Example 11 can be used as a hinge of a CAR described herein.

In some cases, a CAR provided herein can be designed to include a hinge that comprises, consists essentially of, or consists of one of the amino acid sequences set forth in Example 11 or Example 14. In some cases, a CAR provided herein can be designed to include a hinge that comprises, consists essentially of, or consists of one of the amino acid sequences set forth in Example 11 or Example 14 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in Example 11 or Example 14 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. Examples of transmembrane domains that can be used to make a CAR described herein include, without limitation, CD33 transmembrane domains, CD4 transmembrane domains, CD8a transmembrane domains, CD28 transmembrane domains, 4-1BB transmembrane domains, and ICOS transmembrane domains. In some cases, a CAR provided herein can be designed to include a transmembrane domain that comprises, consists essentially of, or consists of one of the amino acid sequences set forth in Example 15. In some cases, a CAR provided herein can be designed to include a transmembrane domain that comprises, consists essentially of, or consists of one of the amino acid sequences set forth in Example 15 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in Example 15 with two or less, three or less, four or less, five or less, six or less, seven or less, eight or less, nine or less, or ten or less amino acid deletions, additions, substitutions, or combinations thereof.

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, DAP12 intracellular signaling domains, ICOS intracellular domains, MYD88-CD40 intracellular domains, and KIR2DS2 intracellular domains. In some cases, a CAR described herein can be designed to be a first generation CAR having a CD32 intracellular signaling domain. In some cases, a CAR described herein can be designed to be a second generation CAR having a CD28 intracellular signaling domain followed by a CD3G intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD32 intracellular signaling domain. In some cases, a CAR provided herein can be designed to include at least one intracellular signaling domain that comprises, consists essentially of, or consists of one of the amino acid sequences set forth in Example 16. In some cases, a CAR provided herein can be designed to include at least one intracellular signaling domain that comprises, consists essentially of, or consists of one of the amino acid sequences set forth in Example 16 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in Example 16 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting an ENPP1 polypeptide can be designed to include an scFv having a heavy chain variable domain comprising SEQ ID NO:1, SEQ ID NO:2, and SEQ ID NO:3, followed by a linker such as a linker set forth in Example 11, followed by a light chain variable domain comprising SEQ ID NO:9, SEQ ID NO:10, and SEQ ID NO:11, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD32 intracellular signaling domain). For example, a CAR targeting an ENPP1 polypeptide can be designed to include an scFv having a heavy chain variable domain comprising SEQ ID NO:1, SEQ ID NO:2, and SEQ ID NO:3, followed by SEQ ID NO:100, followed by a light chain variable domain comprising SEQ ID NO:9, SEQ ID NO:10, and SEQ ID NO:11, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

In some cases, a CAR targeting an ENPP1 polypeptide can be designed to include an scFv having a heavy chain variable domain comprising SEQ ID NO:8, followed by a linker such as a linker set forth in Example 11, followed by a light chain variable domain comprising SEQ ID NO: 16, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD3 (intracellular signaling domain). For example, a CAR targeting an ENPP1 polypeptide can be designed to include an scFv having a heavy chain variable domain comprising SEQ ID NO:8, followed by SEQ ID NO: 100, followed by a light chain variable domain comprising SEQ ID NO:16, followed by SEQ ID NO:102, followed by SEQ ID NO: 108, followed by SEQ ID NO: 119, followed by SEQ ID NO: 124, followed by SEQ ID NO: 123, followed by SEQ ID NO:97, followed by SEQ ID NO:121.

In some cases, a CAR targeting an ENPP1 polypeptide can be designed to include an scFv having a light chain variable domain comprising SEQ ID NO:9, SEQ ID NO: 10, and SEQ ID NO:11, followed by a linker such as a linker set forth in Example 11, followed by a heavy chain variable domain comprising SEQ ID NO:1, SEQ ID NO:2, and SEQ ID NO:3, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD32 intracellular signaling domain). For example, a CAR targeting an ENPP1 polypeptide can be designed to include an scFv having a light chain variable domain comprising SEQ ID NO:9, SEQ ID NO: 10, and SEQ ID NO: 11, followed by SEQ ID NO: 100, followed by a heavy chain variable domain comprising SEQ ID NO:1, SEQ ID NO: 2, and SEQ ID NO:3, followed by SEQ ID NO: 102, followed by SEQ ID NO: 108, followed by SEQ ID NO:119, followed by SEQ ID NO:124, followed by SEQ ID NO: 123, followed by SEQ ID NO:97, followed by SEQ ID NO: 121 (see, e.g., Example 17).

In some cases, a CAR targeting an ENPP1 polypeptide can be designed to include an scFv having a light chain variable domain comprising SEQ ID NO:16, followed by a linker such as a linker set forth in Example 11, followed by a heavy chain variable domain comprising SEQ ID NO:8, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD35 intracellular signaling domain). For example, a CAR targeting an ENPP1 polypeptide can be designed to include an scFv having a light chain variable domain comprising SEQ ID NO:16, followed by SEQ ID NO:100, followed by a heavy chain variable domain comprising SEQ ID NO:8, followed by SEQ ID NO:102, followed by SEQ ID NO: 108, followed by SEQ ID NO: 119, followed by SEQ ID NO: 124, followed by SEQ ID NO: 123, followed by SEQ ID NO:97, followed by SEQ ID NO:121 (see, e.g., Example 17).

In some cases, a CAR targeting an ENPP1 polypeptide can be designed to include an scFv having a heavy chain variable domain comprising SEQ ID NO:17, SEQ ID NO:18, and SEQ ID NO:19, followed by a linker such as a linker set forth in Example 11, followed by a light chain variable domain comprising SEQ ID NO:25, SEQ ID NO:26, and SEQ ID NO:27, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD3ζ intracellular signaling domain). For example, a CAR targeting an ENPP1 polypeptide can be designed to include an scFv having a heavy chain variable domain comprising SEQ ID NO: 17, SEQ ID NO: 18, and SEQ ID NO: 19, followed by SEQ ID NO: 100, followed by a light chain variable domain comprising SEQ ID NO:25, SEQ ID NO: 26, and SEQ ID NO:27, followed by SEQ ID NO: 102, followed by SEQ ID NO: 108, followed by SEQ ID NO:119, followed by SEQ ID NO: 124, followed by SEQ ID NO: 123, followed by SEQ ID NO:97, followed by SEQ ID NO:121.

In some cases, a CAR targeting an ENPP1 polypeptide can be designed to include an scFv having a heavy chain variable domain comprising SEQ ID NO:24, followed by a linker such as a linker set forth in Example 11, followed by a light chain variable domain comprising SEQ ID NO:32, followed by a hinge such as a hinge/linker set forth in Example 11 or Example 14 (e.g., an IgG4-derived hinge, a CD8 hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD35 intracellular signaling domain). For example, a CAR targeting an ENPP1 polypeptide can be designed to include an scFv having a heavy chain variable domain comprising SEQ ID NO:24, followed by SEQ ID NO:100, followed by a light chain variable domain comprising SEQ ID NO:32, followed by SEQ ID NO: 102, followed by SEQ ID NO: 108, followed by SEQ ID NO: 119, followed by SEQ ID NO: 124, followed by SEQ ID NO: 123, followed by SEQ ID NO:97, followed by SEQ ID NO: 121.

In some cases, a CAR targeting an ENPP1 polypeptide can be designed to include an scFv having a light chain variable domain comprising SEQ ID NO:25, SEQ ID NO:26, and SEQ ID NO:27, followed by a linker such as a linker set forth in Example 11, followed by a heavy chain variable domain comprising SEQ ID NO: 17, SEQ ID NO:18, and SEQ ID NO: 19, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD3 (intracellular signaling domain). For example, a CAR targeting an ENPP1 polypeptide can be designed to include an scFv having a light chain variable domain comprising SEQ ID NO:25, SEQ ID NO:26, and SEQ ID NO:27, followed by SEQ ID NO: 100, followed by a heavy chain variable domain comprising SEQ ID NO:17, SEQ ID NO: 18, and SEQ ID NO: 19, followed by SEQ ID NO: 102, followed by SEQ ID NO: 108, followed by SEQ ID NO: 119, followed by SEQ ID NO: 124, followed by SEQ ID NO: 123, followed by SEQ ID NO:97, followed by SEQ ID NO: 121.

In some cases, a CAR targeting an ENPP1 polypeptide can be designed to include an scFv having a light chain variable domain comprising SEQ ID NO:32, followed by a linker such as a linker set forth in Example 11, followed by a heavy chain variable domain comprising SEQ ID NO:24, followed by a hinge such as a hinge/linker set forth in Example 11 or Example 14 (e.g., an IgG4-derived hinge, a CD8a hinge, or a linker plus IgG4-derived hinge), followed by a transmembrane domain such as a transmembrane domain set forth in Example 15 (e.g., a human CD28 transmembrane domain or a CD8a transmembrane domain), followed by one or more intracellular signaling domains such as one or more intracellular signaling domain set forth in Example 16 (e.g., a human 4-1BB intracellular signaling domain followed by a human CD3ζ intracellular signaling domain). For example, a CAR targeting an ENPP1 polypeptide can be designed to include an scFv having a light chain variable domain comprising SEQ ID NO:32, followed by SEQ ID NO:100, followed by a heavy chain variable domain comprising SEQ ID NO:24, followed by SEQ ID NO:102, followed by SEQ ID NO:108, followed by SEQ ID NO:119, followed by SEQ ID NO: 124, followed by SEQ ID NO:123, followed by SEQ ID NO:97, followed by SEQ ID NO:121. The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, TriKEs, and TriTEs. In general, a cell engager provided herein can be designed to include at least one antigen binding domain having the ability to bind to an ENPP1 polypeptide (e.g., a human ENPP1 polypeptide) and at least one antigen binding domain having the ability to bind to an antigen expressed on the surface of a cell (e.g., a T cell or an NK cell). In some cases, a cell engager described herein can link an ENPP1⁻ cell (e.g., an ENPP1⁺ cancer cell) to another cell (e.g., a T cell or an NK cell) via the two or more antigen binding domains of the cell engager. An example of a cell engager structure of cell engagers provided herein includes, without limitation, the structure set forth in FIG. 9. In some cases, the anti-CD3 scFv depicted in FIG. 9 can be replaced with a different antigen binding domain having the ability to bind to an antigen expressed on the surface of a cell (e.g., a T cell or an NK cell).

When a cell engager includes an antigen binding domain having the ability to bind to an ENPP1 polypeptide (e.g., a human ENPP1 polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to an ENPP1 polypeptide (e.g., a human ENPP1 polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to an ENPP1 polypeptide (e.g., a human ENPP1 polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to an ENPP1 polypeptide (e.g., a human ENPP1 polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs: 1-3 and 9-11 or SEQ ID NOs: 17-19 and 25-27). In some cases, an antigen binding domain of a cell engager targeting an ENPP1 polypeptide can be designed to include a VH domain described herein or a scFv or Fab antibody described herein. In some cases, an antigen binding domain of a CAR described herein that has the ability to bind to an ENPP1 polypeptide (e.g., a human ENPP1 polypeptide) can be used as an antigen binding domain of a cell engager that targets ENPP1⁺ cells.

As described herein, a cell engager can be designed to include at least one antigen binding domain having the ability to bind to an ENPP1 polypeptide (e.g., a human ENPP1 polypeptide) and at least one other antigen binding domain. That at least one other antigen binding domain can have the ability to bind to any appropriate antigen expressed on the surface of a cell. For example, when designing a cell engager such as a BiTE to link an ENPP1⁺ cell and a T cell, the cell engager can include an antigen binding domain having the ability to bind to an ENPP1 polypeptide (e.g., a human ENPP1 polypeptide) and an antigen binding domain having the ability to bind to a polypeptide expressed on the surface of a T cell. Examples example of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

In some cases, a cell engager provided herein can be designed to include an antigen binding domain that comprises, consists essentially of, or consists of one of the amino acid sequences set forth in Example 19. In some cases, a cell engager provided herein can be designed to include an antigen binding domain that comprises, consists essentially of, or consists of one of the amino acid sequences set forth in Example 19 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in Example 19 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link an ENPP1⁺ cell and an NK cell, the cell engager can include an antigen binding domain having the ability to bind to an ENPP1 polypeptide (e.g., a human ENPP1 polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, NKp46 polypeptides, and KIR polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36 (7): 433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

In some cases, a cell engager provided herein can be designed to include an antigen binding domain (e.g., a scFv or VH) that comprises, consists essentially of, or consists of one or more of the amino acid sequences set forth in Example 20. In some cases, a cell engager provided herein can be designed to include an antigen binding domain (e.g., a scFv or VH) that comprises, consists essentially of, or consists of one of the amino acid sequences set forth in Example 20 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in Example 20 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in Example 11. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGGGGGSGGGGS (SEQ ID NO:78) linker. In some cases, a hinge of a CAR described herein can be used as a linker to make a cell engager described herein. For example, any one of the sequences set forth in Example 14 can be used as a linker of a cell engager described herein.

In some cases, a cell engager provided herein can be designed to include a linker that comprises, consists essentially of, or consists of one of the amino acid sequences set forth in Example 11 or Example 14. In some cases, a cell engager provided herein can be designed to include a linker that comprises, consists essentially of, or consists of one of the amino acid sequences set forth in Example 11 or Example 14 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in Example 11 or Example 14 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting an ENPP1 polypeptide can be designed to include an scFv having a heavy chain variable domain comprising SEQ ID NO:1, SEQ ID NO:2, and SEQ ID NO:3, followed by a linker such as a linker set forth in Example 11, followed by a light chain variable domain comprising SEQ ID NO:9, SEQ ID NO:10, and SEQ ID NO:11, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an scFv having a light chain variable domain comprising SEQ ID NO:9, SEQ ID NO:10, and SEQ ID NO:11, followed by a linker such as a linker set forth in Example 11, followed by a heavy chain variable domain comprising SEQ ID NO:1, SEQ ID NO: 2, and SEQ ID NO:3, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an scFv having a heavy chain variable domain comprising SEQ ID NO:8, followed by a linker such as a linker set forth in Example 11, followed by a light chain variable domain comprising SEQ ID NO:16, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an scFv having a light chain variable domain comprising SEQ ID NO:16, followed by a linker such as a linker set forth in Example 11, followed by a heavy chain variable domain comprising SEQ ID NO:8, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiKE or a TriKE) targeting an ENPP1 polypeptide can be designed to include an scFv having a heavy chain variable domain comprising SEQ ID NO:1, SEQ ID NO:2, and SEQ ID NO:3, followed by a linker such as a linker set forth in Example 11, followed by a light chain variable domain comprising SEQ ID NO: 9, SEQ ID NO:10, and SEQ ID NO:11, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an ENPP1 polypeptide can be designed to include an scFv having a light chain variable domain comprising SEQ ID NO:9, SEQ ID NO:10, and SEQ ID NO:11, followed by a linker such as a linker set forth in Example 11, followed by a heavy chain variable domain comprising SEQ ID NO: 1, SEQ ID NO:2, and SEQ ID NO:3, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an ENPP1 polypeptide can be designed to include an scFv having a heavy chain variable domain comprising SEQ ID NO:8, followed by a linker such as a linker set forth in Example 11, followed by a light chain variable domain comprising SEQ ID NO:16, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an ENPP1 polypeptide can be designed to include an scFv having a light chain variable domain comprising SEQ ID NO: 16, followed by a linker such as a linker set forth in Example 11, followed by a heavy chain variable domain comprising SEQ ID NO:8, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an ENPP1 polypeptide can be designed to include an scFv having a heavy chain variable domain comprising SEQ ID NO: 17, SEQ ID NO: 18, and SEQ ID NO: 19, followed by a linker such as a linker set forth in Example 11, followed by a light chain variable domain comprising SEQ ID NO:25, SEQ ID NO: 26, and SEQ ID NO:27, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an scFv having a light chain variable domain comprising SEQ ID NO:25, SEQ ID NO:26, and SEQ ID NO:27, followed by a linker such as a linker set forth in Example 11, followed by a heavy chain variable domain comprising SEQ ID NO:17, SEQ ID NO: 18, and SEQ ID NO: 19, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an scFv having a heavy chain variable domain comprising SEQ ID NO: 24, followed by a linker such as a linker set forth in Example 11, followed by a light chain variable domain comprising SEQ ID NO:32, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an scFv having a light chain variable domain comprising SEQ ID NO:32, followed by a linker such as a linker set forth in Example 11, followed by a heavy chain variable domain comprising SEQ ID NO:24, followed by a linker such as a hinge/linker set forth in Example 11 or Example 14 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiKE or a TriKE) targeting an ENPP1 polypeptide can be designed to include an scFv having a heavy chain variable domain comprising SEQ ID NO:17, SEQ ID NO: 18, and SEQ ID NO:19, followed by a linker such as a linker set forth in Example 11, followed by a light chain variable domain comprising SEQ ID NO:25, SEQ ID NO:26, and SEQ ID NO:27, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an ENPP1 polypeptide can be designed to include an scFv having a light chain variable domain comprising SEQ ID NO:25, SEQ ID NO:26, and SEQ ID NO:27, followed by a linker such as a linker set forth in Example 11, followed by a heavy chain variable domain comprising SEQ ID NO:17, SEQ ID NO:18, and SEQ ID NO: 19, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an ENPP1 polypeptide can be designed to include an scFv having a heavy chain variable domain comprising SEQ ID NO:24, followed by a linker such as a linker set forth in Example 11, followed by a light chain variable domain comprising SEQ ID NO:32, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an ENPP1 polypeptide can be designed to include an scFv having a light chain variable domain comprising SEQ ID NO:32, followed by a linker such as a linker set forth in Example 11, followed by a heavy chain variable domain comprising SEQ ID NO:24, followed by a linker such as a hinge/linker set forth in Example 11 or Example 14 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO: 10, and SEQ ID NO: 11, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO: 10, and SEQ ID NO: 11, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 8, an Ig hinge, and constant domains (e.g., CH1, CH2, and CH3 domains) and (b) a light chain comprising, consisting essentially of, or consisting of a light chain variable domain comprising SEQ ID NO: 16, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiTE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting an ENPP1 polypeptide can be designed to include an IgG (e.g., IgG1) configuration having (a) a heavy chain comprising, consisting essentially of, or consisting of a heavy chain variable domain comprising SEQ ID NO: 24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In one embodiment, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (i) a heavy chain variable domain having a CDR1 having the amino acid sequence set forth in SEQ ID NO:1 (or a variant of SEQ ID NO: 1 with one, two, three, or four amino acid modifications), a CDR2 having the amino acid sequence set forth in SEQ ID NO:2 (or a variant of SEQ ID NO: 2 with one, two, three, or four amino acid modifications), and a CDR3 having the amino acid sequence set forth in SEQ ID NO:3 (or a variant of SEQ ID NO:3 with one, two, three, or four amino acid modifications); and/or (ii) a light chain variable domain having a CDR1 having the amino acid sequence set forth in SEQ ID NO:9 (or a variant of SEQ ID NO:9 with one, two, three, or four amino acid modifications), a CDR2 having the amino acid sequence set forth in SEQ ID NO: 10 (or a variant of SEQ ID NO: 10 with one, two, three, or four amino acid modifications), and a CDR3 having the amino acid sequence set forth SEQ ID NO: 11 (or a variant of SEQ ID NO:11 with one, two, three, or four amino acid modifications). An example of such an antigen binding fragment having these CDRs and the ability to bind to an ENPP1 polypeptide (e.g., a human ENPP1 polypeptide) includes, without limitation, the Fab set forth in Example 3.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) and (a) a heavy chain variable domain having a CDR1 having the amino acid sequence set forth in SEQ ID NO:1 (or a variant of SEQ ID NO: 1 with one, two, three, or four amino acid modifications), a CDR2 having the amino acid sequence set forth in SEQ ID NO:2 (or a variant of SEQ ID NO:2 with one, two, three, or four amino acid modifications), and a CDR3 having the amino acid sequence set forth in SEQ ID NO:3 (or a variant of SEQ ID NO:3 with one, two, three, or four amino acid modifications) and/or (b) a light chain variable domain having a CDR1 having the amino acid sequence set forth in SEQ ID NO:9 (or a variant of SEQ ID NO:9 with one, two, three, or four amino acid modifications), a CDR2 having the amino acid sequence set forth in SEQ ID NO:10 (or a variant of SEQ ID NO: 10 with one, two, three, or four amino acid modifications), and a CDR3 having the amino acid sequence set forth SEQ ID NO: 11 (or a variant of SEQ ID NO:11 with one, two, three, or four amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO: 5 with one, two, three, four, five, six, seven, eight, nine, ten, or more amino acid modifications), a framework region 3 having the amino acid sequence set forth in SEQ ID NO: 6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO: 13 (or a variant of SEQ ID NO: 13 with one, two, three, four, five, six, seven, eight, nine, ten, or more amino acid modifications), a framework region 3 having the amino acid sequence set forth in SEQ ID NO:14 (or a variant of SEQ ID NO: 14 with one, two, three, four, five, six, seven, eight, nine, ten, or more amino acid modifications), and a framework region 4 having the amino acid sequence set forth in SEQ ID NO: 15 (or a variant of SEQ ID NO:15 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in Example 3 can be designed to include framework regions as set forth in Example 3 or can be designed to include one or more framework regions from another antibody, antibody fragment, or antibody domain. For example, an Fab can be designed to include the six CDRs set forth in Example 3 and the framework regions set forth in Example 3 except that framework region 1 having the amino acid set forth in SEQ ID NO:4 is replaced with a framework region 1 having the amino acid set forth in SEQ ID NO:58 or a framework region 1 having the amino acid set forth in SEQ ID NO:67 or a framework region 1 having the amino acid set forth in SEQ ID NO:72. In some cases, a scFv can be designed to include the six CDRs set forth in Example 3 and the framework regions set forth in Example 3. In some cases, a scFv can be designed to include the six CDRs set forth in Example 3 and the framework regions set forth in Example 3 except that framework region 1 having the amino acid set forth in SEQ ID NO: 4 is replaced with a framework region 1 having the amino acid set forth in SEQ ID NO: 58, a framework region 1 having the amino acid set forth in SEQ ID NO:67, or a framework region 1 having the amino acid set forth in SEQ ID NO:72. In another example, a scFv can be designed to include the six CDRs set forth in Example 3 and the framework regions set forth in Example 7, Example 8, Example 9, or Example 10.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO: 16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs: 9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO: 16, provided that the light chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO: 16 or the amino acid set forth in SEQ ID NO: 16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an ENPP1 polypeptide (e.g., a human ENPP1 polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 1, 2, and 3, and can include a light chain variable domain having the amino acid sequence set forth in SEQ ID NO: 16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO: 9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO: 11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR1 that consists essentially of the amino acid sequence set forth in SEQ ID NO: 1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| GFEFSEYY | 166 |
| GFTFSEYY | 167 |
| GFEFSDYY | 168 |
| GFGYSDYY | 169 |
| GFTFSKEY | 170 |
| GFTFAEYY | 171 |
| GFTFTDYY | 172 |
| GFTFSNYY | 173 |
| GFTLSDYY | 174 |
| YFTFSDYY | 175 |

As used herein, a “CDR2 that consists essentially of the amino acid sequence set forth in SEQ ID NO:2” is a CDR2 that has zero, one, or two amino acid substitutions within SEQ ID NO: 2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| ITSSGSTV | 176 |
| ITSSGSTI | 177 |
| INSSGSTI | 178 |
| ISSSGSTV | 179 |
| ISNSGSTI | 180 |
| VSSSGSTI | 181 |
| ISSSGTTI | 182 |
| IASSGSTI | 183 |
| ISSAGSTI | 184 |
| ISSSGNTI | 185 |

As used herein, a “CDR3 that consists essentially of the amino acid sequence set forth in SEQ ID NO:3” is a CDR3 that has zero, one, or two amino acid substitutions within SEQ ID NO: 3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR3 that consists essentially of the amino acid sequence set forth in SEQ ID NO:3 include, without limitation, those set forth in Table 3.

TABLE 3
Exemplary CDR3s that consist essentially of
the amino acid sequence set forth in
SEQ ID NO: 3.

| Sequence | SEQ ID NO: |
|---|---|
| AREVYYYDPTGYYGYYFNY | 186 |
| ARDVYYYDPTGYYGYYFNY | 187 |
| ARQIYYYGHDGYYEYIFDY | 188 |
| ASEVYYYDSSGYYTYYFDY | 189 |
| ARLVYFYDSSGYYTYYFDY | 190 |
| AREVYYYDSSPYYQEYFDY | 191 |
| ARQIYYYDSSGYYTYYFDY | 192 |
| AREVYYYGHDGYYTYYFDY | 193 |
| AREVYYYDSSGYYEYIFDY | 194 |
| ARQVYYYGHDGYYTYYFDY | 195 |

As used herein, a “CDR1 that consists essentially of the amino acid sequence set forth in SEQ ID NO:9” is a CDR1 that has zero, one, or two amino acid substitutions within SEQ ID NO: 9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR1 that consists essentially of the amino acid sequence set forth in SEQ ID NO:9 include, without limitation, those set forth in Table 4.

TABLE 4
Exemplary CDR1s that consist essentially of
the amino acid sequence set forth in
SEQ ID NO: 9.

| Sequence | SEQ ID NO: |
|---|---|
| QTVASY | 196 |
| QSVSSY | 197 |
| QSISTY | 198 |
| QSISNY | 199 |
| QSITSY | 200 |
| QSISAY | 201 |
| QSINSY | 202 |
| QTISSY | 203 |
| QSIASY | 204 |
| QSVASY | 205 |

As used herein, a “CDR2 that consists essentially of the amino acid sequence set forth in SEQ ID NO:10” is a CDR2 that has zero, one, or two amino acid substitutions within SEQ ID NO: 10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR2 that consists essentially of the amino acid sequence set forth in SEQ ID NO:10 include, without limitation, those set forth in Table 5.

TABLE 5
Exemplary CDR2s that consist essentially of
the amino acid sequence set forth in
SEQ ID NO: 10.

| Sequence | SEQ ID NO: |
|---|---|
| AATSLQS | 206 |
| AASSMQS | 207 |
| AASALQS | 208 |
| AASGLQS | 209 |
| AASTLQS | 210 |
| AATSMQS | 211 |
| AATALQS | 212 |
| AATGLQS | 213 |
| AATTLQS | 214 |
| AASAMQS | 215 |

As used herein, a “CDR3 that consists essentially of the amino acid sequence set forth in SEQ ID NO:11” is a CDR3 that has zero, one, or two amino acid substitutions within SEQ ID NO: 11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR3 that consists essentially of the amino acid sequence set forth in SEQ ID NO:11 include, without limitation, those set forth in Table 6.

TABLE 6
Exemplary CDR3s that consist essentially of
the amino acid sequence set forth in
SEQ ID NO: 11.

| Sequence | SEQ ID NO: |
|---|---|
| QQYGTFPLT | 216 |
| QQYDSFPLT | 217 |
| QQYNTFPLT | 218 |
| QQYEPFPLT | 219 |
| QKYDTFPLT | 220 |
| QPYDTFPLT | 221 |
| QEYDPFPLT | 222 |
| QAYDTFPLT | 223 |
| QQYDNFPLT | 224 |
| QQYETFPLT | 225 |

In another embodiment, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (i) a heavy chain variable domain having a CDR1 having the amino acid sequence set forth in SEQ ID NO: 17 (or a variant of SEQ ID NO:17 with one, two, three, or four amino acid modifications), a CDR2 having the amino acid sequence set forth in SEQ ID NO:18 (or a variant of SEQ ID NO:18 with one, two, three, or four amino acid modifications), and a CDR3 having the amino acid sequence set forth in SEQ ID NO:19 (or a variant of SEQ ID NO: 19 with one, two, three, or four amino acid modifications); and/or (ii) a light chain variable domain having a CDR1 having the amino acid sequence set forth in SEQ ID NO:25 (or a variant of SEQ ID NO:25 with one, two, three, or four amino acid modifications), a CDR2 having the amino acid sequence set forth in SEQ ID NO:26 (or a variant of SEQ ID NO: 26 with one, two, three, or four amino acid modifications), and a CDR3 having the amino acid sequence set forth SEQ ID NO:27 (or a variant of SEQ ID NO:27 with one, two, three, or four amino acid modifications). An example of such an antigen binding fragment having these CDRs and the ability to bind to an ENPP1 polypeptide (e.g., a human ENPP1 polypeptide) includes, without limitation, the Fab set forth in Example 4.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) and having (a) a heavy chain variable domain having a CDR1 having the amino acid sequence set forth in SEQ ID NO:17 (or a variant of SEQ ID NO:17 with one, two, three, or four amino acid modifications), a CDR2 having the amino acid sequence set forth in SEQ ID NO:18 (or a variant of SEQ ID NO: 18 with one, two, three, or four amino acid modifications), and a CDR3 having the amino acid sequence set forth in SEQ ID NO:19 (or a variant of SEQ ID NO: 19 with one, two, three, or four amino acid modifications) and/or (b) a light chain variable domain having a CDR1 having the amino acid sequence set forth in SEQ ID NO:25 (or a variant of SEQ ID NO: 25 with one, two, three, or four amino acid modifications), a CDR2 having the amino acid sequence set forth in SEQ ID NO:26 (or a variant of SEQ ID NO:26 with one, two, three, or four amino acid modifications), and a CDR3 having the amino acid sequence set forth SEQ ID NO:27 (or a variant of SEQ ID NO:27 with one, two, three, or four amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO: 21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO: 23 with one, two, three, four, five, six, seven, eight, nine, ten, or more amino acid modifications) and/or (b) a light chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO: 29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO: 31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in Example 4 can be designed to include framework regions as set forth in Example 4 or can be designed to include one or more framework regions from another antibody or antibody fragment. For example, an Fab can be designed to include the six CDRs set forth in Example 4 and the framework regions set forth in Example 4 except that framework region 1 having the amino acid set forth in SEQ ID NO:20 is replaced with a framework region 1 having the amino acid set forth in SEQ ID NO:58 or a framework region 1 having the amino acid set forth in SEQ ID NO: 67 or a framework region 1 having the amino acid set forth in SEQ ID NO:72. In some cases, a scFv can be designed to include the six CDRs set forth in Example 4 and the framework regions set forth in Example 4. In some cases, a scFv can be designed to include the six CDRs set forth in Example 4 and the framework regions set forth in Example 4 except that framework region 1 having the amino acid set forth in SEQ ID NO:20 is replaced with a framework region 1 having the amino acid set forth in SEQ ID NO:58, a framework region 1 having the amino acid set forth in SEQ ID NO:67, or a framework region 1 having the amino acid set forth in SEQ ID NO:72. In another example, a scFv can be designed to include the six CDRs set forth in Example 4 and the framework regions set forth in Example 7, Example 8, Example 9, or Example 10.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs: 17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs: 25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO: 24, provided that the heavy chain variable domain includes the amino acid sequences set forth in SEQ ID NOs: 17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO:32, provided that the light chain variable domain includes the amino acid sequences set forth in SEQ ID NOs: 25, 26, and 27.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an ENPP1 polypeptide (e.g., a human ENPP1 polypeptide), can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 25, 26, and 27.

In some cases, a binder (e.g., an antibody, antigen binding fragment, antibody domain, a CAR, a cell engager, and/or an ADC) provided herein having the ability to bind to an ENPP1 polypeptide (e.g., a human ENPP1 polypeptide) can include (a) a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO: 18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO: 25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO: 17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR1 that consists essentially of the amino acid sequence set forth in SEQ ID NO: 17 include, without limitation, those set forth in Table 7.

TABLE 7
Exemplary CDR1s that consist essentially of
the amino acid sequence set forth in
SEQ ID NO: 17.

| Sequence | SEQ ID NO: |
|---|---|
| GYTFSEYF | 226 |
| GYTFSEYY | 227 |
| GFRFSDYY | 228 |
| GYTFNDYY | 229 |
| GFTFSDHY | 230 |
| GFSFSEYY | 231 |
| GFTFNDYF | 232 |
| GFTFPNYY | 233 |
| GFTFTDFY | 234 |
| GFTCSDYY | 235 |

As used herein, a “CDR2 that consists essentially of the amino acid sequence set forth in SEQ ID NO:18” is a CDR2 that has zero, one, or two amino acid substitutions within SEQ ID NO: 18, that has zero, one, two, three, four, or five amino acid residues directly preceding SEQ ID NO:18, and/or that has zero, one, two, three, four, or five amino acid residues directly following SEQ ID NO:18, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR2 that consists essentially of the amino acid sequence set forth in SEQ ID NO:18 include, without limitation, those set forth in Table 8.

TABLE 8
Exemplary CDR2s that consist essentially of
the amino acid sequence set forth in
SEQ ID NO: 18.

| Sequence | SEQ ID NO: |
|---|---|
| ISASGTTI | 236 |
| ISSSGTTI | 237 |
| ISASGSTI | 238 |
| ISSSGATI | 239 |
| ISSTGSTI | 240 |
| ISSSGSSI | 241 |
| ISASGETI | 242 |
| ISSSDSTI | 243 |
| IPSSGSTI | 244 |
| ISGSGSTI | 245 |

As used herein, a “CDR3 that consists essentially of the amino acid sequence set forth in SEQ ID NO:19” is a CDR3 that has zero, one, or two amino acid substitutions within SEQ ID NO: 19, that has zero, one, two, three, four, or five amino acid residues directly preceding SEQ ID NO:19, and/or that has zero, one, two, three, four, or five amino acid residues directly following SEQ ID NO: 19, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR3 that consists essentially of the amino acid sequence set forth in SEQ ID NO:19 include, without limitation, those set forth in Table 9.

TABLE 9
Exemplary CDR3s that consist essentially of
the amino acid sequence set forth in
SEQ ID NO: 19.

| Sequence | SEQ ID NO: |
|---|---|
| AREIYYYGHSGYYTYYFDY | 246 |
| ARQIYYYGHSGYYTYYFDY | 247 |
| ARQIYYYDHDGYYTYYFDY | 248 |
| AREIYYYGHDGYYEYYFDY | 249 |
| ARDVYYYDSSGYYGYYFNY | 250 |
| AREVYYYDPTGYYTYYFDY | 251 |
| ARDVYYYDPSGYYGYYFNY | 252 |
| AREVYYYDSSGYYGYYFNY | 253 |
| AREVYYYEGSGYYTYYFGY | 254 |
| ARDVYYYDSSGYYTYYFNY | 255 |

As used herein, a “CDR1 that consists essentially of the amino acid sequence set forth in SEQ ID NO:25” is a CDR1 that has zero, one, or two amino acid substitutions within SEQ ID NO: 25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR1 that consists essentially of the amino acid sequence set forth in SEQ ID NO:25 include, without limitation, those set forth in Table 10.

TABLE 10
Exemplary CDR1s that consist essentially of
the amino acid sequence set forth in
SEQ ID NO: 25.

| Sequence | SEQ ID NO: |
|---|---|
| QSVNAY | 256 |
| QSVSTY | 257 |
| QSVSNY | 258 |
| QSVTNY | 259 |
| QSVNSY | 260 |
| QSVAAY | 261 |
| QTINSY | 262 |
| QTVSSY | 263 |
| QTIASY | 264 |
| QTVASY | 265 |

As used herein, a “CDR2 that consists essentially of the amino acid sequence set forth in SEQ ID NO:26” is a CDR2 that has zero, one, or two amino acid substitutions within SEQ ID NO: 26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR2 that consists essentially of the amino acid sequence set forth in SEQ ID NO:26 include, without limitation, those set forth in Table 11.

TABLE 11
Exemplary CDR2s that consist essentially of
the amino acid sequence set forth in
SEQ ID NO: 26.

| Sequence | SEQ ID NO: |
|---|---|
| AASGMQS | 266 |
| AASTMQS | 267 |
| AATAMQS | 268 |
| AATGMQS | 269 |
| AATTMQS | 270 |
| AATSLQS | 271 |
| AASSMQS | 272 |
| AATSMQS | 273 |
| AASAMQS | 274 |
| AASTMQS | 275 |

As used herein, a “CDR3 that consists essentially of the amino acid sequence set forth in SEQ ID NO:27” is a CDR3 that has zero, one, or two amino acid substitutions within SEQ ID NO: 27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to an ENPP1 polypeptide (e.g., a human ENPP1 polypeptide). Examples of a CDR3 that consists essentially of the amino acid sequence set forth in SEQ ID NO:27 include, without limitation, those set forth in Table 12.

TABLE 12
Exemplary CDR3s that consist essentially of
the amino acid sequence set forth in
SEQ ID NO: 27.

| Sequence | SEQ ID NO: |
|---|---|
| QQFYTTRIT | 276 |
| QQYYTSGIT | 277 |
| QQYYSSQIT | 278 |
| QQYYTERIT | 279 |
| QQYYTARIT | 280 |
| QEYYTFRIT | 281 |
| QQYYLNRIT | 282 |
| QQYYTDHIT | 283 |
| QQYYTGPIT | 284 |
| QEYFTARIT | 285 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs: 1-3 or SEQ ID NOs: 17-19 can be directly connected to a light chain variable domain having the CDRs of SEQ ID NOs: 9-11 or SEQ ID NOs: 25-27, respectively, via a linker sequence. Examples of linker sequences that can be used to connect a heavy chain variable domain and a light chain variable domain to create a scFv include, without limitation, those linkers set forth in Example 11.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to an ENPP1 polypeptide (e.g., a human ENPP1 polypeptide) are further described in Table 13.

TABLE 13
Representative number of binders.

| | SEQ ID NOs of | SEQ ID NOs of | | SEQ ID NOs of | SEQ ID NOs of | |
|---|---|---|---|---|---|---|
| | Heavy Chain | Heavy Chain | SEQ ID NO of | Light Chain | Light Chain | |
| | Variable | Variable | Heavy Chain | Variable | Variable | SEQ ID NO of |
| Clone # | Domain/ | Domain/Region | Variable | Domain/ | Domain/Region | Light Chain |
| (Antibody | Region | Framework | Domain/ | Region | Framework | Variable |
| type) | CDRs | Regions | Region | CDRs | Regions | Domain/Region |
| #1 (Fab) | 1, 2, 3 | 4, 5, 6, 7 | 8 | 9, 10, 11 | 12, 13, 14, 15 | 16 |
| #2 (Fab) | 17, 18, 19 | 20, 21, 22, 23 | 24 | 25, 26, 27 | 28, 29, 30, 31 | 32 |

Table 14 includes an alternative designation that can be used to refer to each of Clones #1-#2.

TABLE 14
Alternative nomenclature for Clones #1-#2.

| Clone # | Alternative names |
|---|---|
| 1 | 17-Fab |
| 2 | 3G12-Fab |

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. Example 5 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae/casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of an ENPP1 polypeptide (e.g., a human ENPP1 polypeptide). In some cases, a bispecific binder provided herein can be designed to bind to two different epitopes of the same ENPP1 polypeptide (e.g., a human ENPP1 polypeptide). In some cases, a bispecific binder provided herein can bind to an ENPP1 polypeptide (e.g., a human ENPP1 polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9 (2): 182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to an ENPP1 polypeptide (e.g., a human ENPP1 polypeptide) and (b) at least one drug covalently linked directly or indirectly to that antigen binding domain. Any appropriate binder (e.g., an antibody, antigen binding fragment, and/or antibody domain) provided herein and having the ability to bind to an ENPP1 polypeptide (e.g., a human ENPP1 polypeptide) can be used as an antigen binding domain to make an ADC described herein. For example, any of the binders set forth in Table 13 can be used to make an ADC having the ability to bind to an ENPP1 polypeptide (e.g., a human ENPP1 polypeptide). Examples of drugs that can be used to make an ADC described herein include, without limitation, auristatins (e.g., monomethyl auristatin E (MMAE)), mertansine (DM-1), pyrrolobenzodiazepine (PBD) dimers, ravtansine (DM-4), and monomethyl auristatin F (MMAF). Any appropriate ADC linker can be used to covalently attach one or more drugs to an antigen binding domain having the ability to bind to an ENPP1 polypeptide (e.g., a human ENPP1 polypeptide) to form an ADC provided herein. For example, cleavable or non-cleavable ADC linkers can be used to covalently attach one or more drugs to an antigen binding domain having the ability to bind to an ENPP1 polypeptide (e.g., a human ENPP1 polypeptide) to form an ADC provided herein. Examples of ADC linkers can be used to covalently attach one or more drugs to an antigen binding domain having the ability to bind to an ENPP1 polypeptide (e.g., a human ENPP1 polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in Example 3 or Example 4. In another example, an isolated nucleic acid molecule provided herein can include a nucleic acid sequence encoding a light chain variable domain such as a light chain variable domain as set forth in Example 3 or Example 4. In some cases, an isolated nucleic acid molecule provided herein can include a nucleic acid sequence encoding both (a) a heavy chain variable domain and (b) a light chain variable domain, with or without, encoding a linker polypeptide set forth in Example 11. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors, and fowlpox virus-based vectors), and hybrid or chimeric viral vectors. For example, a viral vector having an adenoviral backbone with lentiviral components such as those described elsewhere (Zheng et al., Nat. Biotech., 18 (2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N. Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukayotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to an ENPP1 polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to an ENPP1 polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer (e.g., one or more sarcomas) to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to an ENPP1 polypeptide (e.g., a human ENPP1 polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to an ENPP1 polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to an ENPP1 polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰). In some cases, a pharmaceutical composition containing one or more cells designed to express a CAR having the ability to bind to an ENPP1 polypeptide provided herein can be formulated as a dosage form with from about 1.5×10⁵ cells/kg anti-ENPP1 CAR⁺ cells to about 10¹⁰ cells/kg anti-ENPP1 CAR⁺ cells (e.g., from 3×10⁵ cells/kg to about 10¹⁰ cells/kg, from 5×10⁵ cells/kg to about 10¹⁰ cells/kg, from 10⁶ cells/kg to about 10¹⁰ cells/kg, from 1.5×10⁶ cells/kg to about 10⁹ cells/kg, from 10⁷ cells/kg to about 10⁸ cells/kg, or from 10⁸ cells/kg to about 10⁹ cells/kg of anti-ENPP1 CAR⁺ cells). In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer (e.g., one or more sarcomas) to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) having cancer (e.g., a ENPP1⁺ cancer) to reduce or eliminate one or more symptoms of the cancer. Examples of symptoms of a cancer (e.g., a sarcoma) that can be reduced using a composition comprising one or more binders described herein include, without limitation, one or more lumps that can be felt through the skin, bone pain, abdominal pain, and weight loss.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. In some cases, a cancer that can be treated as described herein can include one or more solid tumors. In some cases, a cancer that can be treated as described herein can be a blood cancer. In some cases, a cancer that can be treated as described herein can be a sarcoma (e.g., a soft tissue sarcoma or a bone cancer). Examples of cancers that can be treated as described herein include, without limitation, angiosarcomas, chondrosarcomas, dermatofibrosarcoma protuberans, desmoplastic small round cell tumors, epithelioid sarcomas, Ewing sarcomas, gastrointestinal stromal tumor (gist), Kaposi's sarcomas, leiomyosarcomas, liposarcomas, malignant peripheral nerve sheath tumors, myxofibrosarcomas, osteosarcomas, rhabdomyosarcomas, soft tissue sarcomas, solitary fibrous tumosr, synovial sarcomas, undifferentiated pleomorphic sarcomas, lung cancers, breast cancers, and ovarian cancers. In some cases, a mammal (e.g., a human) having a ENPP1⁺ cancer (e.g., an ENPP1⁺ sarcoma, an ENPP1⁺ lung cancer, an ENPP1⁺ breast cancer, or an ENPP1⁺ ovarian cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer (e.g., one or more sarcomas) without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer (e.g., one or more sarcomas) as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). In another example, an effective amount of cells (e.g., T cells) expressing a binder (e.g., CAR) provided herein can be from about 1.5×10⁵ anti-ENPP1 CAR⁺ cells/kg of body weight to about 10¹⁰ anti-ENPP1 CAR⁺ cells/kg of body weight (e.g., from 3×10⁵ cells/kg to about 10⁹ cells/kg, from 1⁰⁶ cells/kg to about 5×10⁸ cells/kg, from 5×10⁶ cells/kg to about 10⁸ cells/kg, from 10⁷ cells/kg to about 5×10⁷ cells/kg, from 5×10⁷ cells/kg to about 10⁸ cells/kg, or from 10⁸ cells/kg to about 10⁹ cells/kg). The effective amount can remain constant or can be adjusted as a sliding scale or variable dose depending on the mammal's response to treatment. Various factors can influence the actual effective amount used for a particular application. For example, the severity of cancer when treating a mammal having cancer (e.g., one or more sarcomas), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer (e.g., one or more sarcomas) without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer (e.g., one or more sarcomas) as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer (e.g., one or more sarcomas), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer (e.g., one or more sarcomas) as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer (e.g., one or more sarcomas), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of an ENPP1 polypeptide (e.g., a human ENPP1 polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of an ENPP1 polypeptide (e.g., a human ENPP1 polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express an ENPP1 polypeptide (e.g., a human ENPP1 polypeptide). In some cases, a binder (e.g., an antibody, antigen binding fragment, and/or antibody domain) provided herein can be immobilized, e.g., on a support, and retention of an ENPP1 polypeptide (e.g., a human ENPP1 polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of an ENPP1 polypeptide (e.g., a human ENPP1 polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### Examples

### Example 1: Human Antibody Fragments Targeting ENPP1

This Example describes the design and characterization of molecules that can bind to an ENPP1 polypeptide.

### Materials and Method:

Expression and Purification of ENPP1, IgG1, Fab and scFv

ENPP1 extracellular domain was synthesized by IDT (Coralville, IA, USA) and cloned into a pIW-Zeocin expression vector designed to express the ENPP1 extracellular domain fused with a His6-FLAG tag. For the conversion of IgG1 from Fab, the heavy chain and light chain of the Fab were amplified and re-cloned into the pcDNA-IgG1 vector. For transient expression of ENPP1-His and IgG1, the plasmid was transfected into Expi293 cells by PEI, and purified by Nickel resin (GE Healthcare, Chicago, IL, USA) and Protein A resin (GenScript, Piscataway, NJ, USA) separately. The expression and purification of Fab and scFv binders were performed in E. coli Top 10F′ bacterial with 1 mM IPTG induction at 30° C. for 16 hours. Bacterial pellets were lysed by Polymyxin B (Sigma-Aldrich, St. Louis, MO, USA), and the supernatant was loaded on Ni-NTA column for purification.

### Amino Acid Sequences of ENPP1 Binders:

| | | SEQ |
|---|---|---|
| | | ID |
| Binder | Amino Acid Sequence | NO |
| Fab 17 | Fab 17-Light chain | 286 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDAATYYCQQYDTFPLTFGGGTKVE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| | Fab 17-Heavy chain | 287 |
| | QVQLVESGGGLVKPGGSLRLSCAASGFTFSDYYMS | |
| | WIRQAPGKGLEWVSYISSSGSTIYYADSVKGRFTI | |
| | SRDNAKNSLYLQMNSLRAEDTAVYYCAREVYYYDS | |
| | SGYYTYYFDYWGQGTLVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKT | |
| Fab 3G12 | Fab 3G12-Light chain | 288 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDVAVYYCQQYYTSRITFGQGTKLE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| | Fab 3G12-Heavy chain | 287 |
| | QVQLVESGGGLVKPGGSLRLSCAASGFTFSDYYMS | |
| | WIRQAPGKGLEWVSYISSSGSTIYYADSVKGRFTI | |
| | SRDNAKNSLYLQMNSLRAEDTAVYYCAREVYYYDS | |
| | SGYYTYYFDYWGQGTLVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKT | |
| IgG1 17 | IgG1 17 HC | 289 |
| | QVQLVESGGGLVKPGGSLRLSCAASGFTFSDYYMS | |
| | WIRQAPGKGLEWVSYISSSGSTIYYADSVKGRFTI | |
| | SRDNAKNSLYLQMNSLRAEDTAVYYCAREVYYYDS | |
| | SGYYTYYFDYWGQGTLVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSRDELTKNQVSLTCLVKGFYPSD | |
| | IAVEWESNGQPENNYKTTPPVLDSDGSFFLYSKLT | |
| | VDKSRWQQGNVFSCSVMHEALHNHYTQKSLSLSPG | |
| | K | |
| | IgG1 17 LC | 286 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDAATYYCQQYDTFPLTFGGGTKVE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| IgG1 3G12 | IgG1 3G12 HC | 289 |
| | QVQLVESGGGLVKPGGSLRLSCAASGFTFSDYYMS | |
| | WIRQAPGKGLEWVSYISSSGSTIYYADSVKGRFTI | |
| | SRDNAKNSLYLQMNSLRAEDTAVYYCAREVYYYDS | |
| | SGYYTYYFDYWGQGTLVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSRDELTKNQVSLTCLVKGFYPSD | |
| | IAVEWESNGQPENNYKTTPPVLDSDGSFFLYSKLT | |
| | VDKSRWQQGNVFSCSVMHEALHNHYTQKSLSLSPG | |
| | K | |
| | IgG1 3G12 LC | 288 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDVAVYYCQQYYTSRITFGQGTKLE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| scFv 17 | DIQMTQSPSSLSASVGDRVTITCRASQSISSYLNW | 290 |
| | YQQKPGKAPKLLIYAASSLQSGVPSRFSGSGSGTD | |
| | FTLTISSLQPEDAATYYCQQYDTFPLTFGGGTKVE | |
| | IKGGGGSGGGGSGGGGSQVQLVESGGGLVKPGGSL | |
| | RLSCAASGFTFSDYYMSWIRQAPGKGLEWVSYISS | |
| | SGSTIYYADSVKGRFTISRDNAKNSLYLQMNSLRA | |
| | EDTAVYYCAREVYYYDSSGYYTYYFDYWGQGTLVT | |
| | VSS | |
| scFv 3G12 | DIQMTQSPSSLSASVGDRVTITCRASQSISSYLNW | 291 |
| | YQQKPGKAPKLLIYAASSLQSGVPSRFSGSGSGTD | |
| | FTLTISSLQPEDVAVYYCQQYYTSRITFGQGTKLE | |
| | IKGGGGSGGGGSGGGGSQVQLVESGGGLVKPGGSL | |
| | RLSCAASGFTFSDYYMSWIRQAPGKGLEWVSYISS | |
| | SGSTIYYADSVKGRFTISRDNAKNSLYLQMNSLRA | |
| | EDTAVYYCAREVYYYDSSGYYTYYFDYWGQGTLVT | |
| | VSS | |
| CAR 17 | scFv 17 | 290 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDAATYYCQQYDTFPLTFGGGTKVE | |
| | IKGGGGSGGGGSGGGGSQVQLVESGGGLVKPGGSL | |
| | RLSCAASGFTFSDYYMSWIRQAPGKGLEWVSYISS | |
| | SGSTIYYADSVKGRFTISRDNAKNSLYLQMNSLRA | |
| | EDTAVYYCAREVYYYDSSGYYTYYFDYWGQGTLVT | |
| | VSS | |
| | CAR chain | 292 |
| | GQAGRTTTPAPRPPTPAPTIASQPLSLRPEACRPA | |
| | AGGAVHTRGLDFACDIYIWAPLAGTCGVLLLSLVI | |
| | TLYCKRGRKKLLYIFKQPFMRPVQTTQEEDGCSCR | |
| | FPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLS | |
| | TATKDTYDALHMQALPPR | |
| CAR 3G12 | scFv 3G12 | 291 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDVAVYYCQQYYTSRITFGQGTKLE | |
| | IKGGGGSGGGGSGGGGSQVQLVESGGGLVKPGGSL | |
| | RLSCAASGFTFSDYYMSWIRQAPGKGLEWVSYISS | |
| | SGSTIYYADSVKGRFTISRDNAKNSLYLQMNSLRA | |
| | EDTAVYYCAREVYYYDSSGYYTYYFDYWGQGTLVT | |
| | VSS | |
| | CAR chain | 292 |
| | GQAGRTTTPAPRPPTPAPTIASQPLSLRPEACRPA | |
| | AGGAVHTRGLDFACDIYIWAPLAGTCGVLLLSLVI | |
| | TLYCKRGRKKLLYIFKQPFMRPVQTTQEEDGCSCR | |
| | FPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLS | |
| | TATKDTYDALHMQALPPR | |
| BiTE 17 | BiTE 17 HC | 289 |
| | QVQLVESGGGLVKPGGSLRLSCAASGFTFSDYYMS | |
| | WIRQAPGKGLEWVSYISSSGSTIYYADSVKGRFTI | |
| | SRDNAKNSLYLQMNSLRAEDTAVYYCAREVYYYDS | |
| | SGYYTYYFDYWGQGTLVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSRDELTKNQVSLTCLVKGFYPSD | |
| | IAVEWESNGQPENNYKTTPPVLDSDGSFFLYSKLT | |
| | VDKSRWQQGNVFSCSVMHEALHNHYTQKSLSLSPG | |
| | K | |
| | BiTE 17 LC | 293 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDAATYYCQQYDTFPLTFGGGTKVE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGECGGGGSGGGGSGGGGSQVQLVQSGGGVVQPGR | |
| | SLRLSCKASGYTFTRYTMHWVRQAPGKGLEWIGYI | |
| | NPSRGYTNYNQKVKDRFTISRDNSKNTAFLQMDSL | |
| | RPEDTGVYFCARYYDDHYCLDYWGQGTPVTVSSGG | |
| | GGSGGGGSGGGGSGGGGSGGGGSGGGGSDIQMTQS | |
| | PSSLSASVGDRVTITCSASSSVSYMNWYQQTPGKA | |
| | PKRWIYDTSKLASGVPSRFSGSGSGTDYTFTISSL | |
| | QPEDIATYYCQQWSSNPFTFGQGTKLQITR | |
| BiTE 3G12 | BiTE 3G12 HC | 289 |
| | QVQLVESGGGLVKPGGSLRLSCAASGFTFSDYYMS | |
| | WIRQAPGKGLEWVSYISSSGSTIYYADSVKGRFTI | |
| | SRDNAKNSLYLQMNSLRAEDTAVYYCAREVYYYDS | |
| | SGYYTYYFDYWGQGTLVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSRDELTKNQVSLTCLVKGFYPSD | |
| | IAVEWESNGQPENNYKTTPPVLDSDGSFFLYSKLT | |
| | VDKSRWQQGNVFSCSVMHEALHNHYTQKSLSLSPG | |
| | K | |
| | BiTE 3G12 LC | 294 |
| | DIQMTQSPSSLSASVGDRVTITCRASQSISSYLNW | |
| | YQQKPGKAPKLLIYAASSLQSGVPSRFSGSGSGTD | |
| | FTLTISSLQPEDVAVYYCQQYYTSRITFGQGTKLE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGECGGGGSGGGGSGGGGSQVQLVQSGGGVVQPGR | |
| | SLRLSCKASGYTFTRYTMHWVRQAPGKGLEWIGYI | |
| | NPSRGYTNYNQKVKDRFTISRDNSKNTAFLQMDSL | |
| | RPEDTGVYFCARYYDDHYCLDYWGQGTPVTVSSGG | |
| | GGSGGGGSGGGGSGGGGSGGGGSGGGGSDIQMTQS | |
| | PSSLSASVGDRVTITCSASSSVSYMNWYQQTPGKA | |
| | PKRWIYDTSKLASGVPSRFSGSGSGTDYTFTISSL | |
| | QPEDIATYYCQQWSSNPFTFGQGTKLQITR | |

### Nucleic Acid Sequences Encoding ENPP1 Binders:

| Binder | Nucleic Acid Sequence | SEQ ID NO |
|---|---|---|
| Fab 17 | Fab 17-Light chain: | 295 |
| | GATATTCAAATGACCCAGTCTCCTTCTTCCTTGAGCGCGTCTGTG | |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGCTGCAACGTATTACTGTCAACAA | |
| | TATGATACTTTTCCACTCACTTTCGGCGGAGGGACCAAAGTGGAG | |
| | ATCAAACGTACGGTGGCTGCTCCGTCGGTGTTTATCTTTCCACCG | |
| | AGTGACGAACAACTGAAGTCGGGGACTGCCAGCGTTGTTTGCCTT | |
| | CTGAACAATTTTTACCCCCGCGAGGCGAAGGTTCAGTGGAAAGTC | |
| | GATAATGCGTTACAGAGCGGGAATAGCCAGGAAAGTGTTACTGAA | |
| | CAGGATAGTAAAGACAGCACTTACAGCCTCAGCAGCACCCTGACG | |
| | CTGAGCAAAGCAGACTACGAAAAACATAAAGTCTACGCCTGCGAA | |
| | GTCACCCATCAGGGCCTGAGCTCGCCCGTCACAAAGAGCTTCAAC | |
| | AGGGGAGAGTGT | |
| | Fab 17-Heavy chain: | 297 |
| | CAAGTTCAATTAGTGGAGAGCGGCGGCGGATTGGTTAAACCGGGG | |
| | GGCAGTCTGCGTTTAAGCTGCGCAGCTTCCGGGTTTACTTTTTCG | |
| | GATTACTATATGAGCTGGATTCGTCAGGCTCCCGGTAAAGGGTTG | |
| | GAATGGGTTAGCTACATCTCAAGTTCCGGCTCTACAATCTACTAT | |
| | GCAGATTCCGTGAAAGGGCGCTTCACCATCTCTCGTGACAATGCA | |
| | AAGAACAGTTTATACTTACAAATGAACTCGCTGCGTGCGGAAGAT | |
| | ACGGCCGTGTATTACTGTGCGAGAGAGGTATATTACTATGATAGT | |
| | AGTGGTTATTATACCTACTACTTTGACTACTGGGGCCAGGGAACC | |
| | CTGGTCACTGTCTCCTCAGCGTCGACCAAAGGGCCCAGTGTCTTT | |
| | CCGTTAGCTCCGTCTTCCAAAAGTACGTCGGGCGGCACGGCGGCA | |
| | CTTGGCTGCTTAGTCAAGGACTACTTTCCCGAACCCGTTACTGTC | |
| | TCTTGGAATAGTGGGGCTTTGACCAGTGGGGTTCATACCTTTCCA | |
| | GCCGTACTTCAGTCTTCAGGGCTGTATAGTTTAAGTTCAGTGGTT | |
| | ACAGTGCCCTCCAGCAGCTTGGGCACCCAGACCTACATCTGCAAC | |
| | GTGAATCACAAGCCCAGCAACACCAAGGTCGACAAGAAAGTTGAG | |
| | CCCAAATCTTGTGACAAAACT | |
| Fab 3G12 | Fab 3G12-Light chain: | 298 |
| | GATATTCAAATGACCCAGTCTCCTTCTTCCTTGAGCGCGTCTGTG | |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGTGGCAGTTTATTACTGTCAACAA | |
| | TATTACACTTCTCGTATCACGTTCGGCCAAGGGACCAAGCTGGAA | |
| | ATCAAACGTACGGTGGCTGCTCCGTCGGTGTTTATCTTTCCACCG | |
| | AGTGACGAACAACTGAAGTCGGGGACTGCCAGCGTTGTTTGCCTT | |
| | CTGAACAATTTTTACCCCCGCGAGGCGAAGGTTCAGTGGAAAGTC | |
| | GATAATGCGTTACAGAGCGGGAATAGCCAGGAAAGTGTTACTGAA | |
| | CAGGATAGTAAAGACAGCACTTACAGCCTCAGCAGCACCCTGACG | |
| | CTGAGCAAAGCAGACTACGAAAAACATAAAGTCTACGCCTGCGAA | |
| | GTCACCCATCAGGGCCTGAGCTCGCCCGTCACAAAGAGCTTCAAC | |
| | AGGGGAGAGTGT | |
| | Fab 3G12-Heavy chain: | 297 |
| | CAAGTTCAATTAGTGGAGAGCGGCGGCGGATTGGTTAAACCGGGG | |
| | GGCAGTCTGCGTTTAAGCTGCGCAGCTTCCGGGTTTACTTTTTCG | |
| | GATTACTATATGAGCTGGATTCGTCAGGCTCCCGGTAAAGGGTTG | |
| | GAATGGGTTAGCTACATCTCAAGTTCCGGCTCTACAATCTACTAT | |
| | GCAGATTCCGTGAAAGGGCGCTTCACCATCTCTCGTGACAATGCA | |
| | AAGAACAGTTTATACTTACAAATGAACTCGCTGCGTGCGGAAGAT | |
| | ACGGCCGTGTATTACTGTGCGAGAGAGGTATATTACTATGATAGT | |
| | AGTGGTTATTATACCTACTACTTTGACTACTGGGGCCAGGGAACC | |
| | CTGGTCACTGTCTCCTCAGCGTCGACCAAAGGGCCCAGTGTCTTT | |
| | CCGTTAGCTCCGTCTTCCAAAAGTACGTCGGGCGGCACGGCGGCA | |
| | CTTGGCTGCTTAGTCAAGGACTACTTTCCCGAACCCGTTACTGTC | |
| | TCTTGGAATAGTGGGGCTTTGACCAGTGGGGTTCATACCTTTCCA | |
| | GCCGTACTTCAGTCTTCAGGGCTGTATAGTTTAAGTTCAGTGGTT | |
| | ACAGTGCCCTCCAGCAGCTTGGGCACCCAGACCTACATCTGCAAC | |
| | GTGAATCACAAGCCCAGCAACACCAAGGTCGACAAGAAAGTTGAG | |
| | CCCAAATCTTGTGACAAAACT | |
| IgG1 17 | IgG1 17 HC: | 296 |
| | CAAGTTCAATTAGTGGAGAGCGGCGGCGGATTGGTTAAACCGGGG | |
| | GGCAGTCTGCGTTTAAGCTGCGCAGCTTCCGGGTTTACTTTTTCG | |
| | GATTACTATATGAGCTGGATTCGTCAGGCTCCCGGTAAAGGGTTG | |
| | GAATGGGTTAGCTACATCTCAAGTTCCGGCTCTACAATCTACTAT | |
| | GCAGATTCCGTGAAAGGGCGCTTCACCATCTCTCGTGACAATGCA | |
| | AAGAACAGTTTATACTTACAAATGAACTCGCTGCGTGCGGAAGAT | |
| | ACGGCCGTGTATTACTGTGCGAGAGAGGTATATTACTATGATAGT | |
| | AGTGGTTATTATACCTACTACTTTGACTACTGGGGCCAGGGAACC | |
| | CTGGTCACTGTCTCCTCAGCGTCGACCAAAGGGCCCAGTGTCTTT | |
| | CCGTTAGCTCCGTCTTCCAAAAGTACGTCGGGCGGCACGGCGGCA | |
| | CTTGGCTGCTTAGTCAAGGACTACTTTCCCGAACCCGTTACTGTC | |
| | TCTTGGAATAGTGGGGCTTTGACCAGTGGGGTTCATACCTTTCCA | |
| | GCCGTACTTCAGTCTTCAGGGCTGTATAGTTTAAGTTCAGTGGTT | |
| | ACAGTGCCCTCCAGCAGCTTGGGCACCCAGACCTACATCTGCAAC | |
| | GTGAATCACAAGCCCAGCAACACCAAGGTCGACAAGAAAGTTGAG | |
| | CCCAAATCTTGTGACAAAACTCACACATGCCCACCGTGCCCAGCA | |
| | CCTGAACTCCTGGGGGGACCGTCAGTCTTCCTCTTCCCCCCAAAA | |
| | CCCAAGGACACCCTCATGATCTCCCGGACCCCTGAGGTCACATGC | |
| | GTGGTGGTGGACGTGAGCCACGAAGACCCTGAGGTCAAGTTCAAC | |
| | TGGTACGTGGACGGCGTGGAGGTGCATAATGCCAAGACAAAGCCG | |
| | CGGGAGGAGCAGTACAACAGCACGTACCGTGTGGTCAGCGTCCTC | |
| | ACCGTCCTGCACCAGGACTGGCTGAATGGCAAGGAGTACAAGTGC | |
| | AAGGTCTCCAACAAAGCCCTCCCAGCCCCCATCGAGAAAACCATC | |
| | TCCAAAGCCAAAGGGCAGCCCCGAGAACCACAGGTGTACACCCTG | |
| | CCCCCATCCCGGGATGAGCTGACCAAGAACCAGGTCAGCCTGACC | |
| | TGCCTGGTCAAAGGCTTCTATCCCAGCGACATCGCCGTGGAGTGG | |
| | GAGAGCAATGGGCAGCCGGAGAACAACTACAAGACCACACCTCCC | |
| | GTGCTGGACTCCGACGGCTCCTTCTTCCTCTACAGCAAGCTCACC | |
| | GTGGACAAGAGCAGGTGGCAGCAGGGGAACGTCTTCTCATGCTCC | |
| | GTGATGCATGAGGCTCTGCACAACCACTACACGCAGAAGAGCCTC | |
| | TCCCTGTCCCCGGGTAAATAG | |
| | IgG1 17 LC: | |
| | GATATTCAAATGACCCAGTCTCCTTCTTCCTTGAGCGCGTCTGTG | 295 |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGCTGCAACGTATTACTGTCAACAA | |
| | TATGATACTTTTCCACTCACTTTCGGCGGAGGGACCAAAGTGGAG | |
| | ATCAAACGTACGGTGGCTGCTCCGTCGGTGTTTATCTTTCCACCG | |
| | AGTGACGAACAACTGAAGTCGGGGACTGCCAGCGTTGTTTGCCTT | |
| | CTGAACAATTTTTACCCCCGCGAGGCGAAGGTTCAGTGGAAAGTC | |
| | GATAATGCGTTACAGAGCGGGAATAGCCAGGAAAGTGTTACTGAA | |
| | CAGGATAGTAAAGACAGCACTTACAGCCTCAGCAGCACCCTGACG | |
| | CTGAGCAAAGCAGACTACGAAAAACATAAAGTCTACGCCTGCGAA | |
| | GTCACCCATCAGGGCCTGAGCTCGCCCGTCACAAAGAGCTTCAAC | |
| | AGGGGAGAGTGT | |
| IgG1 3G12 | IgG1 3G12 HC: | 296 |
| | CAAGTTCAATTAGTGGAGAGCGGCGGCGGATTGGTTAAACCGGGG | |
| | GGCAGTCTGCGTTTAAGCTGCGCAGCTTCCGGGTTTACTTTTTCG | |
| | GATTACTATATGAGCTGGATTCGTCAGGCTCCCGGTAAAGGGTTG | |
| | GAATGGGTTAGCTACATCTCAAGTTCCGGCTCTACAATCTACTAT | |
| | GCAGATTCCGTGAAAGGGCGCTTCACCATCTCTCGTGACAATGCA | |
| | AAGAACAGTTTATACTTACAAATGAACTCGCTGCGTGCGGAAGAT | |
| | ACGGCCGTGTATTACTGTGCGAGAGAGGTATATTACTATGATAGT | |
| | AGTGGTTATTATACCTACTACTTTGACTACTGGGGCCAGGGAACC | |
| | CTGGTCACTGTCTCCTCAGCGTCGACCAAAGGGCCCAGTGTCTTT | |
| | CCGTTAGCTCCGTCTTCCAAAAGTACGTCGGGCGGCACGGCGGCA | |
| | CTTGGCTGCTTAGTCAAGGACTACTTTCCCGAACCCGTTACTGTC | |
| | TCTTGGAATAGTGGGGCTTTGACCAGTGGGGTTCATACCTTTCCA | |
| | GCCGTACTTCAGTCTTCAGGGCTGTATAGTTTAAGTTCAGTGGTT | |
| | ACAGTGCCCTCCAGCAGCTTGGGCACCCAGACCTACATCTGCAAC | |
| | GTGAATCACAAGCCCAGCAACACCAAGGTCGACAAGAAAGTTGAG | |
| | CCCAAATCTTGTGACAAAACTCACACATGCCCACCGTGCCCAGCA | |
| | CCTGAACTCCTGGGGGGACCGTCAGTCTTCCTCTTCCCCCCAAAA | |
| | CCCAAGGACACCCTCATGATCTCCCGGACCCCTGAGGTCACATGC | |
| | GTGGTGGTGGACGTGAGCCACGAAGACCCTGAGGTCAAGTTCAAC | |
| | TGGTACGTGGACGGCGTGGAGGTGCATAATGCCAAGACAAAGCCG | |
| | CGGGAGGAGCAGTACAACAGCACGTACCGTGTGGTCAGCGTCCTC | |
| | ACCGTCCTGCACCAGGACTGGCTGAATGGCAAGGAGTACAAGTGC | |
| | AAGGTCTCCAACAAAGCCCTCCCAGCCCCCATCGAGAAAACCATC | |
| | TCCAAAGCCAAAGGGCAGCCCCGAGAACCACAGGTGTACACCCTG | |
| | CCCCCATCCCGGGATGAGCTGACCAAGAACCAGGTCAGCCTGACC | |
| | TGCCTGGTCAAAGGCTTCTATCCCAGCGACATCGCCGTGGAGTGG | |
| | GAGAGCAATGGGCAGCCGGAGAACAACTACAAGACCACACCTCCC | |
| | GTGCTGGACTCCGACGGCTCCTTCTTCCTCTACAGCAAGCTCACC | |
| | GTGGACAAGAGCAGGTGGCAGCAGGGGAACGTCTTCTCATGCTCC | |
| | GTGATGCATGAGGCTCTGCACAACCACTACACGCAGAAGAGCCTC | |
| | TCCCTGTCCCCGGGTAAATAG | |
| | IgG1 3G12 LC: | |
| | GATATTCAAATGACCCAGTCTCCTTCTTCCTTGAGCGCGTCTGTG | 298 |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGTGGCAGTTTATTACTGTCAACAA | |
| | TATTACACTTCTCGTATCACGTTCGGCCAAGGGACCAAGCTGGAA | |
| | ATCAAACGTACGGTGGCTGCTCCGTCGGTGTTTATCTTTCCACCG | |
| | AGTGACGAACAACTGAAGTCGGGGACTGCCAGCGTTGTTTGCCTT | |
| | CTGAACAATTTTTACCCCCGCGAGGCGAAGGTTCAGTGGAAAGTC | |
| | GATAATGCGTTACAGAGCGGGAATAGCCAGGAAAGTGTTACTGAA | |
| | CAGGATAGTAAAGACAGCACTTACAGCCTCAGCAGCACCCTGACG | |
| | CTGAGCAAAGCAGACTACGAAAAACATAAAGTCTACGCCTGCGAA | |
| | GTCACCCATCAGGGCCTGAGCTCGCCCGTCACAAAGAGCTTCAAC | |
| | AGGGGAGAGTGT | |
| scFv 17 | GATATTCAAATGACCCAGTCTCCTTCTTCCTTGAGCGCGTCTGTG | 299 |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGCTGCAACGTATTACTGTCAACAA | |
| | TATGATACTTTTCCACTCACTTTCGGCGGAGGGACCAAAGTGGAG | |
| | ATCAAAGGAGGGGGTGGCAGCGGTGGCGGGGGAAGTGGCGGTGGA | |
| | GGGTCGCAAGTTCAATTAGTGGAGAGCGGCGGCGGATTGGTTAAA | |
| | CCGGGGGGCAGTCTGCGTTTAAGCTGCGCAGCTTCCGGGTTTACT | |
| | TTTTCGGATTACTATATGAGCTGGATTCGTCAGGCTCCCGGTAAA | |
| | GGGTTGGAATGGGTTAGCTACATCTCAAGTTCCGGCTCTACAATC | |
| | TACTATGCAGATTCCGTGAAAGGGCGCTTCACCATCTCTCGTGAC | |
| | AATGCAAAGAACAGTTTATACTTACAAATGAACTCGCTGCGTGCG | |
| | GAAGATACGGCCGTGTATTACTGTGCGAGAGAGGTATATTACTAT | |
| | GATAGTAGTGGTTATTATACCTACTACTTTGACTACTGGGGCCAG | |
| | GGAACCCTGGTCACTGTCTCCTCA | |
| scFv 3G12 | GATATTCAAATGACCCAGTCTCCTTCTTCCTTGAGCGCGTCTGTG | 300 |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGTGGCAGTTTATTACTGTCAACAA | |
| | TATTACACTTCTCGTATCACGTTCGGCCAAGGGACCAAGCTGGAA | |
| | ATCAAAGGAGGGGGTGGCAGCGGTGGCGGGGGAAGTGGCGGTGGA | |
| | GGGTCGCAAGTTCAATTAGTGGAGAGCGGCGGCGGATTGGTTAAA | |
| | CCGGGGGGCAGTCTGCGTTTAAGCTGCGCAGCTTCCGGGTTTACT | |
| | TTTTCGGATTACTATATGAGCTGGATTCGTCAGGCTCCCGGTAAA | |
| | GGGTTGGAATGGGTTAGCTACATCTCAAGTTCCGGCTCTACAATC | |
| | TACTATGCAGATTCCGTGAAAGGGCGCTTCACCATCTCTCGTGAC | |
| | AATGCAAAGAACAGTTTATACTTACAAATGAACTCGCTGCGTGCG | |
| | GAAGATACGGCCGTGTATTACTGTGCGAGAGAGGTATATTACTAT | |
| | GATAGTAGTGGTTATTATACCTACTACTTTGACTACTGGGGCCAG | |
| | GGAACCCTGGTCACTGTCTCCTCA | |
| CAR 17 | scFv 17: | 299 |
| | GATATTCAAATGACCCAGTCTCCTTCTTCCTTGAGCGCGTCTGTG | |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGCTGCAACGTATTACTGTCAACAA | |
| | TATGATACTTTTCCACTCACTTTCGGCGGAGGGACCAAAGTGGAG | |
| | ATCAAAGGAGGGGGTGGCAGCGGTGGCGGGGGAAGTGGCGGTGGA | |
| | GGGTCGCAAGTTCAATTAGTGGAGAGCGGCGGCGGATTGGTTAAA | |
| | CCGGGGGGCAGTCTGCGTTTAAGCTGCGCAGCTTCCGGGTTTACT | |
| | TTTTCGGATTACTATATGAGCTGGATTCGTCAGGCTCCCGGTAAA | |
| | GGGTTGGAATGGGTTAGCTACATCTCAAGTTCCGGCTCTACAATC | |
| | TACTATGCAGATTCCGTGAAAGGGCGCTTCACCATCTCTCGTGAC | |
| | AATGCAAAGAACAGTTTATACTTACAAATGAACTCGCTGCGTGCG | |
| | GAAGATACGGCCGTGTATTACTGTGCGAGAGAGGTATATTACTAT | |
| | GATAGTAGTGGTTATTATACCTACTACTTTGACTACTGGGGCCAG | |
| | GGAACCCTGGTCACTGTCTCCTCA | |
| | CAR chain: | 301 |
| | GGCCAGGCCGGCCGCACCACGACGCCAGCGCCGCGACCACCAACA | |
| | CCGGCGCCCACCATCGCGTCGCAGCCCCTGTCCCTGCGCCCAGAG | |
| | GCGTGCCGGCCAGCGGCGGGGGGCGCAGTGCACACGAGGGGGCTG | |
| | GACTTCGCCTGTGATATCTACATCTGGGCGCCCTTGGCCGGGACT | |
| | TGTGGGGTCCTTCTCCTGTCACTGGTTATCACCCTTTACTGCAAA | |
| | CGGGGCAGAAAGAAACTCCTGTATATATTCAAACAACCATTTATG | |
| | AGACCAGTACAAACTACTCAAGAGGAAGATGGCTGTAGCTGCCGA | |
| | TTTCCAGAAGAAGAAGAAGGAGGATGTGAACTGAGAGTGAAGTTC | |
| | AGCAGGAGCGCAGACGCCCCCGCGTACAAGCAGGGCCAGAACCAG | |
| | CTCTATAACGAGCTCAATCTAGGACGAAGAGAGGAGTACGATGTT | |
| | TTGGACAAGAGACGTGGCCGGGACCCTGAGATGGGGGGAAAGCCG | |
| | AGAAGGAAGAACCCTCAGGAAGGCCTGTACAATGAACTGCAGAAA | |
| | GATAAGATGGCGGAGGCCTACAGTGAGATTGGGATGAAAGGCGAG | |
| | CGCCGGAGGGGCAAGGGGCACGATGGCCTTTACCAGGGTCTCAGT | |
| | ACAGCCACCAAGGACACCTACGACGCCCTTCACATGCAGGCCCTG | |
| | CCCCCTCGCTAA | |
| CAR 3G12 | scFv 3G12: | |
| | GATATTCAAATGACCCAGTCTCCTTCTTCCTTGAGCGCGTCTGTG | 300 |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGTGGCAGTTTATTACTGTCAACAA | |
| | TATTACACTTCTCGTATCACGTTCGGCCAAGGGACCAAGCTGGAA | |
| | ATCAAAGGAGGGGGTGGCAGCGGTGGCGGGGGAAGTGGCGGTGGA | |
| | GGGTCGCAAGTTCAATTAGTGGAGAGCGGCGGCGGATTGGTTAAA | |
| | CCGGGGGGCAGTCTGCGTTTAAGCTGCGCAGCTTCCGGGTTTACT | |
| | TTTTCGGATTACTATATGAGCTGGATTCGTCAGGCTCCCGGTAAA | |
| | GGGTTGGAATGGGTTAGCTACATCTCAAGTTCCGGCTCTACAATC | |
| | TACTATGCAGATTCCGTGAAAGGGCGCTTCACCATCTCTCGTGAC | |
| | AATGCAAAGAACAGTTTATACTTACAAATGAACTCGCTGCGTGCG | |
| | GAAGATACGGCCGTGTATTACTGTGCGAGAGAGGTATATTACTAT | |
| | GATAGTAGTGGTTATTATACCTACTACTTTGACTACTGGGGCCAG | |
| | GGAACCCTGGTCACTGTCTCCTCA | |
| | CAR chain: | 301 |
| | GGCCAGGCCGGCCGCACCACGACGCCAGCGCCGCGACCACCAACA | |
| | CCGGCGCCCACCATCGCGTCGCAGCCCCTGTCCCTGCGCCCAGAG | |
| | GCGTGCCGGCCAGCGGCGGGGGGCGCAGTGCACACGAGGGGGCTG | |
| | GACTTCGCCTGTGATATCTACATCTGGGCGCCCTTGGCCGGGACT | |
| | TGTGGGGTCCTTCTCCTGTCACTGGTTATCACCCTTTACTGCAAA | |
| | CGGGGCAGAAAGAAACTCCTGTATATATTCAAACAACCATTTATG | |
| | AGACCAGTACAAACTACTCAAGAGGAAGATGGCTGTAGCTGCCGA | |
| | TTTCCAGAAGAAGAAGAAGGAGGATGTGAACTGAGAGTGAAGTTC | |
| | AGCAGGAGCGCAGACGCCCCCGCGTACAAGCAGGGCCAGAACCAG | |
| | CTCTATAACGAGCTCAATCTAGGACGAAGAGAGGAGTACGATGTT | |
| | TTGGACAAGAGACGTGGCCGGGACCCTGAGATGGGGGGAAAGCCG | |
| | AGAAGGAAGAACCCTCAGGAAGGCCTGTACAATGAACTGCAGAAA | |
| | GATAAGATGGCGGAGGCCTACAGTGAGATTGGGATGAAAGGCGAG | |
| | CGCCGGAGGGGCAAGGGGCACGATGGCCTTTACCAGGGTCTCAGT | |
| | ACAGCCACCAAGGACACCTACGACGCCCTTCACATGCAGGCCCTG | |
| | CCCCCTCGCTAA | |
| BiTE 17 | BiTE 17 HC: | |
| | CAAGTTCAATTAGTGGAGAGCGGCGGCGGATTGGTTAAACCGGGG | 302 |
| | GGCAGTCTGCGTTTAAGCTGCGCAGCTTCCGGGTTTACTTTTTCG | |
| | GATTACTATATGAGCTGGATTCGTCAGGCTCCCGGTAAAGGGTTG | |
| | GAATGGGTTAGCTACATCTCAAGTTCCGGCTCTACAATCTACTAT | |
| | GCAGATTCCGTGAAAGGGCGCTTCACCATCTCTCGTGACAATGCA | |
| | AAGAACAGTTTATACTTACAAATGAACTCGCTGCGTGCGGAAGAT | |
| | ACGGCCGTGTATTACTGTGCGAGAGAGGTATATTACTATGATAGT | |
| | AGTGGTTATTATACCTACTACTTTGACTACTGGGGCCAGGGAACC | |
| | CTGGTCACTGTCTCCTCAGCGTCGACCAAAGGGCCCAGTGTCTTT | |
| | CCGTTAGCTCCGTCTTCCAAAAGTACGTCGGGCGGCACGGCGGCA | |
| | CTTGGCTGCTTAGTCAAGGACTACTTTCCCGAACCCGTTACTGTC | |
| | TCTTGGAATAGTGGGGCTTTGACCAGTGGGGTTCATACCTTTCCA | |
| | GCCGTACTTCAGTCTTCAGGGCTGTATAGTTTAAGTTCAGTGGTT | |
| | ACAGTGCCCTCCAGCAGCTTGGGCACCCAGACCTACATCTGCAAC | |
| | GTGAATCACAAGCCCAGCAACACCAAGGTCGACAAGAAAGTTGAG | |
| | CCCAAATCTTGTGACAAAACTCACACATGCCCACCGTGCCCAGCA | |
| | CCTGAACTCCTGGGGGGACCGTCAGTCTTCCTCTTCCCCCCAAAA | |
| | CCCAAGGACACCCTCATGATCTCCCGGACCCCTGAGGTCACATGC | |
| | GTGGTGGTGGACGTGAGCCACGAAGACCCTGAGGTCAAGTTCAAC | |
| | TGGTACGTGGACGGCGTGGAGGTGCATAATGCCAAGACAAAGCCG | |
| | CGGGAGGAGCAGTACAACAGCACGTACCGTGTGGTCAGCGTCCTC | |
| | ACCGTCCTGCACCAGGACTGGCTGAATGGCAAGGAGTACAAGTGC | |
| | AAGGTCTCCAACAAAGCCCTCCCAGCCCCCATCGAGAAAACCATC | |
| | TCCAAAGCCAAAGGGCAGCCCCGAGAACCACAGGTGTACACCCTG | |
| | CCCCCATCCCGGGATGAGCTGACCAAGAACCAGGTCAGCCTGACC | |
| | TGCCTGGTCAAAGGCTTCTATCCCAGCGACATCGCCGTGGAGTGG | |
| | GAGAGCAATGGGCAGCCGGAGAACAACTACAAGACCACACCTCCC | |
| | GTGCTGGACTCCGACGGCTCCTTCTTCCTCTACAGCAAGCTCACC | |
| | GTGGACAAGAGCAGGTGGCAGCAGGGGAACGTCTTCTCATGCTCC | |
| | GTGATGCATGAGGCTCTGCACAACCACTACACGCAGAAGAGCCTC | |
| | TCCCTGTCCCCGGGTAAATAG | |
| | BiTE 17 LC: | |
| | GATATTCAAATGACCCAGTCTCCTTCTTCCTTGAGCGCGTCTGTG | 303 |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGCTGCAACGTATTACTGTCAACAA | |
| | TATGATACTTTTCCACTCACTTTCGGCGGAGGGACCAAAGTGGAG | |
| | ATCAAACGTACGGTGGCTGCTCCGTCGGTGTTTATCTTTCCACCG | |
| | AGTGACGAACAACTGAAGTCGGGGACTGCCAGCGTTGTTTGCCTT | |
| | CTGAACAATTTTTACCCCCGCGAGGCGAAGGTTCAGTGGAAAGTC | |
| | GATAATGCGTTACAGAGCGGGAATAGCCAGGAAAGTGTTACTGAA | |
| | CAGGATAGTAAAGACAGCACTTACAGCCTCAGCAGCACCCTGACG | |
| | CTGAGCAAAGCAGACTACGAAAAACATAAAGTCTACGCCTGCGAA | |
| | GTCACCCATCAGGGCCTGAGCTCGCCCGTCACAAAGAGCTTCAAC | |
| | AGGGGAGAGTGTGGAGGTGGCGGGTCTGGTGGAGGCGGAAGCGGT | |
| | GGTGGCGGATCCCAGGTCCAACTGGTGCAAAGTGGAGGCGGCGTA | |
| | GTACAGCCCGGTCGGTCCCTGCGGCTCAGCTGTAAAGCAAGTGGG | |
| | TACACTTTCACCAGATATACAATGCACTGGGTTAGACAGGCCCCC | |
| | GGCAAGGGTCTTGAATGGATAGGATATATCAACCCATCCCGCGGA | |
| | TATACAAACTACAATCAGAAGGTGAAGGACCGGTTCACGATTAGT | |
| | AGAGACAACTCCAAAAACACTGCGTTCCTTCAAATGGACTCATTG | |
| | CGACCCGAGGACACTGGAGTGTATTTCTGTGCCAGGTATTATGAC | |
| | GACCATTATTGCCTGGACTACTGGGGGCAAGGCACACCGGTTACG | |
| | GTATCTTCCGGAGGTGGCGGGTCTGGTGGAGGCGGAAGCGGTGGT | |
| | GGCGGATCCGGGGGAGGAGGCTCTGGCGGAGGCGGATCAGGGGGT | |
| | GGAGGTTCCGACATTCAAATGACACAATCCCCTAGTTCTTTGTCT | |
| | GCGTCCGTGGGGGATCGGGTAACAATCACTTGCTCTGCATCAAGC | |
| | AGTGTTTCTTATATGAACTGGTATCAGCAGACACCCGGTAAGGCC | |
| | CCAAAACGATGGATTTATGACACGAGTAAACTCGCATCCGGGGTG | |
| | CCTTCACGCTTCTCCGGTTCTGGTTCCGGCACGGATTACACTTTC | |
| | ACCATCAGTAGCCTGCAACCTGAGGACATTGCAACTTATTACTGT | |
| | CAACAATGGAGCAGTAATCCATTCACGTTCGGCCAAGGGACAAAA | |
| | CTGCAGATTACTAGGTAA | |
| BiTE 3G12 | BiTE 3G12 HC: | 302 |
| | CAAGTTCAATTAGTGGAGAGCGGCGGCGGATTGGTTAAACCGGGGG | |
| | GCAGTCTGCGTTTAAGCTGCGCAGCTTCCGGGTTTACTTTTTCGG | |
| | ATTACTATATGAGCTGGATTCGTCAGGCTCCCGGTAAAGGGTTGG | |
| | AATGGGTTAGCTACATCTCAAGTTCCGGCTCTACAATCTACTATG | |
| | CAGATTCCGTGAAAGGGCGCTTCACCATCTCTCGTGACAATGCAA | |
| | AGAACAGTTTATACTTACAAATGAACTCGCTGCGTGCGGAAGATA | |
| | CGGCCGTGTATTACTGTGCGAGAGAGGTATATTACTATGATAGTA | |
| | GTGGTTATTATACCTACTACTTTGACTACTGGGGCCAGGGAACCC | |
| | TGGTCACTGTCTCCTCAGCGTCGACCAAAGGGCCCAGTGTCTTTC | |
| | CGTTAGCTCCGTCTTCCAAAAGTACGTCGGGCGGCACGGCGGCAC | |
| | TTGGCTGCTTAGTCAAGGACTACTTTCCCGAACCCGTTACTGTCT | |
| | CTTGGAATAGTGGGGCTTTGACCAGTGGGGTTCATACCTTTCCAG | |
| | CCGTACTTCAGTCTTCAGGGCTGTATAGTTTAAGTTCAGTGGTTA | |
| | CAGTGCCCTCCAGCAGCTTGGGCACCCAGACCTACATCTGCAACG | |
| | TGAATCACAAGCCCAGCAACACCAAGGTCGACAAGAAAGTTGAGC | |
| | CCAAATCTTGTGACAAAACTCACACATGCCCACCGTGCCCAGCAC | |
| | CTGAACTCCTGGGGGGACCGTCAGTCTTCCTCTTCCCCCCAAAAC | |
| | CCAAGGACACCCTCATGATCTCCCGGACCCCTGAGGTCACATGCG | |
| | TGGTGGTGGACGTGAGCCACGAAGACCCTGAGGTCAAGTTCAACT | |
| | GGTACGTGGACGGCGTGGAGGTGCATAATGCCAAGACAAAGCCGC | |
| | GGGAGGAGCAGTACAACAGCACGTACCGTGTGGTCAGCGTCCTCA | |
| | CCGTCCTGCACCAGGACTGGCTGAATGGCAAGGAGTACAAGTGCA | |
| | AGGTCTCCAACAAAGCCCTCCCAGCCCCCATCGAGAAAACCATCT | |
| | CCAAAGCCAAAGGGCAGCCCCGAGAACCACAGGTGTACACCCTGC | |
| | CCCCATCCCGGGATGAGCTGACCAAGAACCAGGTCAGCCTGACCT | |
| | GCCTGGTCAAAGGCTTCTATCCCAGCGACATCGCCGTGGAGTGGG | |
| | AGAGCAATGGGCAGCCGGAGAACAACTACAAGACCACACCTCCCG | |
| | TGCTGGACTCCGACGGCTCCTTCTTCCTCTACAGCAAGCTCACCG | |
| | TGGACAAGAGCAGGTGGCAGCAGGGGAACGTCTTCTCATGCTCCG | |
| | TGATGCATGAGGCTCTGCACAACCACTACACGCAGAAGAGCCTCT | |
| | CCCTGTCCCCGGGTAAATAGG | |
| | BiTE 3G12 LC: | 304 |
| | GATATTCAAATGACCCAGTCTCCTTCTTCCTTGAGCGCGTCTGTG | |
| | GGAGACCGTGTAACTATCACGTGTCGTGCATCCCAAAGCATTTCG | |
| | AGTTACTTGAACTGGTATCAACAAAAGCCTGGTAAAGCGCCCAAA | |
| | CTGTTGATCTATGCTGCCAGTAGTCTTCAATCTGGTGTGCCCTCG | |
| | CGCTTCTCAGGTTCGGGGTCAGGAACGGACTTTACCCTGACCATT | |
| | AGCTCTCTGCAACCGGAAGATGTGGCAGTTTATTACTGTCAACAA | |
| | TATTACACTTCTCGTATCACGTTCGGCCAAGGGACCAAGCTGGAA | |
| | ATCAAACGTACGGTGGCTGCTCCGTCGGTGTTTATCTTTCCACCG | |
| | AGTGACGAACAACTGAAGTCGGGGACTGCCAGCGTTGTTTGCCTT | |
| | CTGAACAATTTTTACCCCCGCGAGGCGAAGGTTCAGTGGAAAGTC | |
| | GATAATGCGTTACAGAGCGGGAATAGCCAGGAAAGTGTTACTGAA | |
| | CAGGATAGTAAAGACAGCACTTACAGCCTCAGCAGCACCCTGACG | |
| | CTGAGCAAAGCAGACTACGAAAAACATAAAGTCTACGCCTGCGAA | |
| | GTCACCCATCAGGGCCTGAGCTCGCCCGTCACAAAGAGCTTCAAC | |
| | AGGGGAGAGTGTGGAGGTGGCGGGTCTGGTGGAGGCGGAAGCGGT | |
| | GGTGGCGGATCCCAGGTCCAACTGGTGCAAAGTGGAGGCGGCGTA | |
| | GTACAGCCCGGTCGGTCCCTGCGGCTCAGCTGTAAAGCAAGTGGG | |
| | TACACTTTCACCAGATATACAATGCACTGGGTTAGACAGGCCCCC | |
| | GGCAAGGGTCTTGAATGGATAGGATATATCAACCCATCCCGCGGA | |
| | TATACAAACTACAATCAGAAGGTGAAGGACCGGTTCACGATTAGT | |
| | AGAGACAACTCCAAAAACACTGCGTTCCTTCAAATGGACTCATTG | |
| | CGACCCGAGGACACTGGAGTGTATTTCTGTGCCAGGTATTATGAC | |
| | GACCATTATTGCCTGGACTACTGGGGGCAAGGCACACCGGTTACG | |
| | GTATCTTCCGGAGGTGGCGGGTCTGGTGGAGGCGGAAGCGGTGGT | |
| | GGCGGATCCGGGGGAGGAGGCTCTGGCGGAGGCGGATCAGGGGGT | |
| | GGAGGTTCCGACATTCAAATGACACAATCCCCTAGTTCTTTGTCT | |
| | GCGTCCGTGGGGGATCGGGTAACAATCACTTGCTCTGCATCAAGC | |
| | AGTGTTTCTTATATGAACTGGTATCAGCAGACACCCGGTAAGGCC | |
| | CCAAAACGATGGATTTATGACACGAGTAAACTCGCATCCGGGGTG | |
| | CCTTCACGCTTCTCCGGTTCTGGTTCCGGCACGGATTACACTTTC | |
| | ACCATCAGTAGCCTGCAACCTGAGGACATTGCAACTTATTACTGT | |
| | CAACAATGGAGCAGTAATCCATTCACGTTCGGCCAAGGGACAAAA | |
| | CTGCAGATTACTAGGTAA | |

Panning from Large Fab Phage Library

To pan antibodies against ENPP1, a large Fab phage library was used. Briefly, ENPP1-His protein was biotinylated by using a biotin conjugation kit (Abcam, Cambridge, UK) according to the manufacturer's instruction. The phage library was first incubated with streptavidin-coated M-280 Dyna beads (Invitrogen, Waltham, MA, USA) at room temperature for 20 minutes for nonspecific phage deletion. Blocking phage library was performed using 5% milk for 1 hour at room temperature, and then the library was incubated with 5 μg biotinylated ENPP1-His. Bound phages were separated by streptavidin beads by incubating at room temperature for 1 hour, and then they were washed using PBS. After washing, phages were eluted using 0.1 M of PH3 Glycine and were neutralized with 1 M of Tris-HCl (pH8). For the second and third round panning, 2.5 μg and 1.25 μg of biotinylated ENPP1-His were used as antigens, respectively. After the third round of panning, 192 individual clones were analyzed by ELISA.

### Size-Exclusion Chromatography (SEC)

The purity and structure of the antibodies were analyzed by Superdex 200 Increase 10/300 GL chromatography (GE Healthcare, Chicago, IL, USA). Briefly, the proteins ferritin, aldolase, conalbumin, ovalbumin, carbonic anhydrase, and ribonuclease were used for calibration. A 500 μL sample mix containing the above proteins was loaded into the column and separated by the ÄKTA explorer machine (GE Healthcare, Chicago, IL, USA). For the antibody analysis, 100 μL of filtered antibodies (2 mg/mL) in 1×DPBS (Dulbecco's phosphate-buffered saline, Gibco, Waltham, MA, USA) was analyzed. Antibodies were eluted by DPBS buffer at a flow rate of 0.5 mL/minute.

### Enzyme-Linked Immunosorbent Assay (ELISA)

The binding and specificity of Fab, scFv, and IgG1 to ENPP1 were analyzed by ELISA. Briefly, ENPP1-His protein was coated at 50 ng/well at 4° C. overnight and blocked with 5% milk for 2 hours at room temperature. After three times washing with PBST, they were incubated with 3-fold serially diluted Fab, scFv, or IgG1 for 1 hour at 37° C. For Fab and scFv detection via the FLAG tag present at the C-terminus, further incubation was performed with anti-FLAG M2-peroxidase (HRP) antibody (A8592, Sigma-Aldrich, St. Louis, MO, USA) for another 1 hour. For the IgG1, HRP-conjugated goat anti-human IgG1 Fc (Sigma-Aldrich, St. Louis, MO, USA) was used for detection.

For the competition ELISA, 13 nM of biotinylated IgG 17 together with 2-fold serial diluted IgG1 3G12 at 667 nM initial concentration were incubated for 1 hour at 37° C., and the bound biotinylated IgG1 17 was detected by streptavidin-HRP antibody (P9170, Sigma-Aldrich). Finally, the reaction was developed by 3,3′,5,5′-tetramethylbenzidine (TMB, Sigma-Aldrich, St. Louis, MO, USA) and was stopped by TMB stop buffer (ScyTek Laboratories, Logan, UT, USA), and the results were recorded with the absorbance at 450 nm. The experiment was performed in duplicate.

### BLItz

The affinity and avidity of the anti-hENPP1 antibody were detected by biolayer interferometry BLItz (ForteBio, Menlo Park, CA, USA). Briefly, DPBS was used to establish a baseline for 30 seconds, and streptavidin biosensor (ForteBio) was coated with 16.7 μg/mL recombinant ENPP1-Biotin for 2 minutes. Different doses of IgG1 and Fab were used for association and monitored for 2 minutes to measure the avidity and affinity. Antigen-coated biosensors with PBS served as a reference control. The dissociation was monitored in DPBS for 4 minutes.

### Cells Lines

Expi 293 cells were purchased from Thermo Fisher Scientific (Waltham, MA, USA) and maintained in Expi 293 expression medium supplemented with 0.4% penicillin-streptomycin (P/S). HepG2 cells and 293T cells were purchased from ATCC and were maintained in EMEM and DMEM medium supplemented with 10% FBS and 1% P/S separately. 293T-ENPP1 cells, stably expressing ENPP1, were prepared by transfecting 293T cells with PEI and selected in complete DMEM with 100 μg/mL Zeocin® (Thermo Fisher Scientific).

### CAR T Cell Construction

For the construction of CAR T cells, anti-human ENPP1 scFv 17 and scFv 3G12 converted from the Fab format were inserted into the pLVX-IRES-CAR plasmid. The CAR components included a fully human CD8a followed by 4-1BB and CD3zeta intracellular domain. To collect the lentivirus supernatant, 293T cells were seeded at 2×10⁶ in a 10 cm plate one day before transfection, the plasmids encoding anti-ENPP1 CAR, pMD.2G envelope plasmid, and psPAX2 package plasmid were co-transfected into 293T cell by PEI. Lentivirus supernatant was collected after 72 hours and filtered through a 0.22 μm membrane. T cells were isolated from healthy donor's PBMC (Zen-Bio, Durham, NC, USA) by using the human Pan T cell Isolation Kit (Miltenyi Biotec, North Rhine-Westphalia, Germany) and stimulated with CD3/CD28 T cell activator Dyna beads (Gibco) in X-VIVO 15 (Lonza, Basel, Switzerland) supplement with 2% human serum (Sigma-Aldrich) and 50 IU/mL IL-2 (Miltenyi Biotec). 48 hours after initiating T cell activation, dyna beads were removed, and the medium was changed to TEXMACS (Miltenyi Biotec) with 50 IU/mL IL-2. T cells were transduced with lentivirus supernatant plus 8 μg/mL polybrene (Sigma-Aldrich) followed by centrifuge for 90 minutes at 800×g, acceleration and deceleration without break, and then incubated at 37° C. After 24 hours, medium with virus was changed, and the T cells were continued in culture in TEXMACS with IL-2 for expansion. Transduction efficiencies were detected by flow cytometry, and CAR T cell function was analyzed 3-4 days later.

### BiTE Production and Antibody Drug Conjugates (ADC) Generation

For the construction of BiTE, humanized OKT3 was inserted at the C-terminal of IgG1 17 and IgG1 3G12 light chain separately with a (G₄S)₃ linker (SEQ ID NO:78). The BiTE proteins were expressed by Expi293 expression system and purified by protein A resin. For the ADC preparation, the IgG1 17 and IgG1 3G12 were diluted in 30% PPG/DPBS buffer separately, and the Osu-Glu-vc-PAB-MMAE (Levena Biopharma, San Diego, CA, USA) cytotoxic drug was diluted in DMSO/PPG (1:1) at a concentration of 10 mM. The IgG1 were treated with MMAE at a drug antibody molar ratio (DAR) of 10:1 and incubated at room temperature for 24 hours with stirring. The buffer was then changed to 0.1% PPG/DPBS by gradient decrease PPG concentration.

### Flow Cytometry

To detect the cells surface expression levels of ENPP1 protein, the cells were stained with anti-ENPP1/PC1 antibody (Abcam, Cambridge, UK) for 30 minutes at 4° C. and then stained with Alexa Fluor 488-conjugated goat anti-rabbit IgG H&L (Abcam) for 30 minutes at 4° C. To determine the cell surface binding of the isolated antibodies, cells were incubated with hlgG1 17, hlgG1 3G12, scFv 17, or scFv 3G12 for 1 hour at 4° C. and then stained with PE-conjugated goat anti-human IgG (Sigma-Aldrich) or APC-conjugated anti-FLAG (Miltenyi Biotec) for 30 minutes at 4° C. For anti-ENPP1-CAR expression on T cells, CD4⁺ T and CD8⁺ T cells were gated by using PerCP conjugated anti-human CD3 antibody (Biolegend, San Diego, CA, USA) and followed by APC conjugated anti-human CD4 antibody (Biolegend) and FITC conjugated anti-human CD8 antibody (Biolegend) separately, and CAR expression was examined by using biotinylated ENPP1 followed by PE Streptavidin (Biolegend).

### Cytotoxicity Assay

The cell-killing activity of anti-ENPP1 CAR⁺ T cells and BiTEs were measured by LDH-Glo cytotoxicity assay kit (Promega, Madison, WI, USA). For the cell-killing activity of anti-ENPP1 CAR⁺ T cells, control T cells or CAR⁺ T cells as effector cells were incubated with 293T, 293T-ENPP1, and HepG2 target cells (5×10³ cells/well in 96-well plate) separately at indicated E:T ratio for 24 hours at 37° C. For the cell-killing activity of BiTEs, target cells (1×10⁴ cells/well) were mixed with serially diluted BiTEs in 50 μL of growth medium, and 50 L activated T cells were added at the ET ratio 10:1 and cultured for 24 hours at 37° C. in 5% CO₂ humidified atmosphere. Controls for the calculation of percentage cytotoxicity were performed according to the manufacturer's instructions. The calculation of relative % cytotoxicity as follows: relative % cytotoxicity=100×(Experimental LDH release-medium background)/(target and effector maximum LDH release control-medium background). For the cell-killing activity of ADC, 293T, 293T-ENPP1, and HepG2 cells were seeded at 2000 cells/well in 96-well white plate and incubated overnight at 37° C. in 5% CO₂. Serial diluted anti-ENPP1 ADC was added and cultured for 4 days at 37° C. in 5% CO₂. Images were captured under a 4×objective microscope (Olympus microscope, Westborough, MA, USA). Controls with cells only were used for calculation of percent relative cell viability. At the end of the time point, the Cell Titer-Glo Luminescent cell viability assay kit (Promega) was used to measure the cell viability. The calculation of % Relative cell viability was as follows: % Relative cell viability=100×(Experimental luminescence values-medium Background)/(Control luminescence values-medium Background). For ADCC, the ADCC reporter bioassay complete kit (Promega) was used, the experiment was performed following the manufacturer's instructions. The 293T-ENPP1 cell line was used as target cell.

### Cytokine Release Assay by ELISA

Control T cells and anti-ENPP1 CAR⁺ T cells were plated the same as the cytotoxicity assays. The supernatant was harvested after 24 hour culturing of cells and detected for cytokine production by using IFNγ and Granzyme B ELISA kit (Thermofisher), following the manufacturer's instructions.

### Results

### Selection of High-Affinity Fab Antibodies Against Human ENPP1

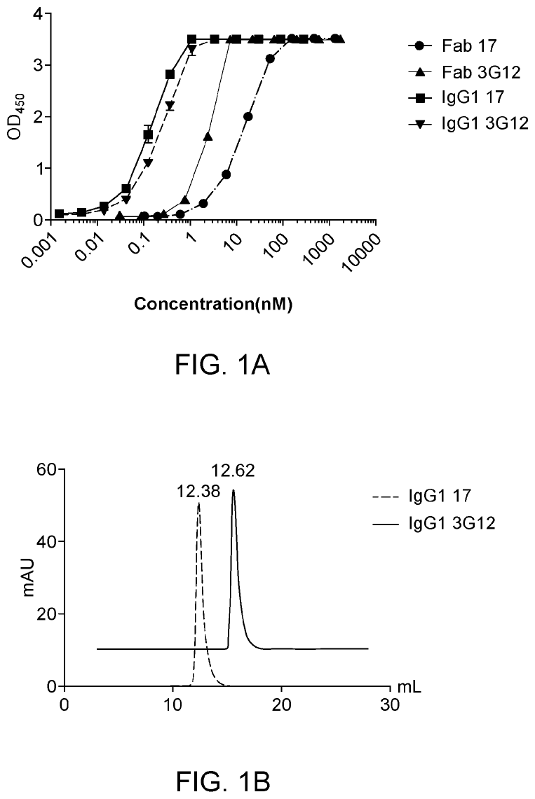

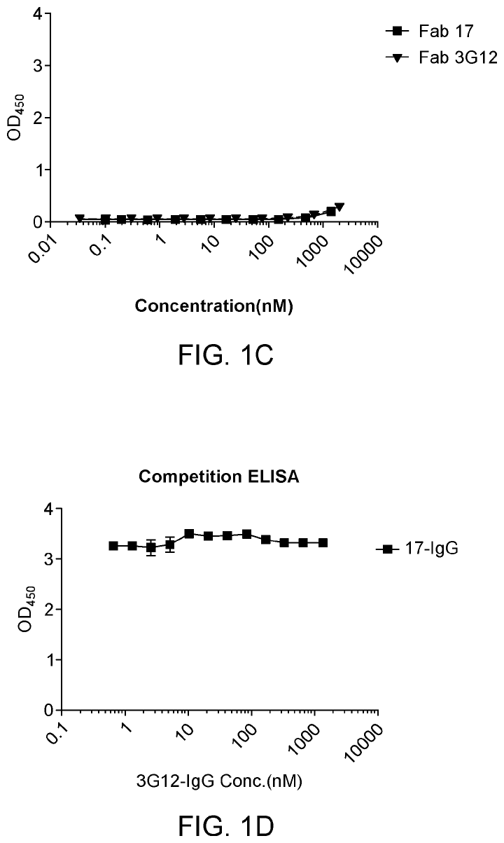

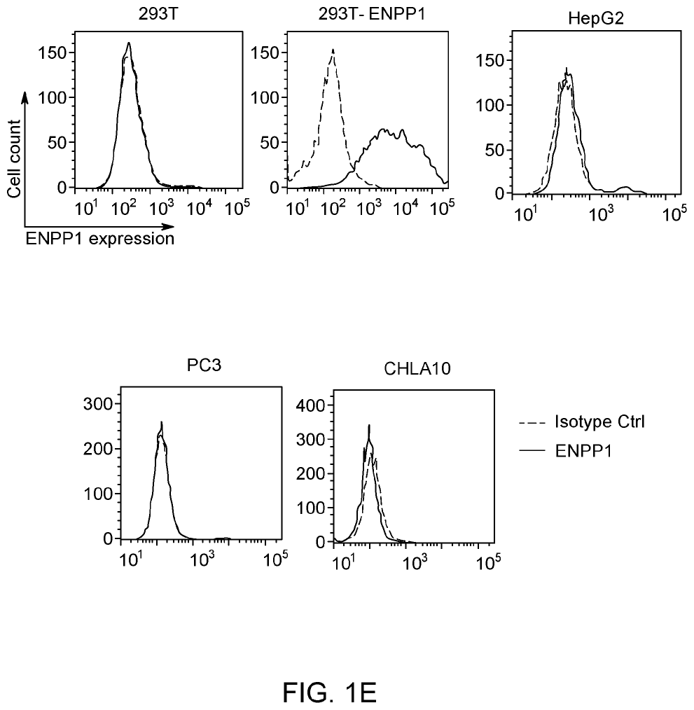

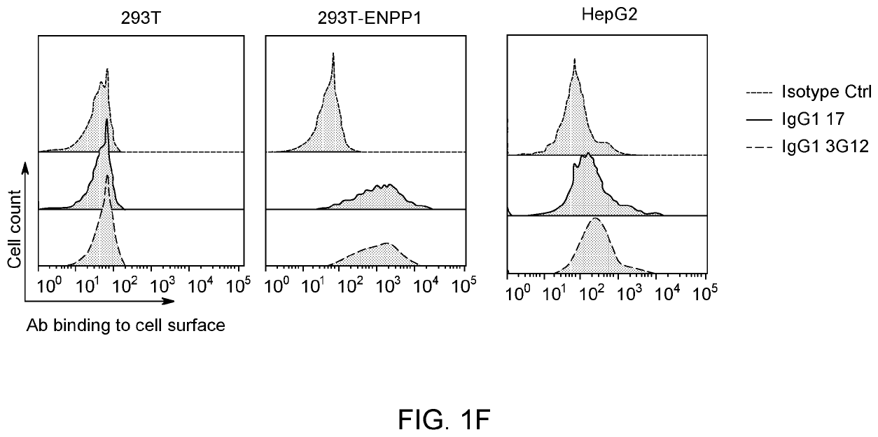

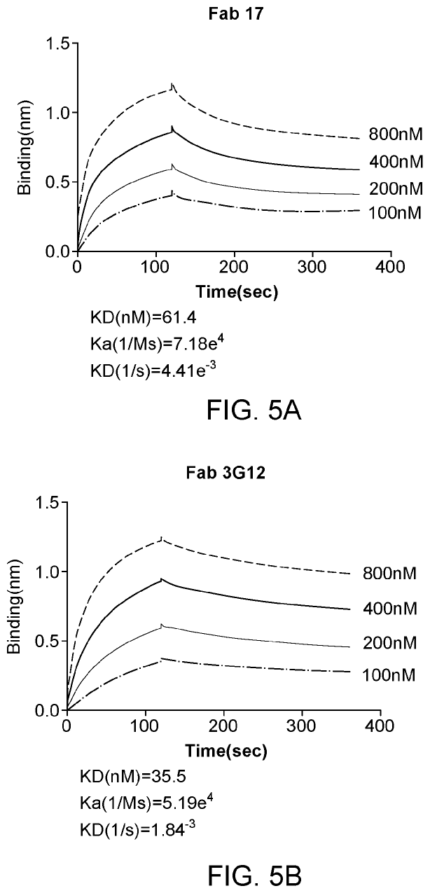

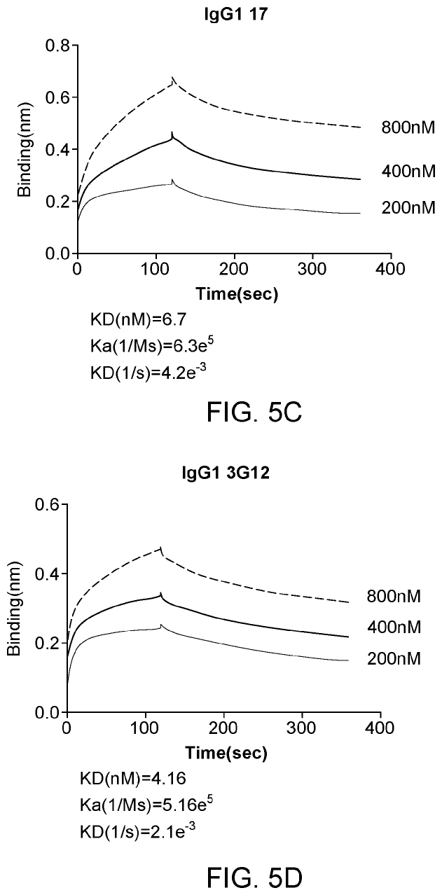

A large phage-displayed human Fab library was used for panning against recombinant human ENPP1 (rhENPP1). After three rounds of panning, several Fab binders were screened. Among these binders, two high-affinity antibodies named Fab 17 and 3G12 were identified based on their high affinity, specificity, and desirable biophysical properties. The EC₅₀ of Fab 17 and 3G12 tested by ELISA were 13.9±0.36 nM and 2.6±0.06 nM, respectively (FIG. 1A). The equilibrium dissociation constant (KD) of these two Fab binders (Fab 17 and 3G12) with rhENPP1 measured by BLItz was 61.4 nM and 35.5 nM, respectively (FIGS. 5A and 5B, Table 15). To further test the specificity of these two Fab binders, the binding effects with BSA was tested by ELISA. The result showed that Fab 17 and 3G12 did not bind to BSA (FIG. 1C).

To increase the avidity and extend the half-life, the two Fab binders were converted to bivalent antibodies by fusion with the human IgG1 Fc. The EC₅₀ of IgG1 17 and IgG1 3G12 tested by ELISA were 0.14±0.01 nM and 0.24±0.01 nM, respectively (FIG. 1A). The equilibrium dissociation constant (KD) of IgG1 17 and IgG1 3G12 measured by BLItz was 6.7 nM and 4.16 nM, respectively (FIGS. 5C and 5D, Table 15). The form of IgG1 17 and IgG1 3G12 was that of a monomer and had no high molecular weight species measured by SEC (FIG. 1B), and the location of the peak was consistent with the standard protein. As IgG1 17 and IgG1 3G12 have the same heavy chain but different light chains, the competition ELISA was used to test if the two binders bind to the same epitope. As shown in FIG. 1D, IgG1 17 and IgG1 3G12 did not compete with each other, which indicated that the two antibodies binding to the different epitopes of ENPP1.

TABLE 15
Blitz results of anti-human ENPP1 antibodies.

| Antibody | Kₒₙ (M⁻¹s⁻¹)¹ | k_{off} (s⁻¹)¹ | KD (nM)¹ |
|---|---|---|---|
| Fab 17 | 7.2 × 10⁴ | 4.4 × 10⁻³ | 61.4 |
| Fab 3G12 | 5.2 × 10⁴ | 1.8 × 10⁻³ | 35.5 |
| IgG1 17 | 6.3 × 10⁵ | 4.2 × 10⁻³ | 6.7 |
| IgG1 3G12 | 5.2 × 10⁵ | 2.1 × 10⁻³ | 4.2 |

¹Mean kinetic rate constants (kₒₙ, k_{off}) and equilibrium dissociation constants (KD = k_{off}/kₒₙ) were determined from curve fitting analyses of BLItz results.

hlgG1 17 and hlgG1 3G12 have High Binding Effects to ENPP1-Expressing Cells but have No Effects on Inducing Antibody-Dependent Cellular Cytotoxicity (ADCC)

To access the antibody-dependent cellular cytotoxicity (ADCC) of IgG1 17 and IgG1 3G12, the surface expression of ENPP1 as first tested on 293T cells, 293T over-expression ENPP1 293T cells, and three different cancer cell lines (HepG2, PC3, and CHLA10). Among these cell lines, only 293T-ENPP1 cells showed high expression of ENPP1, and HepG2 cancer cells showed a 4.8% low expression level (FIG. 1E). Next, the binding effects of IgG1 17 and IgG1 3G12 to the ENPP1 positive cell 293T-ENPP1 and HepG2 together with the ENPP1 negative cell 293T cell line were analyzed. IgG1 17 and IgG1 3G12 showed specific binding with 293T-ENPP1 cells and demonstrated no binding effects with ENPP1 negative 293T cells (FIG. 1F). Moreover, IgG1 17 and IgG1 3G12 showed higher binding effects with HepG2 cells (11% and 12%, respectively), indicating that IgG1 17 and IgG1 3G12 have high avidity compared with a positive control anti-ENPP1 antibody (Abcam cat #ab240653) (FIG. 1F).

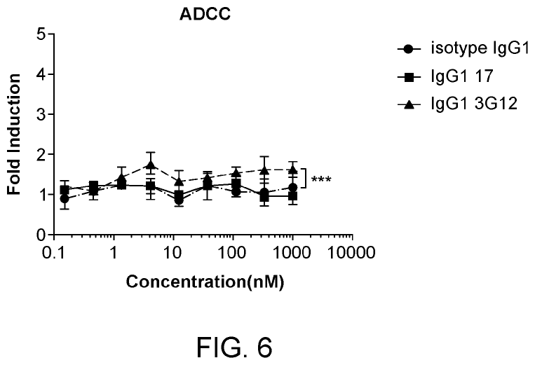

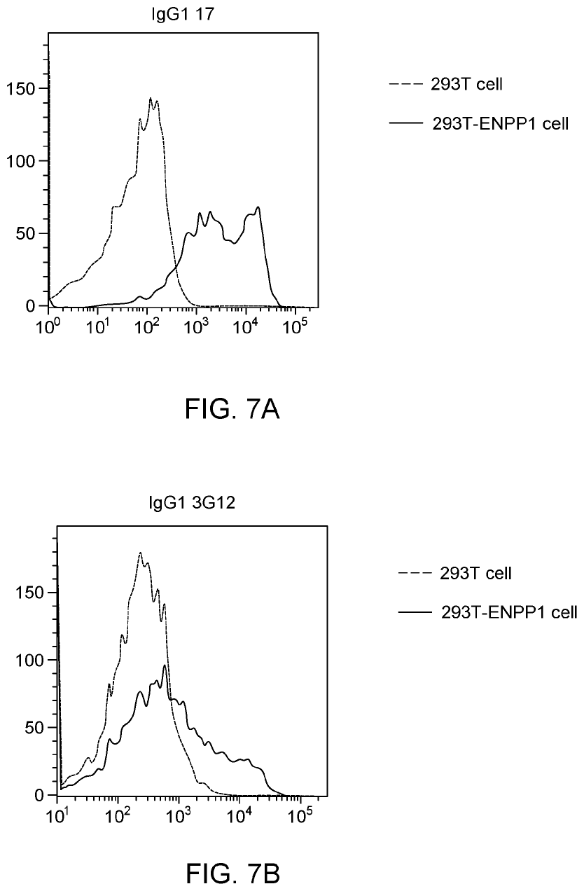

The ADCC activity of IgG1 17 and IgG1 3G12 was next investigated on ENPP1-expressing cells using the ADCC reporter bioassay. The ADCC bioassay effector cells were co-cultured with target cells 293T-ENPP1 in the presence of serial diluted IgG1 17 and IgG1 3G12. The results showed that both IgG1 17 and IgG1 3G12 have no effects on inducing ADCC (FIG. 6). Even though IgG1 3G12 showed a higher effect compared with the control group, it did not have a dose-dependent manner.

Taken together, these data demonstrated that these anti-ENPP1 antibodies could be further developed into effective ADCs and cell engagers (e.g., BiTEs) as IgG1 17 and IgG1 3G12 have high specificity against ENPP1.

### Anti-ENPP1 IgG1-MMAE ADC is Cytotoxic to ENPP1-Expressing Cells

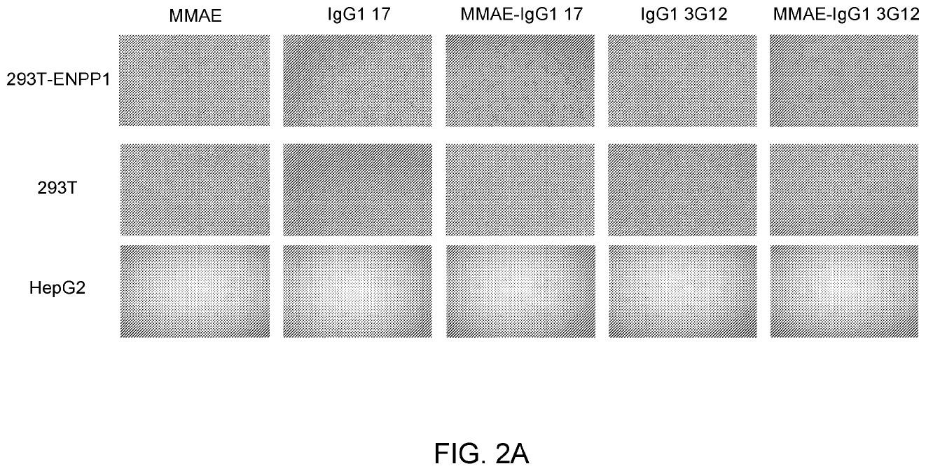

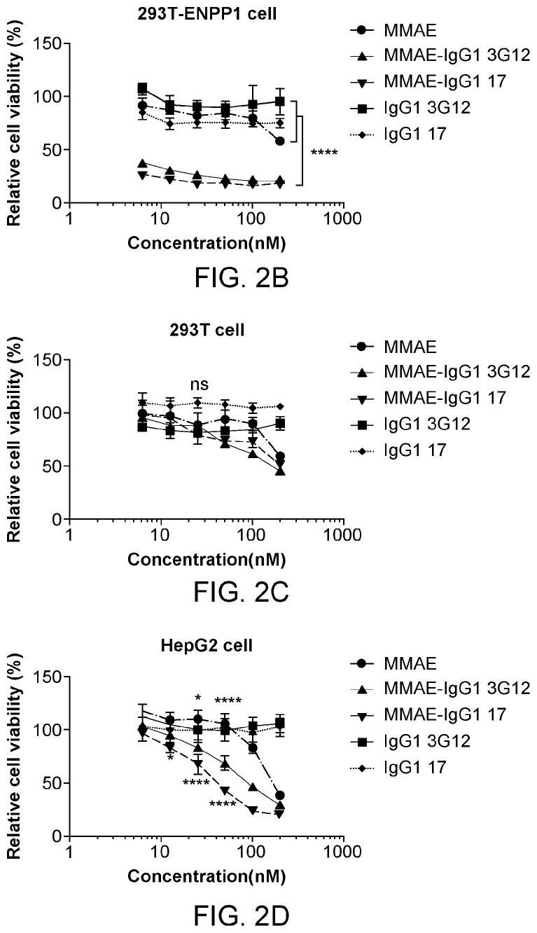

To access the cell cytotoxicity of anti-ENPP1 IgG1-MMAE ADC, the IgG1 internalization between 293T cells and 293T-ENPP1 cells was investigated by flow cytometry. The IgG1 17 and IgG1 3G12 can be efficiently and highly internalized into 293T-ENPP1 cells. The ENPP1-expressing cells 293T-ENPP1 and HepG2 cells were then treated with different concentrations of MMAE-IgG1 17 and MMAE-IgG1 3G12 and free MMAE and IgG1, and the non-specific killing effects with 293T cells, which did not express ENPP1, were tested. MMAE-IgG1 17 and MMAE-IgG1 3G12 demonstrated specific cell killing effects with 293T-ENPP1 (FIGS. 2A and 2B). The estimated IC₅₀ values of MMAE-IgG1 17 and MMAE-IgG1 3G12 ADCs were 13 nM and 10 nM, respectively. The MMAE-IgG1 ADC and free MMAE showed a non-specific killing at the concentration of 200 nM on both 293T and 293T-ENPP1 cells, but there was no non-specific killing at lower concentrations (FIGS. 2A and 2C). Next, the ADC killing effects with HepG2 cancer cells, which have a lower expression level of ENPP1, were tested. The estimated IC₅₀ values of MMAE-IgG1 17 and MMAE-IgG1 3G12 were 33 nM and 80 nM, respectively (FIGS. 2A and 2D). When compared with the 293T cell line, the HepG2 was more sensitive to MMAE. The killing effects of both free MMAE and ADC (MMAE-IgG1 17 and MMAE-IgG1 3G12) were comparable at 200 nM in HepG2 cells.

### Anti-ENPP1 BiTE Showed Potent Cytotoxicity Against ENPP1-Expressing Cells

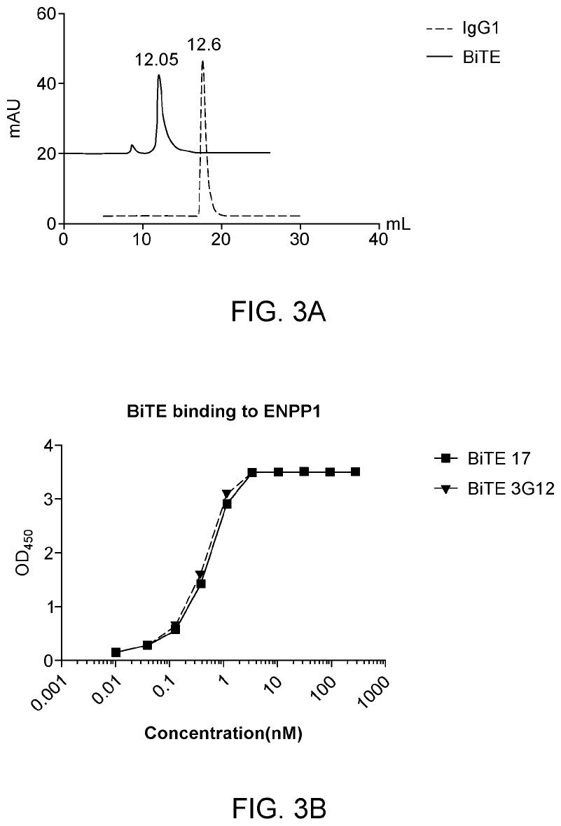

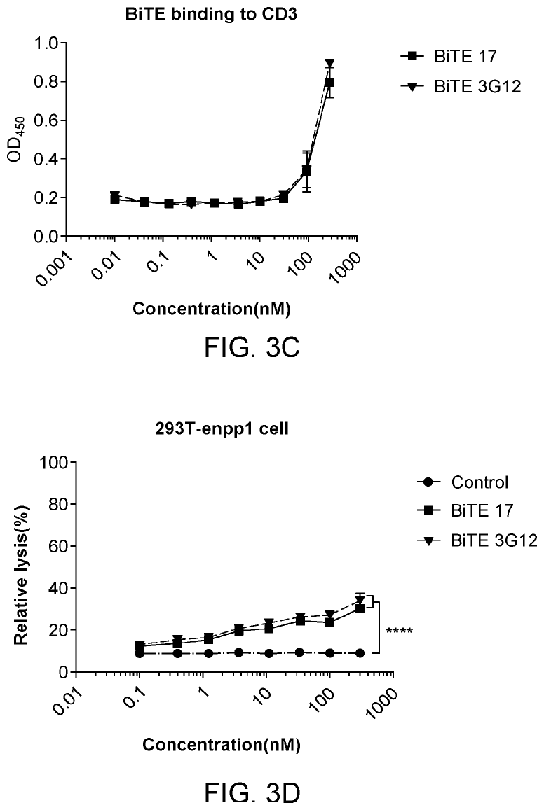

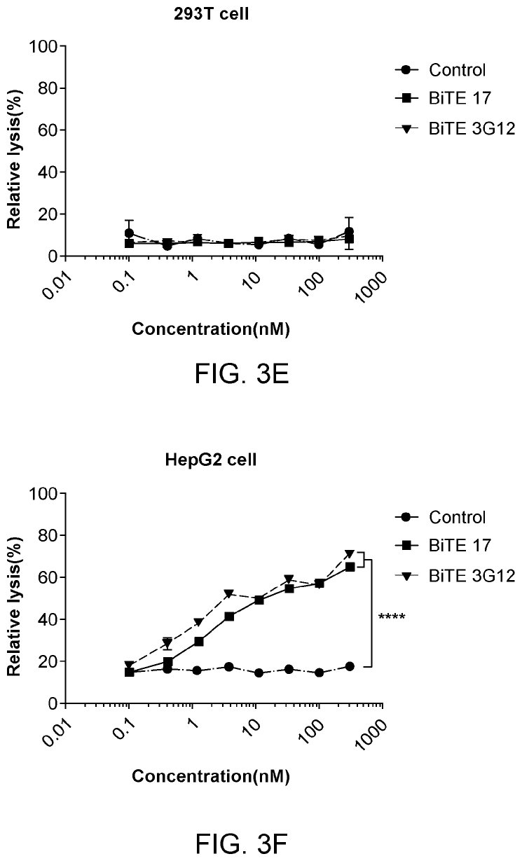

To access the cell cytotoxicity of anti-ENPP1 BiTEs, the IgG1 format BiTE was constructed by adding the humanized OKT3 at the C-terminal of the IgG1 17 and IgG1 3G12 light chains. The protein was characterized by SEC, which confirmed the BiTEs were monomers and had a bigger molecular weight (˜ 200 kDa) than IgG1 (˜ 150 kDa) (FIG. 3A). The EC₅₀ values of BiTE 17 and BiTE 3G12 with human ENPP1 tested by ELISA were 0.5±0.01 nM and 0.4±0.01 nM, respectively (FIG. 3B). The estimated EC₅₀ values of BITE 17 and BiTE 3G12 with CD3 were about 200 nM (FIG. 3C). Next, the T-cell mediated cytotoxicity induced by BiTE was assessed. Dose-dependent lysis induced by BiTE 17 and BiTE 3G12 was observed when targeting 293T-ENPP1 cells at the E:T ratio of 10:1 (FIG. 3D). The killing effects of BiTE 17 and BiTE 3G12 was not observed when targeting ENPP1-negative 293T cells (FIG. 3E), which indicated that the BiTEs were specific for targeting ENPP1. Similar dose-dependent lysis was also found in HepG2 cancer cell lines, and the estimated IC₅₀ values of BiTE 17 and BiTE 3G12 were 3.2±0.5 nM and 1.5±0.6 nM, respectively (FIG. 3F).

### 2^{nd} Generation Anti-ENPP1 CAR T Mediated Cytotoxicity to ENPP1-Expressing Cells

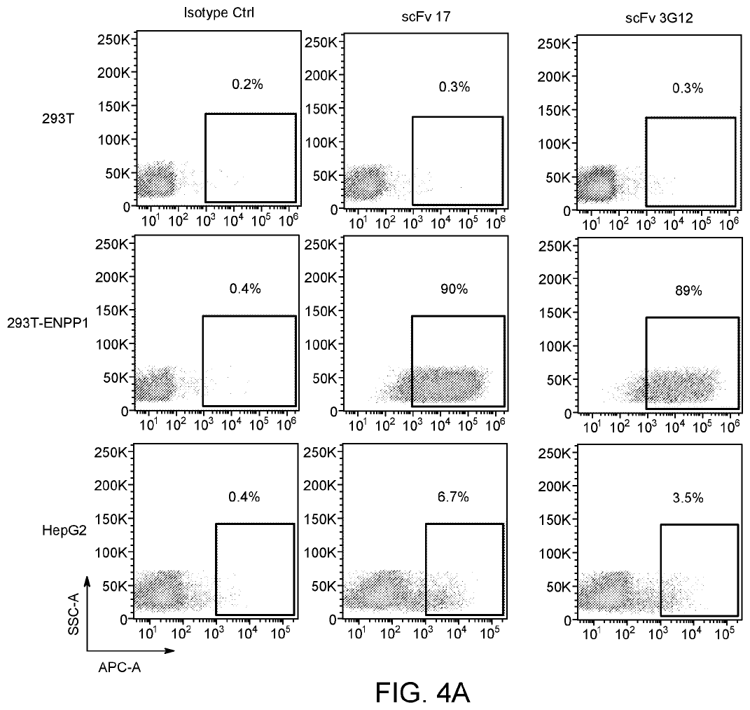

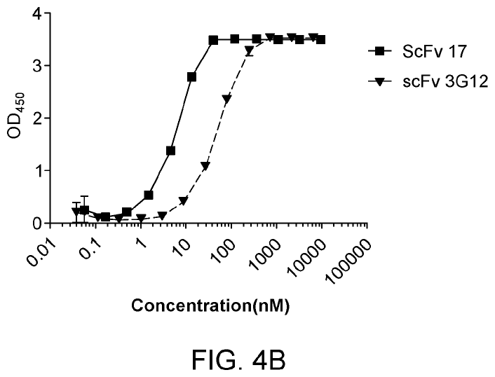

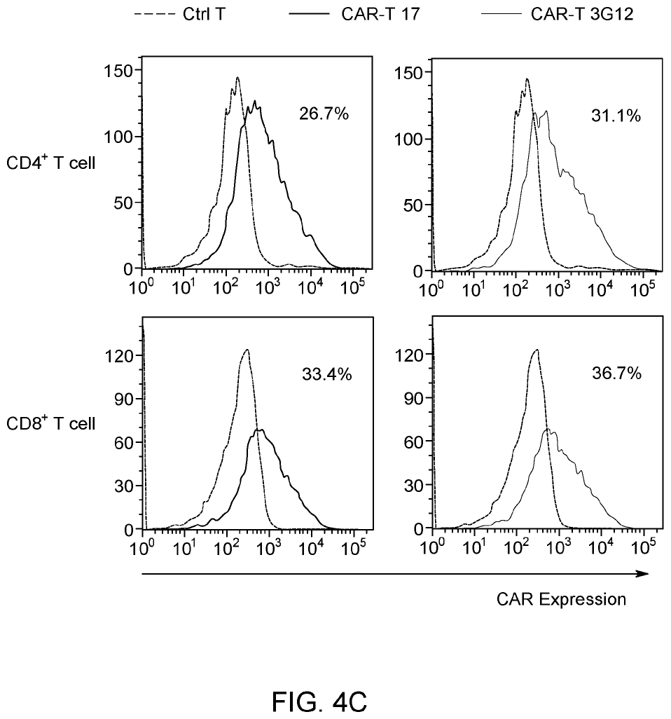

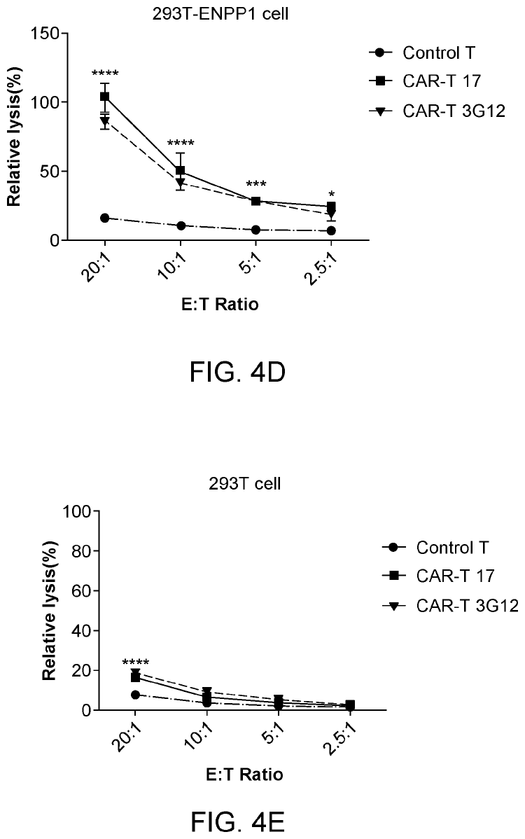

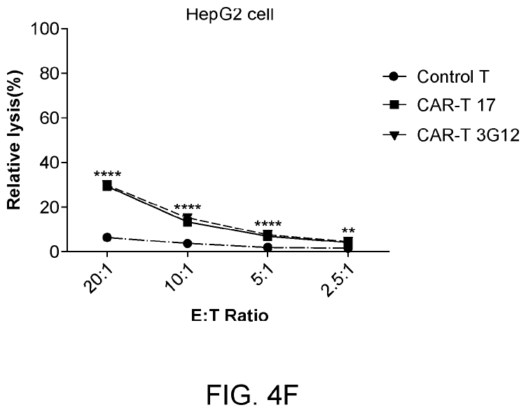

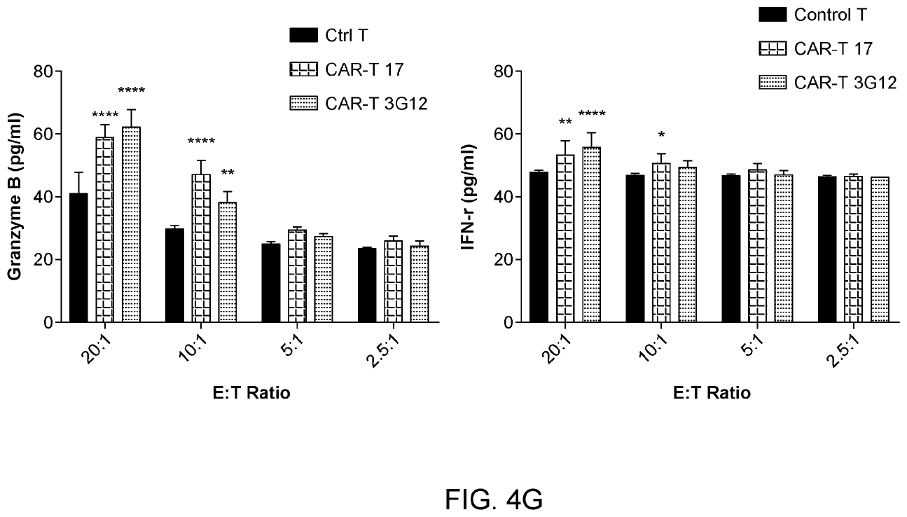

To assess the cytotoxicity of anti-ENPP1 antibodies being used for CAR T cell therapy, lentiviruses that encoded 2^{nd} generation anti-ENPP1 CARs containing the co-stimulatory intracellular domains, a transmembrane domain, and a single chain variable fragment (scFv) derived from either IgG1 17 or 3G12 were generated. To confirm the scFv format still maintained the binding effects with ENPP1, the cell binding ability and affinity of scFv 17 and scFv 3G12 were investigated. The scFvs still specifically bound to 293t-ENPP1 cells and HepG2 cells, and had no binding effect with 293T (FIG. 4A). The EC₅₀ values of scFv 17 and scFv 3G12 tested by ELISA were 6.4±0.25 nM and 52.2±1.83 nM, respectively (FIG. 4B).

Next, T cells were transduced with the 2^{nd} generation CARs, and CAR expression was confirmed by flow cytometry. As shown in FIG. 4C, the expression level of CAR 17 in transduced CD4⁺ T cells was 26.7% and 31.1% in CD8⁺ T cells. The expression level of CAR 3G12 in transduced CD4⁺ T cells were 33.4% and 36.7% in CD8⁺ T cells. To determine the cytotoxicity of anti-ENPP1 CAR T cells in vitro, CAR 17-expressing T cells or CAR 3G12-expressing T cells were co-cultured with ENPP1-positive cells (293T-ENPP1 or HepG2 cells) at the E:T ratios of 20:1, 10:1, 5:1, and 2.5:1 for 24 hours. Both CAR T cells showed higher lysis than the control group by targeting 293T-ENPP1 and HepG2 cells (FIGS. 4D and F). The CAR T cells showed lower lysis by targeting HepG2 than 293T-ENPP1; this was mainly due to the low expression level of ENPP1 by HepG2 cells. To test the specificity of the CAR T cells, the ENPP1-negative 293T cells were used as target cells at the same E:T ratios. The result showed that there was little non-specific killing at the high E:T ratio, but the percent lysis was very low (FIG. 4E). To further analyze the in vitro mechanism of CAR T cell mediated cytotoxicity, the Granzyme B and IFN-γ secretion levels were assessed after 24-hour co-incubation of CAR T cells and 293T-ENPP1 cells at different E:T ratios. The secretion level of granzyme B was significantly elevated, and the level of IFN-γ was slightly elevated at the E:T ratio of 20:1 and 10:1 compared with the control group (FIG. 4G).

Together, these results demonstrate that binders having two sets of three CDRs described herein (e.g., SEQ ID NOs: 1-3 and 9-11 or SEQ ID NOs: 17-19 and 25-27) can bind to an ENPP1 polypeptide. Also as demonstrated herein, these binders can be engineered into molecules (e.g., antibodies, antigen binding fragments, antibody domains, CARs, cell engagers (e.g., BiTEs), or ADCs (e.g., antibody-radioconjugates)) that can be used to a treat a mammal (e.g., a human) having cancer (e.g., a sarcoma such as Ewing sarcoma).

### Example 2: Exemplary ENPP1 Polypeptides

This Example provides an amino acid sequence of a human ENPP1 polypeptide (SEQ ID NO: 150). The underlined and bolded amino acid sequence of this human ENPP1 polypeptide depicts the ENPP1 extracellular domain (SEQ ID NO:151) and was used to identify ENPP1 binders.

(SEQ ID NO: 150)
MERDGCAGGGSRGGEGGRAPREGPAGNGRDRGRSHAAEAPGDPQA
AASLLAPMDVGEEPLEKAARARTAKDPNTYKVLSLVLSVCVLTTI
LGCIFGLKPSCAKEVKSCKGRCFERTFGNCRCDAACVELGNCCLD
YQETCIEPEHIWTCNKFRCGEKRLTRSLCACSDDCKDKGDCCINY
SSVCQGEKSWVEEPCESINEPQCPAGFETPPTLLFSLDGFRAEYL
HTWGGLLPVISKLKKCGTYTKNMRPVYPTKTFPNHYSIVTGLYPE
SHGIIDNKMYDPKMNASFSLKSKEKFNPEWYKGEPIWVTAKYQGL
KSGTFFWPGSDVEINGIFPDIYKMYNGSVPFEERILAVLQWLQLP
KDERPHFYTLYLEEPDSSGHSYGPVSSEVIKALQRVDGMVGMLMD
GLKELNLHRCLNLILISDHGMEQGSCKKYIYLNKYLGDVKNIKVI
YGPAARLRPSDVPDKYYSFNYEGIARNLSCREPNQHFKPYLKHFL
PKRLHFAKSDRIEPLTFYLDPQWQLALNPSERKYCGSGFHGSDNV
FSNMQALFVGYGPGFKHGIEADTFENIEVYNLMCDLLNLTPAPNN
GTHGSLNHLLKNPVYTPKHPKEVHPLVQCPFTRNPRDNLGCSCNP
SILPIEDFQTQFNLTVAEEKIIKHETLPYGRPRVLQKENTICLLS
QHQFMSGYSQDILMPLWTSYTVDRNDSFSTEDFSNCLYQDFRIPL
SPVHKCSFYKNNTKVSYGFLSPPQLNKNSSGIYSEALLTTNIVPM
YQSFQVIWRYFHDTLLRKYAEERNGVNVVSGPVFDFDYDGRCDSL
ENLRQKRRVIRNQEILIPTHFFIVLTSCKDTSQTPLHCENLDTLA
FILPHRTDNSESCVHGKHDSSWVEELLMLHRARITDVEHITGLSF
YQQRKEPVSDILKLKTHLPTFSQED

### Example 3: Exemplary Fabs Having the Ability to Bind an ENPP1 Polypeptide

This Example provides the amino acid sequences of the heavy chain variable domain and the light chain variable domain of a Fab designated Clone #1 (also referred to as 17-Fab). The CDRs, framework sequences, and constant domains of each also are provided and delineated.

Anti-ENPP1 Clone #1 (17-Fab) heavy chain
Heavy chain variable domain
(without first constant domain and partial hinge):
(SEQ ID NO: 8)
QVQLVESGGGLVKPGGSLRLSCAASGFTFSDYYMSWIRQAPGKGLEWVSYISSSGS
TIYYADSVKGRFTISRDNAKNSLYLQMNSLRAEDTAVYYCAREVYYYDSSGYYTY
YFDYWGQGTLVTVSS
Framework Region 1 of heavy chain variable domain:
(SEQ ID NO: 4)
QVQLVESGGGLVKPGGSLRLSCAAS
CDR1 of heavy chain variable domain:
(SEQ ID NO: 1)
GFTFSDYY
Framework Region 2 of heavy chain variable domain:
(SEQ ID NO: 5)
MSWIRQAPGKGLEWVSY
CDR2 of heavy chain variable domain:
(SEQ ID NO: 2)
ISSSGSTI
Framework Region 3 of heavy chain variable domain:
(SEQ ID NO: 6)
YYADSVKGRFTISRDNAKNSLYLQMNSLRAEDTAVYYC
CDR3 of heavy chain variable domain:
(SEQ ID NO: 3)
AREVYYYDSSGYYTYYFDY
Framework Region 4 of heavy chain variable domain:
(SEQ ID NO: 7)
WGQGTLVTVSS
Human IgG1 heavy chain constant domain 1 (CH1):
(SEQ ID NO: 33)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKV
Human immunoglobulin G1 hinge region (partial):
(SEQ ID NO: 152)
EPKSCDKT
Anti-ENPP1 Clone #1 (17-Fab) light chain
Light chain variable domain (without kappa/lambda constant domain):
(SEQ ID NO: 16)
DIQMTQSPSSLSASVGDRVTITCRASQSISSYLNWYQQKPGKAPKLLIYAASSLQSGV
PSRFSGSGSGTDFTLTISSLQPEDAATYYCQQYDTFPLTFGGGTKVEIKR
Framework Region 1 of light chain variable domain:
(SEQ ID NO: 12)
DIQMTQSPSSLSASVGDRVTITCRAS
CDR1 of light chain variable domain:
(SEQ ID NO: 9)
QSISSY
Framework Region 2 of light chain variable domain:
(SEQ ID NO: 13)
LNWYQQKPGKAPKLLIY
CDR2 of light chain variable domain:
(SEQ ID NO: 10)
AASSLQS
Framework Region 3 of light chain variable domain:
(SEQ ID NO: 14)
GVPSRFSGSGSGTDFTLTISSLQPEDAATYYC
CDR3 of light chain variable domain:
(SEQ ID NO: 11)
QQYDTFPLT
Framework Region 4 of light chain variable domain:
(SEQ ID NO: 15)
FGGGTKVEIKR
Human kappa light chain constant domain:
(SEQ ID NO: 34)
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

### Example 4: Exemplary Fabs Having the Ability to Bind an ENPP1 Polypeptide

This Example provides the amino acid sequences of the heavy chain variable domain and the light chain variable domain of a Fab designated Clone #2 (also referred to as 3G12-Fab). The CDRs, framework sequences, and constant domains of each also are provided and delineated.

Anti-ENPP1 Clone #2 (3G12-Fab)
Heavy chain variable domain
(without first constant domain and partial hinge):
(SEQ ID NO: 24)
QVQLVESGGGLVKPGGSLRLSCAASGFTFSDYYMSWIRQAPGKGLEWVSYISSSGS
TIYYADSVKGRFTISRDNAKNSLYLQMNSLRAEDTAVYYCAREVYYYDSSGYYTY
YFDYWGQGTLVTVSS
Framework Region 1 of heavy chain variable domain:
(SEQ ID NO: 20)
QVQLVESGGGLVKPGGSLRLSCAAS
CDR1 of heavy chain variable domain:
(SEQ ID NO: 17)
GFTFSDYY
Framework Region 2 of heavy chain variable domain:
(SEQ ID NO: 21)
MSWIRQAPGKGLEWVSY
CDR2 of Heavy chain variable domain:
(SEQ ID NO: 18)
ISSSGSTI
Framework Region 3 of heavy chain variable domain:
(SEQ ID NO: 22)
YYADSVKGRFTISRDNAKNSLYLQMNSLRAEDTAVYYC
CDR3 of heavy chain variable domain:
(SEQ ID NO: 19)
AREVYYYDSSGYYTYYFDY
Framework Region 4 of heavy chain variable domain:
(SEQ ID NO: 23)
WGQGTLVTVSS
Human IgG1 heavy chain constant domain 1 (CH1):
(SEQ ID NO: 33)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKV
Human immunoglobulin Gl hinge region (partial):
(SEQ ID NO: 152)
EPKSCDKT
Anti-ENPP1 Clone #2 (3G12-Fab)
Light chain variable domain (without kappa/lambda constant domain):
(SEQ ID NO: 32)
DIQMTQSPSSLSASVGDRVTITCRASQSISSYLNWYQQKPGKAPKLLIYAASSLQSGV
PSRFSGSGSGTDFTLTISSLQPEDVAVYYCQQYYTSRITFGQGTKLEIKR
Framework Region 1 of light chain variable domain:
(SEQ ID NO: 28)
DIQMTQSPSSLSASVGDRVTITCRAS
CDR1 of light chain variable domain:
(SEQ ID NO: 25)
QSISSY
Framework Region 2 of light chain variable domain:
(SEQ ID NO: 29)
LNWYQQKPGKAPKLLIY
CDR2 of light chain variable domain:
(SEQ ID NO: 26)
AASSLQS
Framework Region 3 of light chain variable domain:
(SEQ ID NO: 30)
GVPSRFSGSGSGTDFTLTISSLQPEDVAVYYC
CDR3 of light chain variable domain:
(SEQ ID NO: 27)
QQYYTSRIT
Framework Region 4 of light chain variable domain:
(SEQ ID NO: 31)
FGQGTKLEIKR
Human Kappa light chain constant domain:
(SEQ ID NO: 34)
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

### Example 5: Nucleic Acids Encoding Exemplary ENPP1 Binding Molecules

This Example provides the nucleic acid sequences encoding the indicated chains/domains of Clones #1-#2.

Clone #1
Nucleic acid encoding SEQ ID NO: 8
(Clone #1; Heavy Chain):
(SEQ ID NO: 35)
CAAGTTCAATTAGTGGAGAGCGGCGGCGGATTGGTTAAACCGGGG
GGCAGTCTGCGTTTAAGCTGCGCAGCTTCCGGGTTTACTTTTTCG
GATTACTATATGAGCTGGATTCGTCAGGCTCCCGGTAAAGGGTTG
GAATGGGTTAGCTACATCTCAAGTTCCGGCTCTACAATCTACTAT
GCAGATTCCGTGAAAGGGCGCTTCACCATCTCTCGTGACAATGCA
AAGAACAGTTTATACTTACAAATGAACTCGCTGCGTGCGGAAGAT
ACGGCCGTGTATTACTGTGCGAGAGAGGTATATTACTATGATAGT
AGTGGTTATTATACCTACTACTTTGACTACTGGGGCCAGGGAACC
CTGGTCACTGTCTCCTCA
Nucleic acid encoding SEQ ID NO: 16
(Clone #1; Light Chain):
(SEQ ID NO: 36)
GATATTCAAATGACCCAGTCTCCTTCTTCCTTGAGCGCGTCTGTG
GGAGACCGTGTAACTATCACGTGTCGTGCATCCCAAAGCATTTCG
AGTTACTTGAACTGGTATCAACAAAAGCCTGGTAAAGCGCCCAAA
CTGTTGATCTATGCTGCCAGTAGTCTTCAATCTGGTGTGCCCTCG
CGCTTCTCAGGTTCGGGGTCAGGAACGGACTTTACCCTGACCATT
AGCTCTCTGCAACCGGAAGATGCTGCAACGTATTACTGTCAACAA
TATGATACTTTTCCACTCACTTTCGGCGGAGGGACCAAAGTGGAG
ATCAAA
Clone #2
Nucleic acid encoding SEQ ID NO: 24
(Clone #2; Heavy Chain):
(SEQ ID NO: 37)
CAAGTTCAATTAGTGGAGAGCGGCGGCGGATTGGTTAAACCGGGG
GGCAGTCTGCGTTTAAGCTGCGCAGCTTCCGGGTTTACTTTTTCG
GATTACTATATGAGCTGGATTCGTCAGGCTCCCGGTAAAGGGTTG
GAATGGGTTAGCTACATCTCAAGTTCCGGCTCTACAATCTACTAT
GCAGATTCCGTGAAAGGGCGCTTCACCATCTCTCGTGACAATGCA
AAGAACAGTTTATACTTACAAATGAACTCGCTGCGTGCGGAAGAT
ACGGCCGTGTATTACTGTGCGAGAGAGGTATATTACTATGATAGT
AGTGGTTATTATACCTACTACTTTGACTACTGGGGCCAGGGAACC
CTGGTCACTGTCTCCTCA
Nucleic acid encoding SEQ ID NO: 32
(Clone #2; Light Chain):
(SEQ ID NO: 38)
GATATTCAAATGACCCAGTCTCCTTCTTCCTTGAGCGCGTCTGTG
GGAGACCGTGTAACTATCACGTGTCGTGCATCCCAAAGCATTTCG
AGTTACTTGAACTGGTATCAACAAAAGCCTGGTAAAGCGCCCAAA
CTGTTGATCTATGCTGCCAGTAGTCTTCAATCTGGTGTGCCCTCG
CGCTTCTCAGGTTCGGGGTCAGGAACGGACTTTACCCTGACCATT
AGCTCTCTGCAACCGGAAGATGTGGCAGTTTATTACTGTCAACAA
TATTACACTTCTCGTATCACGTTCGGCCAAGGGACCAAGCTGGAA
ATCAAA

### Example 6: Exemplary IgGs Having the Ability to Bind an ENPP1 Polypeptide

This Example provides the structure of an exemplary Ig and provides the amino acid and nucleic acid sequences of an exemplary hinge, CH2, and CH3 regions/domains.

Exemplary Ig (e.g., IgG1) structure
Heavy chain: Heavy Chain Variable Domain +
CH1 + Hinge + CH2 + CH3
Light chain: Light Chain Variable Domain +
Constant Light (Kappa or Lambda)
Human immunoglobulin G1 hinge region:
(SEQ ID NO: 39)
EPKSCDKTHTCPPCP
(SEQ ID NO: 40)
GAGCCCAAATCTTGTGACAAAACTCACACATGCCCACCGTGCCCA
Human immunoglobulin G1 heavy chain constant
domains 2 and 3 (CH2-CH3):
(SEQ ID NO: 41)
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSRDELTKNQVSL
TCLVKGFYPSDIAVEWESNGQPENNYKTTPPVLDSDGSFFLYSKL
TVDKSRWQQGNVFSCSVMHEALHNHYTQKSLSLSPGK
(SEQ ID NO: 42)
GCACCTGAACTCCTGGGGGGACCGTCAGTCTTCCTCTTCCCCCCA
AAACCCAAGGACACCCTCATGATCTCCCGGACCCCTGAGGTCACA
TGCGTGGTGGTGGACGTGAGCCACGAAGACCCTGAGGTCAAGTTC
AACTGGTACGTGGACGGCGTGGAGGTGCATAATGCCAAGACAAAG
CCGCGGGAGGAGCAGTACAACAGCACGTACCGTGTGGTCAGCGTC
CTCACCGTCCTGCACCAGGACTGGCTGAATGGCAAGGAGTACAAG
TGCAAGGTCTCCAACAAAGCCCTCCCAGCCCCCATCGAGAAAACC
ATCTCCAAAGCCAAAGGGCAGCCCCGAGAACCACAGGTGTACACC
CTGCCCCCATCCCGGGATGAGCTGACCAAGAACCAGGTCAGCCTG
ACCTGCCTGGTCAAAGGCTTCTATCCCAGCGACATCGCCGTGGAG
TGGGAGAGCAATGGGCAGCCGGAGAACAACTACAAGACCACGCCT
CCCGTGCTGGACTCCGACGGCTCCTTCTTCCTCTACAGCAAGCTC
ACCGTGGACAAGAGCAGGTGGCAGCAGGGGAACGTCTTCTCATGC
TCCGTGATGCATGAGGCTCTGCACAACCACTACACGCAGAAGAGC
CTCTCCCTGTCTCCGGGTAAA

### Example 7: Exemplary scFvs Having the Ability to Bind an ENPP1 Polypeptide

This Example provides structures of exemplary scFv's, as well as the amino acid sequences of an exemplary heavy chain variable domain and an exemplary light chain variable domain of an exemplary scFv. The CDRs and framework sequences of each also are delineated. An exemplary linker amino acid sequence such as a linker amino acid sequence set forth in Example 11 can be used to link the heavy chain variable domain and the light chain variable domain together to form a scFv. Structures of exemplary scFv's including the amino acid and nucleic acid sequences with the linker, CDRs, and framework sequences delineated are shown.

Exemplary scFv structure:
Heavy Chain Variable Domain/Region + Linker + Light
Chain Variable Domain/Region
Exemplary scFv structure:
Light Chain Variable Domain/Region + Linker + Heavy
Chain Variable Domain/Region
Exemplary scFv heavy chain variable domain
Variable Heavy Region:
QVQLVESGGGLVKPGGSLRLSCAAS(X1)ₙMSWIRQAPGKGLEWVS
Y(X2)ₙYYADSVKGRFTISRDNAKNSLYLQMNSLRAEDTAVYYC
(X3)ₙWGQGTLVTVSS
(SEQ ID NO: 43 which includes SEQ ID NO: 4
followed by a CDR1 of a variable heavy region,
followed by SEQ ID NO: 5, followed by a CDR2
of a variable heavy region, followed by
SEQ ID NO: 6, followed by a CDR3 of a variable
heavy region, followed by SEQ ID NO: 7)
Framework Region 1 of Variable Heavy Region:
(SEQ ID NO: 4)
QVQLVESGGGLVKPGGSLRLSCAAS
CDR1 of Variable Heavy Region:
(X1)ₙ
Framework Region 2 of Variable Heavy Region:
(SEQ ID NO: 5)
MSWIRQAPGKGLEWVSY
CDR2 of Variable Heavy Region:
(X2)ₙ
Framework Region 3 of Variable Heavy Region:
(SEQ ID NO: 6)
YYADSVKGRFTISRDNAKNSLYLQMNSLRAEDTAVYYC
CDR3 of Variable Heavy Region:
(X3)ₙ
Framework Region 4 of Variable Heavy Region:
(SEQ ID NO: 7)
WGQGTLVTVSS
Exemplary scFv light chain variable domain
Variable Light Region:
DIQMTQSPSSLSASVGDRVTITCRAS(X4)ₙLNWYQQKPGKAPKLL
IY(X5)ₙGVPSRFSGSGSGTDFTLTISSLQPEDAATYYC(X6)ₙF
GGGTKVEIKR
(SEQ ID NO: 44 which includes SEQ ID NO: 12,
followed by a CDR1 of a variable light region,
followed by SEQ ID NO: 13, followed by a CDR2
of a variable light region, followed by SEQ
ID NO: 14, followed by a CDR3 of a variable
light region, followed by SEQ ID NO: 15)
Framework Region 1 of Variable Light Region:
(SEQ ID NO: 12)
DIQMTQSPSSLSASVGDRVTITCRAS
CDR1 of Variable Light Region:
(X4)ₙ
Framework Region 2 of Variable Light Region:
(SEQ ID NO: 13)
LNWYQQKPGKAPKLLIY
CDR2 of Variable Light Region:
(X5)ₙ
Framework Region 3 of Variable Light Region:
(SEQ ID NO: 14)
GVPSRFSGSGSGTDFTLTISSLQPEDAATYYC
CDR3 of Variable Light Region:
(X6)ₙ
Framework Region 4 of Variable Light Region:
(SEQ ID NO: 15)
FGGGTKVEIKR
Exemplary scFv structure
Variable Light Region + Linker (e.g., (G₄S);
linker) + Variable Heavy Region using CDRs
of Clone #1:
(SEQ ID NO: 45)
DIQMTQSPSSLSASVGDRVTITCRASQSISSYLNWYQQKPGKAPKLLIYAASSLQSGV
PSRFSGSGSGTDFTLTISSLQPEDAATYYCQQYDTFPLTFGGGTKVEIKRGGGGSGG
GGSGGGGSGGGGGGGGSQVQLVESGGGLVKPGGSLRLSCAASGFTFSDYYMSWI
RQAPGKGLEWVSYISSSGSTIYYADSVKGRFTISRDNAKNSLYLQMNSLRAEDTAV
YYCAREVYYYDSSGYYTYYFDYWGQGTLVTVSS
(SEQ ID NO: 46)
GATATTCAAATGACCCAGTCTCCTTCTTCCTTGAGCGCGTCTGTGGGAGACCGTG
TAACTATCACGTGTCGTGCATCCCAAAGCATTTCGAGTTACTTGAACTGGTATCA
ACAAAAGCCTGGTAAAGCGCCCAAACTGTTGATCTATGCTGCCAGTAGTCTTCAA
TCTGGTGTGCCCTCGCGCTTCTCAGGTTCGGGGTCAGGAACGGACTTTACCCTGA
CCATTAGCTCTCTGCAACCGGAAGATGCTGCAACGTATTACTGTCAACAATATGA
TACTTTTCCACTCACTTTCGGCGGAGGGACCAAAGTGGAGATCAAAGGAGGGGG
TGGCAGCGGTGGCGGGGGAAGTGGCGGTGGAGGGTCGGGGGGAGGCGGTTCCG
GGGGTGGAGGCTCACAAGTTCAATTAGTGGAGAGCGGCGGCGGATTGGTTAAAC
CGGGGGGCAGTCTGCGTTTAAGCTGCGCAGCTTCCGGGTTTACTTTTTCGGATTA
CTATATGAGCTGGATTCGTCAGGCTCCCGGTAAAGGGTTGGAATGGGTTAGCTAC
ATCTCAAGTTCCGGCTCTACAATCTACTATGCAGATTCCGTGAAAGGGCGCTTCA
CCATCTCTCGTGACAATGCAAAGAACAGTTTATACTTACAAATGAACTCGCTGCG
TGCGGAAGATACGGCCGTGTATTACTGTGCGAGAGAGGTATATTACTATGATAG
TAGTGGTTATTATACCTACTACTTTGACTACTGGGGCCAGGGAACCCTGGTCACT
GTCTCCTCA
Exemplary scFv structure
Variable Light Region + Linker (e.g., (G₄S)₃ linker) +
Variable Heavy Region using CDRs
of Clone #1:
(SEQ ID NO: 47)
DIQMTQSPSSLSASVGDRVTITCRASQSISSYLNWYQQKPGKAPKLLIYAASSLQSGV
PSRFSGSGSGTDFTLTISSLQPEDAATYYCQQYDTFPLTFGGGTKVEIKRGGGGSGG
GGSGGGGSQVQLVESGGGLVKPGGSLRLSCAASGFTFSDYYMSWIRQAPGKGLEW
VSYISSSGSTIYYADSVKGRFTISRDNAKNSLYLQMNSLRAEDTAVYYCAREVYYY
DSSGYYTYYFDYWGQGTLVTVSS
(SEQ ID NO: 48)
GATATTCAAATGACCCAGTCTCCTTCTTCCTTGAGCGCGTCTGTGGGAGACCGTG
TAACTATCACGTGTCGTGCATCCCAAAGCATTTCGAGTTACTTGAACTGGTATCA
ACAAAAGCCTGGTAAAGCGCCCAAACTGTTGATCTATGCTGCCAGTAGTCTTCAA
TCTGGTGTGCCCTCGCGCTTCTCAGGTTCGGGGTCAGGAACGGACTTTACCCTGA
CCATTAGCTCTCTGCAACCGGAAGATGCTGCAACGTATTACTGTCAACAATATGA
TACTTTTCCACTCACTTTCGGCGGAGGGACCAAAGTGGAGATCAAAGGAGGGGG
TGGCAGCGGTGGCGGGGGAAGTGGCGGTGGAGGGTCGCAAGTTCAATTAGTGGA
GAGCGGCGGCGGATTGGTTAAACCGGGGGGCAGTCTGCGTTTAAGCTGCGCAGC
TTCCGGGTTTACTTTTTCGGATTACTATATGAGCTGGATTCGTCAGGCTCCCGGTA
AAGGGTTGGAATGGGTTAGCTACATCTCAAGTTCCGGCTCTACAATCTACTATGC
AGATTCCGTGAAAGGGCGCTTCACCATCTCTCGTGACAATGCAAAGAACAGTTT
ATACTTACAAATGAACTCGCTGCGTGCGGAAGATACGGCCGTGTATTACTGTGCG
AGAGAGGTATATTACTATGATAGTAGTGGTTATTATACCTACTACTTTGACTACT
GGGGCCAGGGAACCCTGGTCACTGTCTCCTCA
Exemplary scFv structure
Variable Heavy Region + Linker (e.g., (G₄S), linker) +
Variable Light Region using CDRs
of Clone #1:
(SEQ ID NO: 49)
QVQLVESGGGLVKPGGSLRLSCAASGFTFSDYYMSWIRQAPGKGLEWVSYISSSGS
TIYYADSVKGRFTISRDNAKNSLYLQMNSLRAEDTAVYYCAREVYYYDSSGYYTY
YFDYWGQGTLVTVSSGGGGGGGGSGGGGSGGGGSGGGGSDIQMTQSPSSLSASVG
DRVTITCRASQSISSYLNWYQQKPGKAPKLLIYAASSLQSGVPSRFSGSGSGTDF
TLTISSLQPEDAATYYCQQYDTFPLTFGGGTKVEIKR
(SEQ ID NO: 50)
CAAGTTCAATTAGTGGAGAGCGGCGGCGGATTGGTTAAACCGGGGGGCAGTCTG
CGTTTAAGCTGCGCAGCTTCCGGGTTTACTTTTTCGGATTACTATATGAGCTGGAT
TCGTCAGGCTCCCGGTAAAGGGTTGGAATGGGTTAGCTACATCTCAAGTTCCGGC
TCTACAATCTACTATGCAGATTCCGTGAAAGGGCGCTTCACCATCTCTCGTGACA
ATGCAAAGAACAGTTTATACTTACAAATGAACTCGCTGCGTGCGGAAGATACGG
CCGTGTATTACTGTGCGAGAGAGGTATATTACTATGATAGTAGTGGTTATTATAC
CTACTACTTTGACTACTGGGGCCAGGGAACCCTGGTCACTGTCTCCTCAGGAGGG
GGTGGCAGCGGTGGCGGGGGAAGTGGCGGTGGAGGGTCGGGGGGAGGCGGTTC
CGGGGGTGGAGGCTCAGATATTCAAATGACCCAGTCTCCTTCTTCCTTGAGCGCG
TCTGTGGGAGACCGTGTAACTATCACGTGTCGTGCATCCCAAAGCATTTCGAGTT
ACTTGAACTGGTATCAACAAAAGCCTGGTAAAGCGCCCAAACTGTTGATCTATG
CTGCCAGTAGTCTTCAATCTGGTGTGCCCTCGCGCTTCTCAGGTTCGGGGTCAGG
AACGGACTTTACCCTGACCATTAGCTCTCTGCAACCGGAAGATGCTGCAACGTAT
TACTGTCAACAATATGATACTTTTCCACTCACTTTCGGCGGAGGGACCAAAGTGG
AGATCAAA
Exemplary scFv structure
Variable Heavy Region + Linker (e.g., (G₄S)₃
linker) + Variable Light Region using CDRs
of Clone #1:
(SEQ ID NO: 51)
QVQLVESGGGLVKPGGSLRLSCAASGFTFSDYYMSWIRQAPGKGLEWVSYISSSGS
TIYYADSVKGRFTISRDNAKNSLYLQMNSLRAEDTAVYYCAREVYYYDSSGYYTY
YFDYWGQGTLVTVSSGGGGGGGGSGGGGSDIQMTQSPSSLSASVGDRVTITCRAS
QSISSYLNWYQQKPGKAPKLLIYAASSLQSGVPSRFSGSGSGTDFTLTISSLQPE
DAATYYCQQYDTFPLTFGGGTKVEIKR
(SEQ ID NO: 52)
CAAGTTCAATTAGTGGAGAGCGGCGGCGGATTGGTTAAACCGGGGGGCAGTCTG
CGTTTAAGCTGCGCAGCTTCCGGGTTTACTTTTTCGGATTACTATATGAGCTGG
ATTCGTCAGGCTCCCGGTAAAGGGTTGGAATGGGTTAGCTACATCTCAAGTTCC
GGCTCTACAATCTACTATGCAGATTCCGTGAAAGGGCGCTTCACCATCTCTCGT
GACAATGCAAAGAACAGTTTATACTTACAAATGAACTCGCTGCGTGCGGAAGAT
ACGGCCGTGTATTACTGTGCGAGAGAGGTATATTACTATGATAGTAGTGGTTAT
TATACCTACTACTTTGACTACTGGGGCCAGGGAACCCTGGTCACTGTCTCCTCA
GGAGGGGGTGGCAGCGGTGGCGGGGGAAGTGGCGGTGGAGGGTCGGATATTCAAA
TGACCCAGTCTCCTTCTTCCTTGAGCGCGTCTGTGGGAGACCGTGTAACTATCAC
GTGTCGTGCATCCCAAAGCATTTCGAGTTACTTGAACTGGTATCAACAAAAGCCT
GGTAAAGCGCCCAAACTGTTGATCTATGCTGCCAGTAGTCTTCAATCTGGTGTGC
CCTCGCGCTTCTCAGGTTCGGGGTCAGGAACGGACTTTACCCTGACCATTAGCTC
TCTGCAACCGGAAGATGCTGCAACGTATTACTGTCAACAATATGATACTTTTCCA
CTCACTTTCGGCGGAGGGACCAAAGTGGAGATCAAA

### Example 8: Exemplary scFvs Having the Ability to Bind an ENPP1 Polypeptide

This Example provides the amino acid sequences of an exemplary heavy chain variable domain and an exemplary light chain variable domain of an exemplary scFv. The CDRs and framework sequences of each also are delineated. An exemplary linker amino acid sequence such as a linker amino acid sequence set forth in Example 11 can be used to link the heavy chain variable domain and the light chain variable domain together to form a scFv. The structures of exemplary scFv's including the amino acid and nucleic acid sequences with the linkers, CDRs, and framework sequences delineated are shown.

Exemplary scFv heavy chain variable domain
Variable Heavy Region:
QVQLVESGGGLVKPGGSLRLSCAAS(X1)ₙMSWIRQAPGKGLEWVSY
(X2)ₙYYADSVKGRFTISRDNAKNSLYLQMNSLRAEDTAVYYC(X3)ₙ
WGQGTLVTVSS (SEQ ID NO: 53 which includes SEQ ID
NO: 4, followed by a CDR1 of a variable heavy
region, followed by SEQ ID NO: 5, followed by a
CDR2 of a variable heavy region, followed by SEQ
ID NO: 22, followed by a CDR3 of a variable
heavy region, followed by SEQ ID NO: 7)
Framework Region 1 of Variable Heavy
Region:
(SEQ ID NO: 4)
QVQLVESGGGLVKPGGSLRLSCAAS
CDR1 of Variable Heavy Region:
(X1)ₙ
Framework Region 2 of Variable Heavy
Region:
(SEQ ID NO: 5)
MSWIRQAPGKGLEWVSY
CDR2 of Variable Heavy Region:
(X2)ₙ
Framework Region 3 of Variable Heavy
Region:
(SEQ ID NO: 22)
YYADSVKGRFTISRDNAKNSLYLQMNSLRAEDTAVYYC
CDR3 of Variable Heavy Region:
(X3)ₙ
Framework Region 4 of Variable Heavy
Region:
(SEQ ID NO: 7)
WGQGTLVTVSS
Exemplary scFv light chain variable domain
Variable Light Region:
DIQMTQSPSSLSASVGDRVTITCRAS(X4)ₙLNWYQQKPGKAPKLLIY
(X5)ₙGVPSRFSGSGSGTDFTLTISSLQPEDVAVYYC(X6)ₙFGQGTK
LEIKR (SEQ ID NO: 54 which includes SEQ ID NO:
12, followed by a CDR1 of a variable light
region, followed by SEQ ID NO: 13, followed by a
CDR2 of a variable light region, followed by SEQ
ID NO: 30, followed by a CDR3 of a variable
light region, followed by SEQ ID NO: 31)
Framework Region 1 of Variable Light
Region:
(SEQ ID NO: 12)
DIQMTQSPSSLSASVGDRVTITCRAS
CDR1 of Variable Light Region:
(X4)ₙ
Framework Region 2 of Variable Light
Region:
(SEQ ID NO: 13)
LNWYQQKPGKAPKLLIY
CDR2 of Variable Light Region:
(X5)ₙ
Framework Region 3 of Variable Light
Region:
(SEQ ID NO: 30)
GVPSRFSGSGSGTDFTLTISSLQPEDVAVYYC
CDR3 of Variable Light Region:
(X6)ₙ
Framework Region 4 of Variable Light
Region:
(SEQ ID NO: 31)
FGQGTKLEIKR
Exemplary scFv structures
Variable Light Region + Linker (e.g.,
(G₄S)₃ linker) + Variable Heavy Region
using CDRs of Clone #2:
(SEQ ID NO: 55)
DIQMTQSPSSLSASVGDRVTITCRASQSISSYLNWYQQKPGKAPKLLIYA
ASSLQSGVPSRFSGSGSGTDFTLTISSLQPEDAATYYCQQYDTFPLTFGG
GTKVEIKRGGGGSGGGGSGGGGSQVQLVESGGGLVKPGGSLRLSCAASGF
TFSDYYMSWIRQAPGKGLEWVSYISSSGSTIYYADSVKGRFTISRDNAKN
SLYLQMNSLRAEDTAVYYCAREVYYYDSSGYYTYYFDYWGQGTLVTVSS
Variable Heavy Region + Linker (e.g., (G₄S)₃
linker) + Variable Light Region using CDRs of
Clone #2:
(SEQ ID NO: 56)
QVQLVESGGGLVKPGGSLRLSCAASGFTFSDYYMSWIRQAPGKGLEWVSY
ISSSGSTIYYADSVKGRFTISRDNAKNSLYLQMNSLRAEDTAVYYCAREV
YYYDSSGYYTYYFDYWGQGTLVTVSSGGGGSGGGGGGGGSDIQMTQSPSS
LSASVGDRVTITCRASQSISSYLNWYQQKPGKAPKLLIYAASSLQSGVPS
RFSGSGSGTDFTLTISSLQPEDAATYYCQQYDTFPLTFGGGTKVEIKR

### Example 9: Exemplary scFvs Having the Ability to Bind an ENPP1 Polypeptide

This Example provides the amino acid sequences of an exemplary heavy chain variable domain and an exemplary light chain variable domain of an exemplary scFv. The CDRs and framework sequences of each also are delineated. An exemplary linker amino acid sequence such as a linker amino acid sequence set forth in Example 11 can be used to link the heavy chain variable domain and the light chain variable domain together to form a scFv.

Exemplary scFv heavy chain variable domain
Variable Heavy Region:
EVQLVQSGGGLVQPGGSLRLSCSAS(X1)ₙMHWVRQAPGKGLEYVSA
(X2)ₙYYADSVKGRFTISRDNSKNTLYLQMNSLRAEDTAVYYC(X3)ₙ
NWGQGTMVTVSS (SEQ ID NO: 57 which includes SEQ
ID NO: 58, followed by a CDR1 of a variable
heavy region, followed by SEQ ID NO: 59),
followed by a CDR2 of a variable heavy region,
followed by SEQ ID NO: 60, followed by a CDR3 of
a variable heavy region, followed by SEQ ID
NO: 61)
Framework Region 1 of Variable Heavy Region:

(SEQ ID NO: 58)

EVQLVQSGGGLVQPGGSLRLSCSAS
CDR1 of Variable Heavy Region:
(X1)ₙ
Framework Region 2 of Variable Heavy Region:

(SEQ ID NO: 59)

MHWVRQAPGKGLEYVSA
CDR2 of Variable Heavy Region:
(X2)ₙ
Framework Region 3 of Variable Heavy Region:

(SEQ ID NO: 60)

YYADSVKGRFTISRDNSKNTLYLQMNSLRAEDTAVYYC
CDR3 of Variable Heavy Region:
(X3)ₙ
Framework Region 4 of Variable Heavy Region:

(SEQ ID NO: 61)

WGQGTMVTVSS
Exemplary scFv light chain variable domain
Variable Light Region:
EATLTQSPSSLSASVGDRVTITCRAS(X4)ₙLNWYQQKPGRAPKLLI
Y(X5)ₙSLQSGVPSRFSGSGSGTDFTLTISSLQPEDFATYYC(X6)ₙ
FGGGTKVEIKR (SEQ ID NO: 62 which includes SEQ
ID NO: 63, followed by a CDR1 of a variable
light region, followed by SEQ ID NO: 64,
followed by a CDR2 of a variable light region,
followed by SEQ ID NO: 65, followed by a CDR3
of a variable light region, followed by SEQ ID
NO: 15)
Framework Region 1 of Variable Light Region:

(SEQ ID NO: 63)

EATLTQSPSSLSASVGDRVTITCRAS
CDR1 of Variable Light Region:
(X4)ₙ
Framework Region 2 of Variable Light Region:

(SEQ ID NO: 64)

LNWYQQKPGRAPKLLIY
CDR2 of Variable Light Region:
(X5)ₙ
Framework Region 3 of Variable Light Region:

(SEQ ID NO: 65)

SLQSGVPSRFSGSGSGTDFTLTISSLQPEDFATYYC
CDR3 of Variable Light Region:
(X6)ₙ
Framework Region 4 of Variable Light Region:

(SEQ ID NO: 15)

FGGGTKVEIKR

### Example 10: Exemplary scFvs Having the Ability to Bind an ENPP1 Polypeptide

This Example provides the amino acid sequences of an exemplary heavy chain variable domain and an exemplary light chain variable domain of an exemplary scFv. The CDRs and framework sequences of each also are delineated. An exemplary linker amino acid sequence such as a linker amino acid sequence set forth in Example 11 can be used to link the heavy chain variable domain and the light chain variable domain together to form a scFv.

Exemplary scFv heavy chain variable domain
Variable Heavy Region:
EVQLVQSGGGLVQPGGSLRLSCAAS(X1)ₙMTWVRQAPGKGLEWVST
(X2)ₙYYADSVKGRFTISRDNDKNTLYLQMNSLRADDTAVYYC(X3)ₙ
WGQGTLVTVSS (SEQ ID NO: 66 which includes SEQ ID
NO: 67, followed by a CDR1 of variable heavy
region, followed by SEQ ID NO: 68, followed by a
CDR2 of variable heavy region, followed by SEQ
ID NO: 69, followed by a CDR3 of variable heavy
region, followed by SEQ ID NO: 70)
Framework Region 1 of Variable Heavy Region:

(SEQ ID NO: 67)

EVQLVQSGGGLVQPGGSLRLSCAAS
CDR1 of Variable Heavy Region:
(X1)ₙ
Framework Region 2 of Variable Heavy Region:

(SEQ ID NO: 68)

MTWVRQAPGKGLEWVST
CDR2 of Variable Heavy Region:
(X2)ₙ
Framework Region 3 of Variable Heavy Region:

(SEQ ID NO: 69)

YYADSVKGRFTISRDNDKNTLYLQMNSLRADDTAVYYC
CDR3 of Variable Heavy Region:
(X3)ₙ
Framework Region 4 of Variable Heavy Region:

(SEQ ID NO: 70)

WGQGTLVTVSS
Exemplary scFv light chain variable domain
Variable Light Region:
QSVVTQPPSASGSPGQSVTISCTGT(X4)ₙVSWYQQHPGKAPKLLIY
(X5)ₙERPSGVPDRFSGSKSDNKASLTIIGLQTEDEADYYC(X6)ₙF
GTGTKLTVLR (SEQ ID NO: 71 which includes SEQ ID
NO: 72, followed by a CDR1 of a variable light
region, followed by SEQ ID NO: 73, followed by
a CDR2 of a variable light region, followed by
SEQ ID NO: 74, followed by a CDR3 of a variable
light region, followed by SEQ ID NO: 75)
Framework Region 1 of Variable Light Region:

(SEQ ID NO: 72)

QSVVTQPPSASGSPGQSVTISCTGT
CDR1 of Variable Light Region:
(X4)ₙ
Framework Region 2 of Variable Light Region:

(SEQ ID NO: 73)

VSWYQQHPGKAPKLLIY
CDR2 of Variable Light Region:
(X5)ₙ
Framework Region 3 of Variable Light Region:

(SEQ ID NO: 74)

ERPSGVPDRFSGSKSDNKASLTIIGLQTEDEADYYC
CDR3 of Variable Light Region:
(X6)ₙ
Framework Region 4 of Variable Light Region:

(SEQ ID NO: 75)

FGTGTKLTVLR

### Example 11: Exemplary Linkers

This Example provides exemplary linker amino acid sequences that can be used to link a heavy chain variable domain and a light chain variable domain together to form a scFv. These linker sequences also can be used to create CARs and cell engagers.

Exemplary Linker Sequences for scFv's,
CARs, and/or cell engagers:

| | |
|---|---|
| a) | GGGGSGGSGSGGGGS (SEQ ID NO: 76) |
| b) | GGGGSGGSGSSGGGS (SEQ ID NO: 77) |
| c) | GGGGSGGGGSGGGGS (SEQ ID NO: 78) |
| d) | GGGGSGSGASSGGGS (SEQ ID NO: 79) |
| e) | GGGGSGSGASGGGGS (SEQ ID NO: 80) |
| f) | SGGGSGGGGSGGGGS (SEQ ID NO: 81) |
| g) | GSTSGSGKPGSGEGSTKG (SEQ ID NO: 82) |
| h) | PNGASQSSSASHTGSAPGS (SEQ ID NO: 83) |
| i) | PNGASNSGSAPDTSSAPGS (SEQ ID NO: 84) |
| j) | PNGASHSGSAPNTSSAPGS (SEQ ID NO: 85) |
| k) | PNGASESGSASKTSSASGS (SEQ ID NO: 86) |
| 1) | PNGASNSGSAPKTGSASGS (SEQ ID NO: 87) |
| m) | PNGASKSGSASQTSSAPGS (SEQ ID NO: 88) |
| n) | PNGASHSSSASQTGSAPGS (SEQ ID NO: 89) |
| o) | PNGASKRSAPGS (SEQ ID NO: 90) |
| p) | PNGASHSGSAPHTSSASGS (SEQ ID NO: 91) |
| q) | RGRGRGRGRSRGGGS (SEQ ID NO: 92) |
| r) | SHGGSHGGGSGGGGS (SEQ ID NO: 93) |
| s) | GQAGR (SEQ ID NO: 94) |
| t) | GGGSGGGG (SEQ ID NO: 95) |
| u) | SGGGG (SEQ ID NO: 96) |
| v) | GGGGS (SEQ ID NO: 97) |
| w) | GGGGSGGGGSGGGGSGGGGSGGGGSGGGGS (SEQ |
| | ID NO: 98) |
| x) | GGGGGSGGGGSGGGGS (SEQ ID NO: 99) |
| y) | GGGGGSGGGGSGGGGSGGGGSGGGGS (SEQ ID |
| | NO: 100) |
| z) | GGGGSGGGGGGGGSGGGGSGGGGS (SEQ ID |
| | NO: 101) |
| a1) | AAA (SEQ ID NO: 102) |

### Example 12: Exemplary CAR Structures

This Example provides the structures of exemplary CARs.

### Exemplary CAR Structures:

Signal Peptide+scFv+(Linker/Hinge)ₙ₁+Transmembrane Domain+(Intracellular Signaling Domain)ₙ₂
n1=0, 1, 2, or 3
n2=1, 2, 3, 4, or 5 with or without a linker located between consecutive intracellular signaling domains
Signal Peptide+scFv+Linker+Hinge+Transmembrane Domain+Intracellular Signaling Domain+Intracellular Signaling Domain+Linker+Intracellular Signaling Domain
Signal Peptide+scFv+Linker+Hinge+Transmembrane Domain+Intracellular Signaling Domain+Linker+Intracellular Signaling Domain
Signal Peptide+scFv+Hinge+Transmembrane Domain+Intracellular Signaling Domain+Intracellular Signaling Domain+Linker+Intracellular Signaling Domain
Signal Peptide+scFv+Hinge+Transmembrane Domain+Intracellular Signaling Domain+Intracellular Signaling Domain+Intracellular Signaling Domain
Signal Peptide+scFv+Linker+Transmembrane Domain+Intracellular Signaling Domain+Intracellular Signaling Domain+Intracellular Signaling Domain

### Example 13: Exemplary Signal Peptides

This Example provides the amino acid sequences of exemplary signal peptides that can be used to design a CAR.

Exemplary signal peptides for CARS
Exemplary human IGKV1-39-derived signal
peptide:
(SEQ ID NO: 103)
MDMRVPAQLLGLLLLWLRGARC
(SEQ ID NO: 104)
ATGGATATGAGGGTCCCCGCACAATTGCTCGGTCTCCTTCTGCTTTGGCT
CCGGGGCGCGCGGTGC
Exemplary human IGKV1-16-derived signal
peptide:
(SEQ ID NO: 155)
MDMRVLAQLLGLLLLCFPGARC
Exemplary human IGKV3-11-derived signal
peptide:
(SEQ ID NO: 156)
MEAPAQLLFLLLLWLPDTTG
Exemplary human IGKV4-1-derived signal
peptide:
(SEQ ID NO: 157)
MVLQTQVFISLLLWISGAYG
Exemplary human IGKV6-21-derived signal
peptide:
(SEQ ID NO: 158)
MLPSQLIGFLLLWVPASRG
Exemplary human IGKV1-33-derived signal
peptide:
(SEQ ID NO: 159)
MDMRVPAQLLGLLLLWLSGARC

### Example 14: Exemplary Hinges

This Example provides the amino acid sequences of exemplary hinges that can be used to design a CAR.

Exemplary hinges for CARS
Exemplary human IgG1-derived hinge:
(SEQ ID NO: 105)
EPKSCDKTHTCPPCP
Exemplary human IgG2-derived hinge:
(SEQ ID NO: 106)
DKTHTCPPCPAPPVA
Exemplary human IgG4-derived hinges:
(SEQ ID NO: 107)
ESKYGPPCPPCP
(SEQ ID NO: 108)
ESKYGPPCPSCP
Exemplary human CD8α-derived hinge:
(SEQ ID NO: 109)
KPTTTPAPRPPTPAPTIASQPLSLRPEACRPAAGGAVHTRGLDFACDIY
Exemplary human CD28-derived hinges:
(SEQ ID NO: 110)
IEVMYPPPYLDNERSNGTIIHVKGKHLCPSPLFPGPSKP
(SEQ ID NO: 111)
TTTPAPRPPTPAPTIASQPLSLRPEACRPAAGGAVHTRGLDFACDIY
(SEQ ID NO: 112)
TTTPAPRPPTPAPTIASQPLSLRPEACRPAAGGAVHTRGLDFACD

### Example 15: Exemplary Transmembrane Domains

This Example provides the amino acid sequences of exemplary transmembrane domains that can be used to design a CAR.

Exemplary transmembrane domains for CARS
Exemplary human CD3ζ transmembrane domain:

(SEQ ID NO: 113)

LCYLLDGILFIYGVILTALFL
Exemplary human CD4 transmembrane domain:

(SEQ ID NO: 114)

MALIVLGGVAGLLLFIGLGIFF
Exemplary human CD8α transmembrane domains:

(SEQ ID NO: 115)

IYIWAPLAGTCGVLLLSLVIT

(SEQ ID NO: 116)

IYIWAPLAGTCGVLLLSLVITLYC

(SEQ ID NO: 117)

IWAPLAGTCGVLLLSLVITLYC

(SEQ ID NO: 118)

IWAPLAGTCGVLLLSLVIT
Exemplary human CD28 transmembrane domain:

(SEQ ID NO: 119)

FWVLVVVGGVLACYSLLVTVAFIIFWV
Exemplary human CD278 transmembrane domain:

(SEQ ID NO: 120)

FWLPIGCAAFVVVCILGCILI

### Example 16: Exemplary Intracellular Signaling Domains

This Example provides the amino acid sequences of exemplary intracellular signaling domains that can be used to design a CAR.

Exemplary intracellular signaling
domains for CARS
Exemplary human CD3ζ intracellular
signaling domain:
(SEQ ID NO: 121)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR
Exemplary human 4-1BB (CD137)
intracellular signaling domains:
(SEQ ID NO: 122)
KRGRKKLLYIFKQPFMRPVQTTQEEDGCSCRFPEEEEGGCEL
(SEQ ID NO: 123)
KRGRKKLLYIFKQPFMRPVQTTQEEDGCSCRFPEEEE
Exemplary human CD28 intracellular
signaling domain:
(SEQ ID NO: 124)
RSKRSRLLHSDYMNMTPRRPGPTRKHYQPYAPPRDFAAYRS
Exemplary human OX40 (CD134) intracellular
signaling domain:
(SEQ ID NO: 125)
ALYLLRRDQRLPPDAHKPPGGGSFRTPIQEEQADAHSTLAKI
Exemplary human CD278 intracellular
signaling domain:
(SEQ ID NO: 126)
CWLTKKKYSSSVHDPNGEYMFMRAVNTAKKSRLTDVTL
Exemplary human DAP10 intracellular
signaling domain:
(SEQ ID NO: 127)
LCARPRRSPAQEDGKVYINMPGRG
Exemplary human DAP12 intracellular
signaling domain:
(SEQ ID NO: 128)
YFLGRLVPRGRGAAEAATRKQRITETESPYQELQGQRSDVYSDLNTQRPY
YK
Exemplary human CD27 intracellular
signaling domain:
(SEQ ID NO: 129)
QRRKYRSNKGESPVEPAEPCHYSCPREEEGSTIPIQEDYRKPEPACSP

### Example 17: Exemplary CARs Having the Ability to Bind an ENPP1 Polypeptide

This Example provides an amino acid sequence of a CAR (CAR #1) designed to include a scFv created using the CDRs of the Clone #1 Fab and a nucleic acid sequence encoding that CAR. The various components of this CAR (e.g., domains and linkers) are provided and delineated.

CAR designed using CDRs of Clone #1:
GM-CSF Signal Peptide + scFv of Example 7 +
Linker + CD8A + CD8A Transmembrane Domain +
4-1BB Intracellular Signaling Domain +
Linker + CD3ζ Intracellular Signaling
Domain
(SEQ ID NO: 130)
MLLLVTSLLLCELPHPAFLLIPDTSAQAADIQMTQSPSSLSASVGDRVTI
TCRASQSISSYLNWYQQKPGKAPKLLIYAASSLQSGVPSRFSGSGSGTDF
TLTISSLQPEDAATYYCQQYDTFPLTFGGGTKVEIKRGGGGSGGGGSGGG
GSQVQLVESGGGLVKPGGSLRLSCAASGFTFSDYYMSWIRQAPGKGLEWV
SYISSSGSTIYYADSVKGRFTISRDNAKNSLYLQMNSLRAEDTAVYYCAR
EVYYYDSSGYYTYYFDYWGQGTLVTVSSGQAGRTTTPAPRPPTPAPTIAS
QPLSLRPEACRPAAGGAVHTRGLDFACDIYIWAPLAGTCGVLLLSLVITL
YCKRGRKKLLYIFKQPFMRPVQTTQEEDGCSCRFPEEEEGGCELRVKFSR
SADAPAYQQGONQLYNELNLGRREEYDVLDKRRGRDPEMGGKPQRRKNPQ
EGLYNELQKDKMAEAYSEIGMKGERRRGKGHDGLYQGLSTATKDTYDALH
MQALPPR
(SEQ ID NO: 131)
ATGCTTCTGCTCGTGACAAGCCTGCTGCTGTGCGAGCTGCCCCACCCTGC
CTTTCTGCTGATCCCTGACACCTCGGCCCAGGCGGCCGATATTCAAATGA
CCCAGTCTCCTTCTTCCTTGAGCGCGTCTGTGGGAGACCGTGTAACTATC
ACGTGTCGTGCATCCCAAAGCATTTCGAGTTACTTGAACTGGTATCAACA
AAAGCCTGGTAAAGCGCCCAAACTGTTGATCTATGCTGCCAGTAGTCTTC
AATCTGGTGTGCCCTCGCGCTTCTCAGGTTCGGGGTCAGGAACGGACTTT
ACCCTGACCATTAGCTCTCTGCAACCGGAAGATGCTGCAACGTATTACTG
TCAACAATATGATACTTTTCCACTCACTTTCGGCGGAGGGACCAAAGTGG
AGATCAAAGGAGGGGGTGGCAGCGGTGGCGGGGGAAGTGGCGGTGGAGGG
TCGCAAGTTCAATTAGTGGAGAGCGGCGGCGGATTGGTTAAACCGGGGGG
CAGTCTGCGTTTAAGCTGCGCAGCTTCCGGGTTTACTTTTTCGGATTACT
ATATGAGCTGGATTCGTCAGGCTCCCGGTAAAGGGTTGGAATGGGTTAGC
TACATCTCAAGTTCCGGCTCTACAATCTACTATGCAGATTCCGTGAAAGG
GCGCTTCACCATCTCTCGTGACAATGCAAAGAACAGTTTATACTTACAAA
TGAACTCGCTGCGTGCGGAAGATACGGCCGTGTATTACTGTGCGAGAGAG
GTATATTACTATGATAGTAGTGGTTATTATACCTACTACTTTGACTACTG
GGGCCAGGGAACCCTGGTCACTGTCTCCTCAGGCCAGGCCGGCCGCACCA
CGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGT
GCACACGAGGGGGCTGGACTTCGCCTGTGATATCTACATCTGGGCGCCCT
TGGCCGGGACTTGTGGGGTCCTTCTCCTGTCACTGGTTATCACCCTTTAC
TGCAAACGGGGCAGAAAGAAACTCCTGTATATATTCAAACAACCATTTAT
GAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGC
GCAGACGCCCCCGCGTACAAGCAGGGCCAGAACCAGCTCTATAACGAGCT
CAATCTAGGACGAAGAGAGGAGTACGATGTTTTGGACAAGAGACGTGGCC
GGGACCCTGAGATGGGGGGAAAGCCGAGAAGGAAGAACCCTCAGGAAGGC
CTGTACAATGAACTGCAGAAAGATAAGATGGCGGAGGCCTACAGTGAGAT
TGGGATGAAAGGCGAGCGCCGGAGGGGCAAGGGGCACGATGGCCTTTACC
AGGGTCTCAGTACAGCCACCAAGGACACCTACGACGCCCTTCACATGCAG
GCCCTGCCCCCTCGCTAA

### Example 18: Exemplary BITEs Having the Ability to Bind an ENPP1 Polypeptide

This Example provides the amino acid sequences of exemplary antigen binding domains that can be used to design cell engagers that bind to T cells. For example, this Example provides an amino acid sequence of a linker sequence (SEQ ID NO: 162; nucleic acid sequence of the linker is SEQ ID NO: 163) followed by an gOKT3-7 scFv sequence, which can be attached to a light chain as shown in FIG. 9. This Example also depicts a nucleic acid sequence encoding that linker and gOKT3-7 scFv.

Linker + gOKT3-7 (anti-CD3 scFv)
(SEQ ID NO: 153)
GGGGSGGGGSGGGGSQVQLVQSGGGVVQPGRSLRLSCKASGYTFTRYTMH
WVRQAPGKGLEWIGYINPSRGYTNYNQKVKDRFTISRDNSKNTAFLQMDS
LRPEDTGVYFCARYYDDHYCLDYWGQGTPVTVSSGGGGSGGGGSGGGGSG
GGGSGGGGSGGGGSDIQMTQSPSSLSASVGDRVTITCSASSSVSYMNWYQ
QTPGKAPKRWIYDTSKLASGVPSRFSGSGSGTDYTFTISSLQPEDIATYY
CQQWSSNPFTFGQGTKLQITR
(SEQ ID NO: 154)
GGAGGTGGCGGGTCTGGTGGAGGCGGAAGCGGTGGTGGCGGATCCCAGGT
CCAACTGGTGCAAAGTGGAGGCGGCGTAGTACAGCCCGGTCGGTCCCTGC
GGCTCAGCTGTAAAGCAAGTGGGTACACTTTCACCAGATATACAATGCAC
TGGGTTAGACAGGCCCCCGGCAAGGGTCTTGAATGGATAGGATATATCAA
CCCATCCCGCGGATATACAAACTACAATCAGAAGGTGAAGGACCGGTTCA
CGATTAGTAGAGACAACTCCAAAAACACTGCGTTCCTTCAAATGGACTCA
TTGCGACCCGAGGACACTGGAGTGTATTTCTGTGCCAGGTATTATGACGA
CCATTATTGCCTGGACTACTGGGGGCAAGGCACACCGGTTACGGTATCTT
CCGGAGGTGGCGGGTCTGGTGGAGGCGGAAGCGGTGGTGGCGGATCCGGG
GGAGGAGGCTCTGGCGGAGGCGGATCAGGGGGTGGAGGTTCCGACATTCA
AATGACACAATCCCCTAGTTCTTTGTCTGCGTCCGTGGGGGATCGGGTAA
CAATCACTTGCTCTGCATCAAGCAGTGTTTCTTATATGAACTGGTATCAG
CAGACACCCGGTAAGGCCCCAAAACGATGGATTTATGACACGAGTAAACT
CGCATCCGGGGTGCCTTCACGCTTCTCCGGTTCTGGTTCCGGCACGGATT
ACACTTTCACCATCAGTAGCCTGCAACCTGAGGACATTGCAACTTATTAC
TGTCAACAATGGAGCAGTAATCCATTCACGTTCGGCCAAGGGACAAAACT
GCAGATTACTAGG

### Example 19: Exemplary Antigen Binding Domains Having the Ability to Bind a T Cell

This Example provides the amino acid sequences of exemplary antigen binding domains that can be used to design cell engagers that bind to T cells.

Exemplary antigen binding domains that bind
to T cells
Exemplary anti-human CD3 scFv (clone OKT3):
Heavy chain:
(SEQ ID NO: 132)
QVQLVQSGGGVVQPGRSLRLSCKASGYTFTRYTMHWVRQAPGKGLEWIGY
INPSRGYTNYNQKVKDRFTISRDNSKNTAFLQMDSLRPEDTGVYFCARYY
DDHYCLDYWGQGTPVTVSS
Light chain:
(SEQ ID NO: 133)
DIQMTQSPSSLSASVGDRVTITCSASSSVSYMNWYQQTPGKAPKRWIYDT
SKLASGVPSRFSGSGSGTDYTFTISSLQPEDIATYYCQQWSSNPFTFGQG
TKLQITR
Exemplary anti-human CD3 scFv (clone UCHT1):
Heavy chain:
(SEQ ID NO: 134)
EVQLQQSGPELVKPGASMKISCKASGYSFTGYTMNWVKQSHGKNLEWMGL
INPYKGVSTYNQKFKDKATLTVDKSSSTAYMELLSLTSEDSAVYYCARSG
YYGDSDWYFDVWGQGTTLTVFS
Light chain:
(SEQ ID NO: 135)
MDIQMTQTTSSLSASLGDRVTISCRASQDIRNYLNWYQQKPDGTVKLLIY
YTSRLHSGVPSKFSGSGSGTDYSLTISNLEQEDIATYFCQQGNTLPWTFA
GGTKLEIK

### Example 20: Exemplary Antigen Binding Domains Having the Ability to Bind a NK Cell

This Example provides the amino acid sequences of exemplary antigen binding domains that can be used to design cell engagers that bind to NK cells and the amino acid and nucleic acid sequence for an exemplary BiKE.

Exemplary antigen binding domains that
bind to NK cells
Exemplary anti-human CD16a scFv:
Heavy chain:
(SEQ ID NO: 136)
EVQLVESGGGVVRPGGSLRLSCAASGFTFDDYGMSWVRQAPGKGLEWVSG
INWNGGSTGYADSVKGRFTISRDNAKNSLYLQMNSLRAEDTAVYYCARGR
SLLFDYWGQGTLVTVSR
Light chain:
(SEQ ID NO: 137)
SSELTQDPAVSVALGQTVRITCQGDSLRSYYASWYQQKPGQAPVLVIYGK
NNRPSGIPDRFSGSSSGNTASLTITGAQAEDEADYYCNSRDSSGNHVVFG
GGTKLTVG
Exemplary anti-human NKG2A scFv:
Heavy chain:
(SEQ ID NO: 160)
QVQLVQSGAEVKKPGASVKVSCKASGYTFTSYWMNWVRQAPGQGLEWMGR
IDPYDSETHYAQKLQGRVTMTTDTSTSTAYMELRSLRSDDTAVYYCARGG
YDFDVGTLYWFFDVWGQGTTVTVSS
Light chain:
(SEQ ID NO: 161)
DIQMTQSPSSLSASVGDRVTITCRASENIYSYLAWYQQKPGKAPKLLIYN
AKTLAEGVPSRFSGSGSGTDFTLTISSLQPEDFATYYCQHHYGTPRTFGG
GTKVEIK
Exemplary anti-human NKG2A scFv:
Heavy chain:
(SEQ ID NO: 138)
EVQLVESGGGLVKPGGSLRLSCAASGFTFSSYAMSWVRQAPGKGLEWVSE
ISSGGSYTYYADSVKGRFTISRDNAKNSLYLQMNSLRAEDTAVYYCARHG
DYPRFFDVWGQGTTVTVSS
Light chain:
(SEQ ID NO: 139)
EIVLTQSPATLSLSPGERATLSCSASSSVSSYIYWYQQKPGQAPRLLIYL
TSNLASGIPARFSGSGSGTDFTLTISSLEPEDFAVYYCQQWSGNPYTFGQ
GTKLEIK
Exemplary anti-human NKG2D scFv:
Heavy chain:
(SEQ ID NO: 140)
QVQLVESGGGLVKPGGSLRLSCAASGFTFSSYGMHWVRQAPGKGLEWVAF
IRYDGSNKYYADSVKGRFTISRDNSKNTLYLQMNSLRAEDTAVYYCAKDR
GLGDGTYFDYWGQGTTVTVSS
Light chain:
(SEQ ID NO: 141)
QSALTQPASVSGSPGQSITISCSGSSSNIGNNAVNWYQQLPGKAPKLLIY
YDDLLPSGVSDRFSGSKSGTSAFLAISGLQSEDEADYYCAAWDDSLNGPV
FGGGTKLTVL
Exemplary anti-human NKp30 scFv:
Alternative heavy chains with underline
indicating the differences:
(SEQ ID NO: 142)
QVQLVQSGAEVKKPGASVKVSCKASGHTFTSYFMHWVRQAPGQGLEWMGI
INPSDDYANYAQKFQGRVTMTRDTSTSTVYMELSSLRSEDTAVYYCATAI
FDYWGQGTLVTVSS
or
(SEQ ID NO: 143)
QVQLVQSGAEVKKPGASVKVSCKASGHTFTSYFMHWVRQAPGQGLEWMGI
INPSDDYANYAQKFQGRVTMTRDTSTSTVYMELSSLRSEDTAVYYCATAI
FDYWGQGTPVTVSS
Light chain:
(SEQ ID NO: 144)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPLTFGG
GTKVEIK
Exemplary anti-human NKp46 scFv
Heavy chain:
(SEQ ID NO: 145)
EIQLQQSGAELVKPGASVKLSCTASGFNIKDTYFHWVKQRPEQGLEWIGR
IDPANGNTKYDPKFHDKATIIADISSNTAYLQFSSLTSEDTAVYYCAANR
YGYWGQGTTLTVSS
Light chain:
(SEQ ID NO: 146)
DIVMTQAAPSIPVTPGESVSISCRSSKSLLYINGNTHLFWFLQRPGQSPQ
LLIYRMSNLASGVPDRFSGSGSGTAFTLRISRVEAEDVGVYYCMQHLEYP
FTFGSGTKLEIK
Exemplary anti-human NKp46 scFy
Heavy chain:
(SEQ ID NO: 147)
QVQLQQSGPELVKPGASVKMSCKASGYTFTDYVINWGKQRSGQGLEWIGE
IYPGSGTNYYNEKFKAKATLTADKSSNIAYMQLSSLTSEDSAVYFCARRG
RYGLYAMDYWGQGTSVTVSS
Light chain:
(SEQ ID NO: 148)
DIQMTQTTSSLSASLGDRVTISCRASQDISNYLNWYQQKPDGTVKLLIYY
TSRLHSGVPSRFSGSGSGTDYSLTINNLEQEDIATYFCQQGNTRPWTFGG
GTKLEIK
Exemplary anti-human CD16a VH domain:
(SEQ ID NO: 149)
EVQLVESGGGLVQPGGSLRLSCAASGFTFSNYGMSWVRQAPGKGLEWIGS
IYYSGSTNYNPSLKSLVTISRDNSKNTLYLQMNSLRAEDTATYYCARESI
DYWGQGTLVTVSS
Exemplary BiKE that binds to ENPP1 and
NK cells:
17-Fab (Clone #1) VL + (G₄S)₃ linker
17-Fab (Clone #1) VH + (G₄S) linker
Anti-NKG2A VH + linker + Anti-NKG2A VL
6xHis-tag
(SEQ ID NO: 164)
DIQMTQSPSSLSASVGDRVTITCRASQSISSYLNWYQQKPGKAPKLLIYA
ASSLQSGVPSRFSGSGSGTDFTLTISSLQPEDAATYYCQQYDTFPLTFGG
GTKVEIKRGGGGSGGGGSGGGGSQVQLVESGGGLVKPGGSLRLSCAASGF
TFSDYYMSWIRQAPGKGLEWVSYISSSGSTIYYADSVKGRFTISRDNAKN
SLYLQMNSLRAEDTAVYYCAREVYYYDSSGYYTYYFDYWGQGTLVTVSSG
GGGSQVQLVQSGAEVKKPGASVKVSCKASGYTFTSYWMNWVRQAPGQGLE
WMGRIDPYDSETHYAQKLQGRVTMTTDTSTSTAYMELRSLRSDDTAVYYC
ARGGYDFDVGTLYWFFDVWGQGTTVTVSSVEGGSGGSGGSGGSGGVDDIQ
MTQSPSSLSASVGDRVTITCRASENIYSYLAWYQQKPGKAPKLLIYNAKT
LAEGVPSRFSGSGSGTDFTLTISSLQPEDFATYYCQHHYGTPRTFGGGTK
VEIKHHHHHH

### Example 21: Treating Cancer

Cells (e.g., T cells) designed to express one or more binders (e.g., antibodies, antigen binding fragments, antibody domains, CARs, or cell engagers) having the ability to bind to an ENPP1 polypeptide are administered to a human identified as having a sarcoma. The cells (e.g., T cells) engineered to express one or more binders (e.g., antibodies, antigen binding fragments, antibody domains, CARs, or cell engagers) having the ability to bind to an ENPP1 polypeptide are administered using intravenous injection. After the administration of cells (e.g., T cells) engineered to express one or more binders (e.g., antibodies, antigen binding fragments, antibody domains, CARs, or cell engagers) having the ability to bind to an ENPP1 polypeptide, the number of cancer cells (e.g., cancer cells expressing an ENPP1 polypeptide) within the human is reduced. After the administration of the cells (e.g., T cells) engineered to express one or more binders (e.g., antibodies, antigen binding fragments, antibody domains, CARs, or cell engagers) having the ability to bind to an ENPP1 polypeptide, the size of one or more tumors (e.g., tumors expressing an ENPP1 polypeptide) within the human is reduced.

### Example 22: Generation of T Cells Expressing One or More Binders Having the Ability to Bind to an ENPP1 Polypeptide

T cells are obtained from a human identified as having a sarcoma. Nucleic acid designed to express one or more binders (e.g., antibodies, antigen binding fragments, antibody domains, CARs, or cell engagers) having the ability to bind to an ENPP1 polypeptide provided herein is introduced into the T cell by transduction (e.g., viral transduction using a retroviral vector such as a lentiviral vector) or transfection such that the T cell expresses the binder(s) having the ability to bind to an ENPP1 polypeptide. The T cells engineered to express one or more binders (e.g., antibodies, antigen binding fragments, antibody domains, CARs, or cell engagers) having the ability to bind to an ENPP1 polypeptide are administered back into the human using, for example, intravenous injection. After the administration of the cells (e.g., T cells) engineered to express a one or more binders (e.g., antibodies, antigen binding fragments, antibody domains, CARs, or cell engagers) having the ability to bind to an ENPP1 polypeptide, the number of cancer cells (e.g., cancer cells expressing an ENPP1 polypeptide) within the human is reduced. After the administration of the cells (e.g., T cells) engineered to express a one or more binders (e.g., antibodies, antigen binding fragments, antibody domains, CARs, or cell engagers) having the ability to bind to an ENPP1 polypeptide, the size of one or more tumors (e.g., tumors expressing an ENPP1 polypeptide) within the human is reduced.

### Example 23: Exemplary Embodiments

Embodiment 1. An Antibody Comprising:
(i) a heavy chain variable domain or region comprising the amino acid sequences set forth in SEQ ID NO:1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO: 9 with one, two, or three amino acid additions, deletions, or substitutions), SEQ ID NO: 10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO: 17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO: 25 with one, two, or three amino acid additions, deletions, or substitutions), SEQ ID NO: 26 (or SEQ ID NO:26 with one, two, or three amino acid additions, deletions, or substitutions), and SEQ ID NO:27 (or SEQ ID NO:27 with one, two, or three amino acid additions, deletions, or substitutions).

Embodiment 2. The antibody of embodiment 1, wherein said antibody comprises the ability to bind to SEQ ID NO: 150 or SEQ ID NO:151.

Embodiment 3. The antibody of any one of embodiments 1-2, wherein said antibody comprises said heavy chain variable domain or region of said (i).

Embodiment 4. The antibody of embodiment 3, wherein said heavy chain variable domain or region comprises an amino acid sequence having at least 90 percent identity to the amino acid sequence set forth in SEQ ID NO:8.

Embodiment 5. The antibody of any one of embodiments 1-2, wherein said antibody comprises said light chain variable domain or region of said (i).

Embodiment 6. The antibody of embodiment 5, wherein said light chain variable domain or region comprises an amino acid sequence having at least 90 percent identity to the amino acid sequence set forth in SEQ ID NO:16.

Embodiment 7. The antibody of any one of embodiments 1-2, wherein said antibody comprises said heavy chain variable domain or region of said (ii).

Embodiment 8. The antibody of embodiment 7, wherein said heavy chain variable domain or region comprises an amino acid sequence having at least 90 percent identity to the amino acid sequence set forth in SEQ ID NO:24.

Embodiment 9. The antibody of any one of embodiments 1-2, wherein said antibody comprises said light chain variable domain or region of said (ii).

Embodiment 10. The antibody of embodiment 9, wherein said light chain variable domain or region comprises an amino acid sequence having at least 90 percent identity to the amino acid sequence set forth in SEQ ID NO:32.

Embodiment 11. An antigen binding fragment comprising:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO: 9 with one, two, or three amino acid additions, deletions, or substitutions), SEQ ID NO: 10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO: 17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO: 19 with one, two, or three amino acid additions, deletions, or substitutions), and a light chain variable domain or region comprising the amino acid sequences set forth in SEQ ID NO:25 (or SEQ ID NO: 25 with one, two, or three amino acid additions, deletions, or substitutions), SEQ ID NO: 26 (or SEQ ID NO:26 with one, two, or three amino acid additions, deletions, or substitutions), and SEQ ID NO:27 (or SEQ ID NO:27 with one, two, or three amino acid additions, deletions, or substitutions).

Embodiment 12. The antigen binding fragment of embodiment 11, wherein said antigen binding fragment comprises the ability to bind to SEQ ID NO:150 or SEQ ID NO:151.

Embodiment 13. The antigen binding fragment of any one of embodiments 11-12, wherein said antigen binding fragment comprises said heavy chain variable domain or region of said (i).

Embodiment 14. The antigen binding fragment of embodiment 13, wherein said heavy chain variable domain or region comprises an amino acid sequence having at least 90 percent identity to the amino acid sequence set forth in SEQ ID NO:8.

Embodiment 15. The antigen binding fragment of any one of embodiments 11-12, wherein said antigen binding fragment comprises said light chain variable domain or region of said (i).

Embodiment 16. The antigen binding fragment of embodiment 15, wherein said light chain variable domain or region comprises an amino acid sequence having at least 90 percent identity to the amino acid sequence set forth in SEQ ID NO:16.

Embodiment 17. The antigen binding fragment of any one of embodiments 11-12, wherein said antigen binding fragment comprises said heavy chain variable domain or region of said (ii).

Embodiment 18. The antigen binding fragment of embodiment 17, wherein said heavy chain variable domain or region comprises an amino acid sequence having at least 90 percent identity to the amino acid sequence set forth in SEQ ID NO:24.

Embodiment 19. The antigen binding fragment of any one of embodiments 11-12, wherein said antigen binding fragment comprises said light chain variable domain or region of said (ii).

Embodiment 20. The antigen binding fragment of embodiment 19, wherein said light chain variable domain or region comprises an amino acid sequence having at least 90 percent identity to the amino acid sequence set forth in SEQ ID NO:32.

Embodiment 21. The antibody of any one of embodiments 1-10, wherein said antibody is a monoclonal antibody.

Embodiment 22. The antibody of any one of embodiments 1-10 and 21, wherein said antibody is an scFv antibody.

Embodiment 23. The antigen binding fragment of any one of embodiments 11-20, wherein said antigen binding fragment is monoclonal.

Embodiment 24. The antigen binding fragment of any one of embodiments 11-20 and 23, wherein said antigen binding fragment is an Fab.

Embodiment 25. A chimeric antigen receptor comprising an antigen binding domain, a hinge, a transmembrane domain, and one or more signaling domains, wherein said antigen binding domain comprises an antibody or an antigen-binding fragment of any one of embodiments 1-23.

Embodiment 26. The chimeric antigen receptor of embodiment 25, wherein said antigen binding domain comprises a scFv having the ability to bind to an ectonucleotide pyrophosphatase/phosphodiesterase family member 1 (ENPP1) polypeptide.

Embodiment 27. The chimeric antigen receptor of any one of embodiments 25-26, wherein said hinge comprises a hinge set forth in any one of SEQ ID NOs: 105-112.

Embodiment 28. The chimeric antigen receptor of any one of embodiments 25-27, wherein said transmembrane domain comprises a transmembrane domain set forth in any one of SEQ ID NOs: 113-120.

Embodiment 29. The chimeric antigen receptor of any one of embodiments 25-28, wherein said chimeric antigen receptor comprises one or more signaling domains set forth in any one of SEQ ID NOs: 121-129.

Embodiment 30. A cell comprising a chimeric antigen receptor of any one of embodiments 25-29.

Embodiment 31. The cell of embodiment 30, wherein said cell is a T cell, a stem cell, or an NK cell.

Embodiment 32. A cell engager comprising a first antigen binding domain, a linker, and a second antigen binding domain, wherein said first antigen binding domain comprises an antibody or an antigen-binding fragment of any one of embodiments 1-24.

Embodiment 33. The cell engager of embodiment 32, wherein said first antigen binding domain comprises a scFv having the ability to bind to an ENPP1 polypeptide.

Embodiment 34. The cell engager of embodiment 32, wherein said first antigen binding domain is an IgG having the ability to bind to an ENPP1 polypeptide.

Embodiment 35. The cell engager of any one of embodiments 32-34, wherein said linker comprises a linker set forth in any one of SEQ ID NOs: 76-102 or SEQ ID NOs: 105-112.

Embodiment 36. The cell engager of any one of embodiments 32-35, wherein said second antigen binding domain binds to a polypeptide expressed on the surface of T cells.

Embodiment 37. The cell engager of embodiment 36, wherein said polypeptide expressed on the surface of T cells is a CD3 polypeptide.

Embodiment 38. The cell engager of embodiment 36, wherein said second antigen binding domain is an antigen binding domain set forth in Example 19.

Embodiment 39. The cell engager of any one of embodiments 32-35, wherein said second antigen binding domain binds to a polypeptide expressed on the surface of NK cells.

Embodiment 40. The cell engager of embodiment 39, wherein said polypeptide expressed on the surface of NK cells is a CD16a, NKG2A, NKG2D, NKp30, NKp44, or NKp46 polypeptide.

Embodiment 41. The cell engager of embodiment 39, wherein said second antigen binding domain is an antigen binding domain set forth in Example 20.

Embodiment 42. The cell engager of any one of embodiments 32-41, wherein said cell engager comprises a third antigen binding domain.

Embodiment 43. The cell engager of embodiment 42, wherein said third antigen binding domain binds to a polypeptide expressed on the surface of NK cells.

Embodiment 44. The cell engager of embodiment 43, wherein said polypeptide expressed on the surface of NK cells is a CD16a, NKG2A, NKG2D, NKp30, NKp44, or NKp46 polypeptide.

Embodiment 45. The cell engager of embodiment 43, wherein said third antigen binding domain is an antigen binding domain set forth in Example 20.

Embodiment 46. A nucleic acid comprising a nucleic acid sequence encoding at least part of an antibody or an antigen-binding fragment of any one of embodiments 1-24.

Embodiment 47. The nucleic acid of embodiment 46, wherein said nucleic acid sequence encodes said heavy chain variable domain or region of any one of said (i)-(ii) of embodiment 1.

Embodiment 48. The nucleic acid of any one of embodiments 46-47, wherein said nucleic acid sequence encodes said light chain variable domain or region of any one of said (i)-(ii) of embodiment 1.

Embodiment 49. The nucleic acid of any one of embodiments 46-48, wherein said nucleic acid is a viral vector.

Embodiment 50. The nucleic acid of any one of embodiments 46-48, wherein said nucleic acid is a phagemid.

Embodiment 51. A nucleic acid comprising a nucleic acid sequence encoding a chimeric antigen receptor of any one of embodiments 25-29 or a cell engager of any one of embodiments 32-45.

Embodiment 52. The nucleic acid of embodiment 51, wherein said nucleic acid is a viral vector.

Embodiment 53. The nucleic acid of embodiment 51, wherein said nucleic acid is a phagemid.

Embodiment 54. A host cell comprising a nucleic acid of any one of embodiments 46-52.

Embodiment 55. A host cell that expresses a chimeric antigen receptor of any one of embodiments 25-29 or a cell engager of any one of embodiments 32-45.

Embodiment 56. The host cell of any one of embodiments 54-55, wherein said host cell is a T cell, stem cell, or NK cell.

Embodiment 57. An antibody-drug conjugate (ADC) comprising an antigen binding domain covalently linked to a drug, wherein said antigen binding domain comprises an antibody or an antigen binding fragment of any one of embodiments 1-24.

Embodiment 58. The ADC of embodiment 57, wherein said antigen binding domain comprises a scFv having the ability to bind to an ENPP1 polypeptide.

Embodiment 59. The ADC of embodiment 57, wherein said antigen binding domain is an IgG having the ability to bind to an ENPP1 polypeptide.

Embodiment 60. The ADC of any one of embodiments 57-59, wherein said drug is selected from the group consisting of auristatins, mertansine, or pyrrolobenzodiazepine (PBD) dimers.

Embodiment 61. A composition comprising an antibody or an antigen binding fragment of any one of embodiments 1-24.

Embodiment 62. The composition of embodiment 61, wherein said composition comprises said antibody of any one of embodiments 1-10, 21, and 22.

Embodiment 63. The composition of embodiment 61, wherein said composition comprises said antigen binding fragment of any one of embodiments 11-20, 23, and 24.

Embodiment 64. A composition comprising a cell engager of any one of embodiments 32-45.

Embodiment 65. A composition comprising a cell of any one of embodiments 30, 31, and 53-56.

Embodiment 66. A composition comprising an ADC of any one of embodiments 57-60.

Embodiment 67. The composition of any one of embodiments 61-66, wherein said composition comprises a checkpoint inhibitor.

Embodiment 68. The composition of embodiment 67, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

Embodiment 69. A method of treating a mammal having cancer, wherein said method comprises administering, to said mammal, a composition of any one of embodiments 61-68.

Embodiment 70. The method of embodiment 69, wherein said mammal is a human.

Embodiment 71. The method of any one of embodiments 69-70, wherein said cancer is an ENPP1⁺ cancer.

Embodiment 72. The method of embodiment 71, wherein said ENPP1⁺ cancer is selected from the group consisting of an ENPP1⁺ sarcoma, an ENPP1⁺ lung cancer, an ENPP1⁺ breast cancer, and an ENPP1⁺ ovarian cancer.

Embodiment 73. The method of any one of embodiments 69-72, wherein the number of cancer cells within said mammal is reduced following said administering step.

Embodiment 74. A method of treating a mammal having cancer, wherein said method comprises:
(a) administering, to said mammal, said composition of any one of embodiments 61-68, and
(b) administering, to said mammal, a composition comprising a checkpoint inhibitor.

Embodiment 75. The method of embodiment 74, wherein said mammal is a human.

Embodiment 76. The method of any one of embodiments 74-75, wherein said cancer is an ENPP1⁺ cancer.

Embodiment 77. The method of embodiment 76, wherein said ENPP1⁺ cancer is selected from the group consisting of an ENPP1⁺ sarcoma, an ENPP1⁺ lung cancer, an ENPP1⁺ breast cancer, and an ENPP1⁺ ovarian cancer.

Embodiment 78. The method of any one of embodiments 74-77, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

Embodiment 79. The method of any one of embodiments 74-78, wherein the number of cancer cells within said mammal is reduced following said administering steps (a) and (b).

Embodiment 80. A method for binding a binding molecule to an ENPP1 polypeptide, wherein said method comprises contacting said ENPP1 polypeptide with an antibody or an antigen binding fragment of any one of embodiments 1-24.

Embodiment 81. The method of embodiment 80, wherein said contacting is performed in vitro.

Embodiment 82. The method of embodiment 80, wherein said contacting is performed in vivo.

Embodiment 83. The method of embodiment 82, wherein said contacting is performed within a mammal by administering said antibody or said antigen binding fragment to said mammal.

Embodiment 84. The method of embodiment 83, wherein said mammal is a human.

Embodiment 85. A method for binding a binding molecule to an ENPP1 polypeptide, wherein said method comprises contacting said ENPP1 polypeptide with a chimeric antigen receptor of any one of embodiments 25-29, a cell engager of any one of embodiments 32-45, or an ADC of any one of embodiments 57-60.

Embodiment 86. The method of embodiment 85, wherein said contacting is performed in vitro.

Embodiment 87. The method of embodiment 85, wherein said contacting is performed in vivo.

Embodiment 88. The method of embodiment 87, wherein said contacting is performed within a mammal by administering said chimeric antigen receptor, said cell engager, or said ADC to said mammal.

Embodiment 89. The method of embodiment 88, wherein said mammal is a human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO: 1, SEQ ID NO:2, and SEQ ID NO:3, and a light chain variable domain or region comprising the amino acid sequences set forth in SEQ ID NO:9, SEQ ID NO: 10, and SEQ ID NO: 11; or
(ii) a heavy chain variable domain or region comprising the amino acid sequences set forth in SEQ ID NO: 17, SEQ ID NO: 18, and SEQ ID NO: 19, and a light chain variable domain or region comprising the amino acid sequences set forth in SEQ ID NO:25, SEQ ID NO:26, and SEQ ID NO: 27.

2. The antibody of claim 1, wherein said antibody comprises the ability to bind to SEQ ID NO:150 or SEQ ID NO:151.

3. The antibody of claim 1, wherein said antibody comprises said heavy chain variable domain or region of said (i) and said light chain variable domain or region of said (i).

4-6. (canceled)

7. The antibody of claim 1, wherein said antibody comprises said heavy chain variable domain or region of said (ii) and said light chain variable domain or region of said (ii).

8-10. (canceled)

11. An antigen binding fragment comprising:
(i) a heavy chain variable domain or region comprising the amino acid sequences set forth in SEQ ID NO: 1, SEQ ID NO:2, and SEQ ID NO:3, and a light chain variable domain or region comprising the amino acid sequences set forth in SEQ ID NO:9, SEQ ID NO: 10, and SEQ ID NO:11; or
(ii) a heavy chain variable domain or region comprising the amino acid sequences set forth in SEQ ID NO: 17, SEQ ID NO: 18, and SEQ ID NO: 19, and a light chain variable domain or region comprising the amino acid sequences set forth in SEQ ID NO:25, SEQ ID NO:26, and SEQ ID NO: 27.

12. The antigen binding fragment of claim 11, wherein said antigen binding fragment comprises the ability to bind to SEQ ID NO: 150 or SEQ ID NO:151.

13. The antigen binding fragment of claim 11, wherein said antigen binding fragment comprises said heavy chain variable domain or region of said (i) and said light chain variable domain or region of said (i).

14-16. (canceled)

17. The antigen binding fragment of claim 11, wherein said antigen binding fragment comprises said heavy chain variable domain or region of said (ii) and said light chain variable domain or region of said (ii).

18-24. (canceled)

25. A chimeric antigen receptor comprising an antigen binding domain, a hinge, a transmembrane domain, and one or more signaling domains, wherein said antigen binding domain comprises:
(i) a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO: 11; or
(ii) a heavy chain variable domain or region comprising the amino acid sequences set forth in SEQ ID NO:17, SEQ ID NO:18, and SEQ ID NO:19, and a light chain variable domain or region comprising the amino acid sequences set forth in SEQ ID NO:25, SEQ ID NO:26, and SEQ ID NO:27.

26-29. (canceled)

30. A cell comprising a chimeric antigen receptor comprising an antigen binding domain, a hinge, a transmembrane domain, and one or more signaling domains, wherein said antigen binding domain comprises:
(i) a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO: 10, and SEQ ID NO: 11; or
(ii) a heavy chain variable domain or region comprising the amino acid sequences set forth in SEQ ID NO:17, SEQ ID NO:18, and SEQ ID NO:19, and a light chain variable domain or region comprising the amino acid sequences set forth in SEQ ID NO:25, SEQ ID NO:26, and SEQ ID NO:27.

31. (canceled)

32. A cell engager comprising a first antigen binding domain, a linker, and a second antigen binding domain, wherein said first antigen binding domain comprises:
(i) a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO:10, and SEQ ID NO: 11; or
(ii) a heavy chain variable domain or region comprising the amino acid sequences set forth in SEQ ID NO:17, SEQ ID NO:18, and SEQ ID NO:19, and a light chain variable domain or region comprising the amino acid sequences set forth in SEQ ID NO:25, SEQ ID NO:26, and SEQ ID NO:27.

33-56. (canceled)

57. An antibody-drug conjugate (ADC) comprising an antigen binding domain covalently linked to a drug, wherein said antigen binding domain comprises:
(i) a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO: 10, and SEQ ID NO: 11; or
(ii) a heavy chain variable domain or region comprising the amino acid sequences set forth in SEQ ID NO:17, SEQ ID NO: 18, and SEQ ID NO:19, and a light chain variable domain or region comprising the amino acid sequences set forth in SEQ ID NO:25, SEQ ID NO:26, and SEQ ID NO:27.

58-60. (canceled)

61. A composition comprising:
(a) an antibody or an antigen binding fragment comprising:
(i) a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO: 10, and SEQ ID NO:11; or
(ii) a heavy chain variable domain or region comprising the amino acid sequences set forth in SEQ ID NO:17, SEQ ID NO: 18, and SEQ ID NO:19, and a light chain variable domain or region comprising the amino acid sequences set forth in SEQ ID NO: 25, SEQ ID NO:26, and SEQ ID NO:27;
(b) a cell engager comprising a first antigen binding domain, a linker, and a second antigen binding domain, wherein said first antigen binding domain comprises said (i) or said (ii);
(c) a cell comprising a chimeric antigen receptor comprising an antigen binding domain, a hinge, a transmembrane domain, and one or more signaling domains, wherein said antigen binding domain comprises said (i) or said (ii); or
(d) an antibody-drug conjugate (ADC) comprising an antigen binding domain covalently linked to a drug, wherein said antigen binding domain comprises said (i) or said (ii).

62-66. (canceled)

67. The composition of claim 61, wherein said composition comprises a checkpoint inhibitor.

68. The composition of claim 67, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

69. A method of treating a mammal having cancer, wherein said method comprises administering, to said mammal, a composition comprising:
(a) an antibody or an antigen binding fragment comprising:
(i) a heavy chain variable domain or region comprising the amino acid sequences set forth in SEQ ID NO:1, SEQ ID NO:2, and SEQ ID NO:3, and a light chain variable domain or region comprising the amino acid sequences set forth in SEQ ID NO:9, SEQ ID NO: 10, and SEQ ID NO:11; or
(ii) a heavy chain variable domain or region comprising the amino acid sequences set forth in SEQ ID NO:17, SEQ ID NO:18, and SEQ ID NO:19, and a light chain variable domain or region comprising the amino acid sequences set forth in SEQ ID NO: 25, SEQ ID NO:26, and SEQ ID NO:27;
(b) a cell engager comprising a first antigen binding domain, a linker, and a second antigen binding domain, wherein said first antigen binding domain comprises said (i) or said (ii);
(c) a cell comprising a chimeric antigen receptor comprising an antigen binding domain, a hinge, a transmembrane domain, and one or more signaling domains, wherein said antigen binding domain comprises said (i) or said (ii); or
(d) an antibody-drug conjugate (ADC) comprising an antigen binding domain covalently linked to a drug, wherein said antigen binding domain comprises said (i) or said (ii).

70. The method of claim 69, wherein said mammal is a human.

71. The method of claim 69, wherein said cancer is an ENPP1⁺ cancer.

72. The method of claim 71, wherein said ENPP1⁺ cancer is selected from the group consisting of an ENPP1⁺ sarcoma, an ENPP1⁺ lung cancer, an ENPP1⁺ breast cancer, and an ENPP1⁺ ovarian cancer.

73. The method of claim 69, wherein the number of cancer cells within said mammal is reduced following said administering step.

74-89. (canceled)


SEQUENCE LISTING (304 sequences)

SEQ ID NO: 1 - protein, 8 aa, synthetic construct
GFTFSDYY

SEQ ID NO: 2 - protein, 8 aa, synthetic construct
ISSSGSTI

SEQ ID NO: 3 - protein, 19 aa, synthetic construct
AREVYYYDSSGYYTYYFDY

SEQ ID NO: 4 - protein, 25 aa, synthetic construct
QVQLVESGGGLVKPGGSLRLSCAAS

SEQ ID NO: 5 - protein, 17 aa, synthetic construct
MSWIRQAPGKGLEWVSY

SEQ ID NO: 6 - protein, 38 aa, synthetic construct
YYADSVKGRFTISRDNAKNSLYLQMNSLRAEDTAVYYC

SEQ ID NO: 7 - protein, 11 aa, synthetic construct
WGQGTLVTVSS

SEQ ID NO: 8 - protein, 126 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSS

SEQ ID NO: 9 - protein, 6 aa, synthetic construct
QSISSY

SEQ ID NO: 10 - protein, 7 aa, synthetic construct
AASSLQS

SEQ ID NO: 11 - protein, 9 aa, synthetic construct
QQYDTFPLT

SEQ ID NO: 12 - protein, 26 aa, synthetic construct [sequence omitted]

SEQ ID NO: 13 - protein, 17 aa, synthetic construct
LNWYQQKPGKAPKLLIY

SEQ ID NO: 14 - protein, 32 aa, synthetic construct
GVPSRFSGSGSGTDFTLTISSLQPEDAATYYC

SEQ ID NO: 15 - protein, 11 aa, synthetic construct
FGGGTKVEIKR

SEQ ID NO: 16 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKR

SEQ ID NO: 17 - protein, 8 aa, synthetic construct
GFTFSDYY

SEQ ID NO: 18 - protein, 8 aa, synthetic construct
ISSSGSTI

SEQ ID NO: 19 - protein, 19 aa, synthetic construct
AREVYYYDSSGYYTYYFDY

SEQ ID NO: 20 - protein, 25 aa, synthetic construct
QVQLVESGGGLVKPGGSLRLSCAAS

SEQ ID NO: 21 - protein, 17 aa, synthetic construct
MSWIRQAPGKGLEWVSY

SEQ ID NO: 22 - protein, 38 aa, synthetic construct; identical to SEQ ID NO: 6

SEQ ID NO: 23 - protein, 11 aa, synthetic construct
WGQGTLVTVSS

SEQ ID NO: 24 - protein, 126 aa, synthetic construct; identical to SEQ ID NO: 8

SEQ ID NO: 25 - protein, 6 aa, synthetic construct
QSISSY

SEQ ID NO: 26 - protein, 7 aa, synthetic construct
AASSLQS

SEQ ID NO: 27 - protein, 9 aa, synthetic construct
QQYYTSRIT

SEQ ID NO: 28 - protein, 26 aa, synthetic construct [sequence omitted]

SEQ ID NO: 29 - protein, 17 aa, synthetic construct
LNWYQQKPGKAPKLLIY

SEQ ID NO: 30 - protein, 32 aa, synthetic construct
GVPSRFSGSGSGTDFTLTISSLQPEDVAVYYC

SEQ ID NO: 31 - protein, 11 aa, synthetic construct
FGQGTKLEIKR

SEQ ID NO: 32 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCQQYYTSRITFGQGTKLEIKR

SEQ ID NO: 33 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 34 - protein, 107 aa, synthetic construct [sequence omitted]

SEQ ID NO: 35 - DNA, 378 bp, synthetic construct
  source 1..378: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctca

SEQ ID NO: 36 - DNA, 321 bp, synthetic construct
  source 1..321: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaa

SEQ ID NO: 37 - DNA, 378 bp, synthetic construct; identical to SEQ ID NO: 35
  source 1..378: mol_type = other DNA

SEQ ID NO: 38 - DNA, 321 bp, synthetic construct
  source 1..321: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcaacaatattacacttctcgtatcacgttcggccaagggaccaagctggaaatcaaa

SEQ ID NO: 39 - protein, 15 aa, synthetic construct
EPKSCDKTHTCPPCP

SEQ ID NO: 40 - DNA, 45 bp, synthetic construct
  source 1..45: mol_type = other DNA
gagcccaaatcttgtgacaaaactcacacatgcccaccgtgccca

SEQ ID NO: 41 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 651 bp, synthetic construct
  source 1..651: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43-44 - skipped (2 entries)

SEQ ID NO: 45 - protein, 259 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKRGGGGSGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSS

SEQ ID NO: 46 - DNA, 774 bp, synthetic construct
  source 1..774: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaaggagggggtggcagcggtggcgggggaagtggcggtggagggtcggggggaggcggttccgggggtggaggctcacaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctca

SEQ ID NO: 47 - protein, 249 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSS

SEQ ID NO: 48 - DNA, 744 bp, synthetic construct
  source 1..744: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaaggagggggtggcagcggtggcgggggaagtggcggtggagggtcgcaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctca

SEQ ID NO: 49 - protein, 259 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSSGGGGSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKR

SEQ ID NO: 50 - DNA, 774 bp, synthetic construct
  source 1..774: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctcaggagggggtggcagcggtggcgggggaagtggcggtggagggtcggggggaggcggttccgggggtggaggctcagatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaa

SEQ ID NO: 51 - protein, 249 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKR

SEQ ID NO: 52 - DNA, 744 bp, synthetic construct
  source 1..744: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctcaggagggggtggcagcggtggcgggggaagtggcggtggagggtcggatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaa

SEQ ID NO: 53-54 - skipped (2 entries)

SEQ ID NO: 55 - protein, 249 aa, synthetic construct; identical to SEQ ID NO: 47

SEQ ID NO: 56 - protein, 249 aa, synthetic construct; identical to SEQ ID NO: 51

SEQ ID NO: 57 - skipped

SEQ ID NO: 58 - protein, 25 aa, synthetic construct
EVQLVQSGGGLVQPGGSLRLSCSAS

SEQ ID NO: 59 - protein, 17 aa, synthetic construct
MHWVRQAPGKGLEYVSA

SEQ ID NO: 60 - protein, 38 aa, synthetic construct
YYADSVKGRFTISRDNSKNTLYLQMNSLRAEDTAVYYC

SEQ ID NO: 61 - protein, 11 aa, synthetic construct
WGQGTMVTVSS

SEQ ID NO: 62 - skipped

SEQ ID NO: 63 - protein, 26 aa, synthetic construct
EATLTQSPSSLSASVGDRVTITCRAS

SEQ ID NO: 64 - protein, 17 aa, synthetic construct
LNWYQQKPGRAPKLLIY

SEQ ID NO: 65 - protein, 36 aa, synthetic construct
SLQSGVPSRFSGSGSGTDFTLTISSLQPEDFATYYC

SEQ ID NO: 66 - skipped

SEQ ID NO: 67 - protein, 25 aa, synthetic construct
EVQLVQSGGGLVQPGGSLRLSCAAS

SEQ ID NO: 68 - protein, 17 aa, synthetic construct
MTWVRQAPGKGLEWVST

SEQ ID NO: 69 - protein, 38 aa, synthetic construct
YYADSVKGRFTISRDNDKNTLYLQMNSLRADDTAVYYC

SEQ ID NO: 70 - protein, 11 aa, synthetic construct
WGQGTLVTVSS

SEQ ID NO: 71 - skipped

SEQ ID NO: 72 - protein, 25 aa, synthetic construct
QSVVTQPPSASGSPGQSVTISCTGT

SEQ ID NO: 73 - protein, 17 aa, synthetic construct
VSWYQQHPGKAPKLLIY

SEQ ID NO: 74 - protein, 36 aa, synthetic construct
ERPSGVPDRFSGSKSDNKASLTIIGLQTEDEADYYC

SEQ ID NO: 75 - protein, 11 aa, synthetic construct
FGTGTKLTVLR

SEQ ID NO: 76 - protein, 15 aa, synthetic construct
GGGGSGGSGSGGGGS

SEQ ID NO: 77 - protein, 15 aa, synthetic construct
GGGGSGGSGSSGGGS

SEQ ID NO: 78 - protein, 15 aa, synthetic construct
GGGGSGGGGSGGGGS

SEQ ID NO: 79 - protein, 15 aa, synthetic construct
GGGGSGSGASSGGGS

SEQ ID NO: 80 - protein, 15 aa, synthetic construct
GGGGSGSGASGGGGS

SEQ ID NO: 81 - protein, 15 aa, synthetic construct
SGGGSGGGGSGGGGS

SEQ ID NO: 82 - protein, 18 aa, synthetic construct
GSTSGSGKPGSGEGSTKG

SEQ ID NO: 83 - protein, 19 aa, synthetic construct
PNGASQSSSASHTGSAPGS

SEQ ID NO: 84 - protein, 19 aa, synthetic construct
PNGASNSGSAPDTSSAPGS

SEQ ID NO: 85 - protein, 19 aa, synthetic construct
PNGASHSGSAPNTSSAPGS

SEQ ID NO: 86 - protein, 19 aa, synthetic construct
PNGASESGSASKTSSASGS

SEQ ID NO: 87 - protein, 19 aa, synthetic construct
PNGASNSGSAPKTGSASGS

SEQ ID NO: 88 - protein, 19 aa, synthetic construct
PNGASKSGSASQTSSAPGS

SEQ ID NO: 89 - protein, 19 aa, synthetic construct
PNGASHSSSASQTGSAPGS

SEQ ID NO: 90 - protein, 12 aa, synthetic construct
PNGASKRSAPGS

SEQ ID NO: 91 - protein, 19 aa, synthetic construct
PNGASHSGSAPHTSSASGS

SEQ ID NO: 92 - protein, 15 aa, synthetic construct
RGRGRGRGRSRGGGS

SEQ ID NO: 93 - protein, 15 aa, synthetic construct
SHGGSHGGGSGGGGS

SEQ ID NO: 94 - protein, 5 aa, synthetic construct
GQAGR

SEQ ID NO: 95 - protein, 8 aa, synthetic construct
GGGSGGGG

SEQ ID NO: 96 - protein, 5 aa, synthetic construct
SGGGG

SEQ ID NO: 97 - protein, 5 aa, synthetic construct
GGGGS

SEQ ID NO: 98 - protein, 30 aa, synthetic construct
GGGGSGGGGSGGGGSGGGGSGGGGSGGGGS

SEQ ID NO: 99 - protein, 16 aa, synthetic construct
GGGGGSGGGGSGGGGS

SEQ ID NO: 100 - protein, 26 aa, synthetic construct
GGGGGSGGGGSGGGGSGGGGSGGGGS

SEQ ID NO: 101 - protein, 25 aa, synthetic construct
GGGGSGGGGSGGGGSGGGGSGGGGS

SEQ ID NO: 102 - skipped

SEQ ID NO: 103 - protein, 22 aa, synthetic construct
MDMRVPAQLLGLLLLWLRGARC

SEQ ID NO: 104 - DNA, 66 bp, synthetic construct
  source 1..66: mol_type = other DNA
atggatatgagggtccccgcacaattgctcggtctccttctgctttggctccggggcgcgcggtgc

SEQ ID NO: 105 - protein, 15 aa, synthetic construct
EPKSCDKTHTCPPCP

SEQ ID NO: 106 - protein, 15 aa, synthetic construct
DKTHTCPPCPAPPVA

SEQ ID NO: 107 - protein, 12 aa, synthetic construct
ESKYGPPCPPCP

SEQ ID NO: 108 - protein, 12 aa, synthetic construct
ESKYGPPCPSCP

SEQ ID NO: 109 - protein, 49 aa, synthetic construct
KPTTTPAPRPPTPAPTIASQPLSLRPEACRPAAGGAVHTRGLDFACDIY

SEQ ID NO: 110 - protein, 39 aa, synthetic construct
IEVMYPPPYLDNERSNGTIIHVKGKHLCPSPLFPGPSKP

SEQ ID NO: 111 - protein, 47 aa, synthetic construct
TTTPAPRPPTPAPTIASQPLSLRPEACRPAAGGAVHTRGLDFACDIY

SEQ ID NO: 112 - protein, 45 aa, synthetic construct
TTTPAPRPPTPAPTIASQPLSLRPEACRPAAGGAVHTRGLDFACD

SEQ ID NO: 113 - protein, 21 aa, synthetic construct
LCYLLDGILFIYGVILTALFL

SEQ ID NO: 114 - protein, 22 aa, synthetic construct
MALIVLGGVAGLLLFIGLGIFF

SEQ ID NO: 115 - protein, 21 aa, synthetic construct
IYIWAPLAGTCGVLLLSLVIT

SEQ ID NO: 116 - protein, 24 aa, synthetic construct
IYIWAPLAGTCGVLLLSLVITLYC

SEQ ID NO: 117 - protein, 22 aa, synthetic construct
IWAPLAGTCGVLLLSLVITLYC

SEQ ID NO: 118 - protein, 19 aa, synthetic construct
IWAPLAGTCGVLLLSLVIT

SEQ ID NO: 119 - protein, 27 aa, synthetic construct
FWVLVVVGGVLACYSLLVTVAFIIFWV

SEQ ID NO: 120 - protein, 21 aa, synthetic construct
FWLPIGCAAFVVVCILGCILI

SEQ ID NO: 121 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 122 - protein, 42 aa, synthetic construct
KRGRKKLLYIFKQPFMRPVQTTQEEDGCSCRFPEEEEGGCEL

SEQ ID NO: 123 - protein, 37 aa, synthetic construct
KRGRKKLLYIFKQPFMRPVQTTQEEDGCSCRFPEEEE

SEQ ID NO: 124 - protein, 41 aa, synthetic construct
RSKRSRLLHSDYMNMTPRRPGPTRKHYQPYAPPRDFAAYRS

SEQ ID NO: 125 - protein, 42 aa, synthetic construct
ALYLLRRDQRLPPDAHKPPGGGSFRTPIQEEQADAHSTLAKI

SEQ ID NO: 126 - protein, 38 aa, synthetic construct
CWLTKKKYSSSVHDPNGEYMFMRAVNTAKKSRLTDVTL

SEQ ID NO: 127 - protein, 24 aa, synthetic construct
LCARPRRSPAQEDGKVYINMPGRG

SEQ ID NO: 128 - protein, 52 aa, synthetic construct
YFLGRLVPRGRGAAEAATRKQRITETESPYQELQGQRSDVYSDLNTQRPYYK

SEQ ID NO: 129 - protein, 48 aa, synthetic construct
QRRKYRSNKGESPVEPAEPCHYSCPREEEGSTIPIQEDYRKPEPACSP

SEQ ID NO: 130 - protein, 507 aa, synthetic construct
MLLLVTSLLLCELPHPAFLLIPDTSAQAADIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 131 - DNA, 1518 bp, synthetic construct
  source 1..1518: mol_type = other DNA
atgcttctgctcgtgacaagcctgctgctgtgcgagctgccccaccctgcctttctgctgatccctgacacctcggcccaggcggccgatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaaggagggggtggcagcggtggcgggggaagtggcggtggagggtcgcaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 132 - protein, 119 aa, synthetic construct [sequence omitted]

SEQ ID NO: 133 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 134 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 135 - protein, 108 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 136 - protein, 117 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 137 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 138 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 139 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 140 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 141 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 142 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 143 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 144 - protein, 107 aa, synthetic construct [sequence omitted]

SEQ ID NO: 145 - protein, 114 aa, synthetic construct
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 146 - protein, 112 aa, synthetic construct
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 147 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 148 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 149 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 150 - protein, 925 aa, synthetic construct
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 151 - skipped

SEQ ID NO: 152 - protein, 8 aa, synthetic construct
EPKSCDKT

SEQ ID NO: 153 - protein, 271 aa, synthetic construct
GGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 154 - DNA, 813 bp, synthetic construct
  source 1..813: mol_type = other DNA
ggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 155 - protein, 22 aa, synthetic construct
MDMRVLAQLLGLLLLCFPGARC

SEQ ID NO: 156 - protein, 20 aa, synthetic construct
MEAPAQLLFLLLLWLPDTTG

SEQ ID NO: 157 - protein, 20 aa, synthetic construct
MVLQTQVFISLLLWISGAYG

SEQ ID NO: 158 - protein, 19 aa, synthetic construct
MLPSQLIGFLLLWVPASRG

SEQ ID NO: 159 - protein, 22 aa, synthetic construct
MDMRVPAQLLGLLLLWLSGARC

SEQ ID NO: 160 - protein, 125 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 161 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 162-163 - skipped (2 entries)

SEQ ID NO: 164 - protein, 510 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKHHHHHH

SEQ ID NO: 165 - skipped

SEQ ID NO: 166 - protein, 8 aa, synthetic construct
GFEFSEYY

SEQ ID NO: 167 - protein, 8 aa, synthetic construct
GFTFSEYY

SEQ ID NO: 168 - protein, 8 aa, synthetic construct
GFEFSDYY

SEQ ID NO: 169 - protein, 8 aa, synthetic construct
GFGYSDYY

SEQ ID NO: 170 - protein, 8 aa, synthetic construct
GFTFSKEY

SEQ ID NO: 171 - protein, 8 aa, synthetic construct
GFTFAEYY

SEQ ID NO: 172 - protein, 8 aa, synthetic construct
GFTFTDYY

SEQ ID NO: 173 - protein, 8 aa, synthetic construct
GFTFSNYY

SEQ ID NO: 174 - protein, 8 aa, synthetic construct
GFTLSDYY

SEQ ID NO: 175 - protein, 8 aa, synthetic construct
YFTFSDYY

SEQ ID NO: 176 - protein, 8 aa, synthetic construct
ITSSGSTV

SEQ ID NO: 177 - protein, 8 aa, synthetic construct
ITSSGSTI

SEQ ID NO: 178 - protein, 8 aa, synthetic construct
INSSGSTI

SEQ ID NO: 179 - protein, 8 aa, synthetic construct
ISSSGSTV

SEQ ID NO: 180 - protein, 8 aa, synthetic construct
ISNSGSTI

SEQ ID NO: 181 - protein, 8 aa, synthetic construct
VSSSGSTI

SEQ ID NO: 182 - protein, 8 aa, synthetic construct
ISSSGTTI

SEQ ID NO: 183 - protein, 8 aa, synthetic construct
IASSGSTI

SEQ ID NO: 184 - protein, 8 aa, synthetic construct
ISSAGSTI

SEQ ID NO: 185 - protein, 8 aa, synthetic construct
ISSSGNTI

SEQ ID NO: 186 - protein, 19 aa, synthetic construct
AREVYYYDPTGYYGYYFNY

SEQ ID NO: 187 - protein, 19 aa, synthetic construct
ARDVYYYDPTGYYGYYFNY

SEQ ID NO: 188 - protein, 19 aa, synthetic construct
ARQIYYYGHDGYYEYIFDY

SEQ ID NO: 189 - protein, 19 aa, synthetic construct
ASEVYYYDSSGYYTYYFDY

SEQ ID NO: 190 - protein, 19 aa, synthetic construct
ARLVYFYDSSGYYTYYFDY

SEQ ID NO: 191 - protein, 19 aa, synthetic construct
AREVYYYDSSPYYQEYFDY

SEQ ID NO: 192 - protein, 19 aa, synthetic construct
ARQIYYYDSSGYYTYYFDY

SEQ ID NO: 193 - protein, 19 aa, synthetic construct
AREVYYYGHDGYYTYYFDY

SEQ ID NO: 194 - protein, 19 aa, synthetic construct
AREVYYYDSSGYYEYIFDY

SEQ ID NO: 195 - protein, 19 aa, synthetic construct
ARQVYYYGHDGYYTYYFDY

SEQ ID NO: 196 - protein, 6 aa, synthetic construct
QTVASY

SEQ ID NO: 197 - protein, 6 aa, synthetic construct
QSVSSY

SEQ ID NO: 198 - protein, 6 aa, synthetic construct
QSISTY

SEQ ID NO: 199 - protein, 6 aa, synthetic construct
QSISNY

SEQ ID NO: 200 - protein, 6 aa, synthetic construct
QSITSY

SEQ ID NO: 201 - protein, 6 aa, synthetic construct
QSISAY

SEQ ID NO: 202 - protein, 6 aa, synthetic construct
QSINSY

SEQ ID NO: 203 - protein, 6 aa, synthetic construct
QTISSY

SEQ ID NO: 204 - protein, 6 aa, synthetic construct
QSIASY

SEQ ID NO: 205 - protein, 6 aa, synthetic construct
QSVASY

SEQ ID NO: 206 - protein, 7 aa, synthetic construct
AATSLQS

SEQ ID NO: 207 - protein, 7 aa, synthetic construct
AASSMQS

SEQ ID NO: 208 - protein, 7 aa, synthetic construct
AASALQS

SEQ ID NO: 209 - protein, 7 aa, synthetic construct
AASGLQS

SEQ ID NO: 210 - protein, 7 aa, synthetic construct
AASTLQS

SEQ ID NO: 211 - protein, 7 aa, synthetic construct
AATSMQS

SEQ ID NO: 212 - protein, 7 aa, synthetic construct
AATALQS

SEQ ID NO: 213 - protein, 7 aa, synthetic construct
AATGLQS

SEQ ID NO: 214 - protein, 7 aa, synthetic construct
AATTLQS

SEQ ID NO: 215 - protein, 7 aa, synthetic construct
AASAMQS

SEQ ID NO: 216 - protein, 9 aa, synthetic construct
QQYGTFPLT

SEQ ID NO: 217 - protein, 9 aa, synthetic construct
QQYDSFPLT

SEQ ID NO: 218 - protein, 9 aa, synthetic construct
QQYNTFPLT

SEQ ID NO: 219 - protein, 9 aa, synthetic construct
QQYEPFPLT

SEQ ID NO: 220 - protein, 9 aa, synthetic construct
QKYDTFPLT

SEQ ID NO: 221 - protein, 9 aa, synthetic construct
QPYDTFPLT

SEQ ID NO: 222 - protein, 9 aa, synthetic construct
QEYDPFPLT

SEQ ID NO: 223 - protein, 9 aa, synthetic construct
QAYDTFPLT

SEQ ID NO: 224 - protein, 9 aa, synthetic construct
QQYDNFPLT

SEQ ID NO: 225 - protein, 9 aa, synthetic construct
QQYETFPLT

SEQ ID NO: 226 - protein, 8 aa, synthetic construct
GYTFSEYF

SEQ ID NO: 227 - protein, 8 aa, synthetic construct
GYTFSEYY

SEQ ID NO: 228 - protein, 8 aa, synthetic construct
GFRFSDYY

SEQ ID NO: 229 - protein, 8 aa, synthetic construct
GYTFNDYY

SEQ ID NO: 230 - protein, 8 aa, synthetic construct
GFTFSDHY

SEQ ID NO: 231 - protein, 8 aa, synthetic construct
GFSFSEYY

SEQ ID NO: 232 - protein, 8 aa, synthetic construct
GFTFNDYF

SEQ ID NO: 233 - protein, 8 aa, synthetic construct
GFTFPNYY

SEQ ID NO: 234 - protein, 8 aa, synthetic construct
GFTFTDFY

SEQ ID NO: 235 - protein, 8 aa, synthetic construct
GFTCSDYY

SEQ ID NO: 236 - protein, 8 aa, synthetic construct
ISASGTTI

SEQ ID NO: 237 - protein, 8 aa, synthetic construct
ISSSGTTI

SEQ ID NO: 238 - protein, 8 aa, synthetic construct
ISASGSTI

SEQ ID NO: 239 - protein, 8 aa, synthetic construct
ISSSGATI

SEQ ID NO: 240 - protein, 8 aa, synthetic construct
ISSTGSTI

SEQ ID NO: 241 - protein, 8 aa, synthetic construct
ISSSGSSI

SEQ ID NO: 242 - protein, 8 aa, synthetic construct
ISASGETI

SEQ ID NO: 243 - protein, 8 aa, synthetic construct
ISSSDSTI

SEQ ID NO: 244 - protein, 8 aa, synthetic construct
IPSSGSTI

SEQ ID NO: 245 - protein, 8 aa, synthetic construct
ISGSGSTI

SEQ ID NO: 246 - protein, 19 aa, synthetic construct
AREIYYYGHSGYYTYYFDY

SEQ ID NO: 247 - protein, 19 aa, synthetic construct
ARQIYYYGHSGYYTYYFDY

SEQ ID NO: 248 - protein, 19 aa, synthetic construct
ARQIYYYDHDGYYTYYFDY

SEQ ID NO: 249 - protein, 19 aa, synthetic construct
AREIYYYGHDGYYEYYFDY

SEQ ID NO: 250 - protein, 19 aa, synthetic construct
ARDVYYYDSSGYYGYYFNY

SEQ ID NO: 251 - protein, 19 aa, synthetic construct
AREVYYYDPTGYYTYYFDY

SEQ ID NO: 252 - protein, 19 aa, synthetic construct
ARDVYYYDPSGYYGYYFNY

SEQ ID NO: 253 - protein, 19 aa, synthetic construct
AREVYYYDSSGYYGYYFNY

SEQ ID NO: 254 - protein, 19 aa, synthetic construct
AREVYYYEGSGYYTYYFGY

SEQ ID NO: 255 - protein, 19 aa, synthetic construct
ARDVYYYDSSGYYTYYFNY

SEQ ID NO: 256 - protein, 6 aa, synthetic construct
QSVNAY

SEQ ID NO: 257 - protein, 6 aa, synthetic construct
QSVSTY

SEQ ID NO: 258 - protein, 6 aa, synthetic construct
QSVSNY

SEQ ID NO: 259 - protein, 6 aa, synthetic construct
QSVTNY

SEQ ID NO: 260 - protein, 6 aa, synthetic construct
QSVNSY

SEQ ID NO: 261 - protein, 6 aa, synthetic construct
QSVAAY

SEQ ID NO: 262 - protein, 6 aa, synthetic construct
QTINSY

SEQ ID NO: 263 - protein, 6 aa, synthetic construct
QTVSSY

SEQ ID NO: 264 - protein, 6 aa, synthetic construct
QTIASY

SEQ ID NO: 265 - protein, 6 aa, synthetic construct
QTVASY

SEQ ID NO: 266 - protein, 7 aa, synthetic construct
AASGMQS

SEQ ID NO: 267 - protein, 7 aa, synthetic construct
AASTMQS

SEQ ID NO: 268 - protein, 7 aa, synthetic construct
AATAMQS

SEQ ID NO: 269 - protein, 7 aa, synthetic construct
AATGMQS

SEQ ID NO: 270 - protein, 7 aa, synthetic construct
AATTMQS

SEQ ID NO: 271 - protein, 7 aa, synthetic construct
AATSLQS

SEQ ID NO: 272 - protein, 7 aa, synthetic construct
AASSMQS

SEQ ID NO: 273 - protein, 7 aa, synthetic construct
AATSMQS

SEQ ID NO: 274 - protein, 7 aa, synthetic construct
AASAMQS

SEQ ID NO: 275 - protein, 7 aa, synthetic construct
AASTMQS

SEQ ID NO: 276 - protein, 9 aa, synthetic construct
QQFYTTRIT

SEQ ID NO: 277 - protein, 9 aa, synthetic construct
QQYYTSGIT

SEQ ID NO: 278 - protein, 9 aa, synthetic construct
QQYYSSQIT

SEQ ID NO: 279 - protein, 9 aa, synthetic construct
QQYYTERIT

SEQ ID NO: 280 - protein, 9 aa, synthetic construct
QQYYTARIT

SEQ ID NO: 281 - protein, 9 aa, synthetic construct
QEYYTFRIT

SEQ ID NO: 282 - protein, 9 aa, synthetic construct
QQYYLNRIT

SEQ ID NO: 283 - protein, 9 aa, synthetic construct
QQYYTDHIT

SEQ ID NO: 284 - protein, 9 aa, synthetic construct
QQYYTGPIT

SEQ ID NO: 285 - protein, 9 aa, synthetic construct
QEYFTARIT

SEQ ID NO: 286 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 232 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 288 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCQQYYTSRITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 456 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - protein, 248 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSS

SEQ ID NO: 291 - protein, 248 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCQQYYTSRITFGQGTKLEIKGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAREVYYYDSSGYYTYYFDYWGQGTLVTVSS

SEQ ID NO: 292 - protein, 228 aa, synthetic construct
GQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 485 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQYDTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 294 - protein, 485 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCQQYYTSRITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 295 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 296 - DNA, 1371 bp, synthetic construct
  source 1..1371: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctccagcagcttgggcacccagacctacatctgcaacgtgaatcacaagcccagcaacaccaaggtcgacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaatag

SEQ ID NO: 297 - DNA, 696 bp, synthetic construct
  source 1..696: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctccagcagcttgggcacccagacctacatctgcaacgtgaatcacaagcccagcaacaccaaggtcgacaagaaagttgagcccaaatcttgtgacaaaact

SEQ ID NO: 298 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcaacaatattacacttctcgtatcacgttcggccaagggaccaagctggaaatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 299 - DNA, 744 bp, synthetic construct; identical to SEQ ID NO: 48
  source 1..744: mol_type = other DNA

SEQ ID NO: 300 - DNA, 744 bp, synthetic construct
  source 1..744: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcaacaatattacacttctcgtatcacgttcggccaagggaccaagctggaaatcaaaggagggggtggcagcggtggcgggggaagtggcggtggagggtcgcaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagagaggtatattactatgatagtagtggttattatacctactactttgactactggggccagggaaccctggtcactgtctcctca

SEQ ID NO: 301 - DNA, 687 bp, synthetic construct
  source 1..687: mol_type = other DNA
ggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 302 - DNA, 1371 bp, synthetic construct; identical to SEQ ID NO: 296
  source 1..1371: mol_type = other DNA

SEQ ID NO: 303 - DNA, 1458 bp, synthetic construct
  source 1..1458: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacaatatgatacttttccactcactttcggcggagggaccaaagtggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcctcagcagcaccctgacgctgagcaaagcagactacgaaaaacataaagtctacgcctgcgaagtcacccatcagggcctgagctcgcccgtcacaaagagcttcaacaggggagagtgtggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactaggtaa

SEQ ID NO: 304 - DNA, 1458 bp, synthetic construct
  source 1..1458: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcaacaatattacacttctcgtatcacgttcggccaagggaccaagctggaaatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcctcagcagcaccctgacgctgagcaaagcagactacgaaaaacataaagtctacgcctgcgaagtcacccatcagggcctgagctcgcccgtcacaaagagcttcaacaggggagagtgtggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactaggtaa